SEQUENCE LISTING (30 sequences)

SEQ ID NO: 1 - DNA, 28 bp, Artificial
  Novel Sequence
gacctcgaggttgcttaagactgaagca

SEQ ID NO: 2 - DNA, 28 bp, Artificial
  Novel Sequence
atttctagacatatgtagcttgtaccgt

SEQ ID NO: 3 - DNA, 50 bp, Artificial
  Novel Sequence
ctaggggcaccatgcaggctatcaacaatgaaagaaaagctaagaaagtc

SEQ ID NO: 4 - DNA, 50 bp, Artificial
  Novel Sequence
caaggactttcttagcttttctttcattgttgatagcctgcatggtgccc

SEQ ID NO: 5 - DNA, 31 bp, Artificial
  Novel Sequence
caaagaaagtactgggcatcgtcttcttcct

SEQ ID NO: 6 - DNA, 30 bp, Artificial
  Novel Sequence
tgctctagattccagataggtgaaaacttg

SEQ ID NO: 7 - DNA, 31 bp, Artificial
  Novel Sequence
ccgctcgagtactgcgccgacaagctttgat

SEQ ID NO: 8 - DNA, 38 bp, Artificial
  Novel Sequence
cgatgcccagcactttcgaagcttttctttcattgttg

SEQ ID NO: 9 - DNA, 36 bp, Artificial
  Novel Sequence
aaaagcttcgaaagtgctgggcatcgtcttcttcct

SEQ ID NO: 10 - DNA, 30 bp, Artificial; identical to SEQ ID NO: 6
  Novel Sequence

SEQ ID NO: 11 - DNA, 19 bp, Artificial
  Novel Sequence
cgtgtctctccttacttca

SEQ ID NO: 12 - DNA, 36 bp, Artificial
  Novel Sequence
tcggcgcagtactttgatagttagaaagtaggtgat

SEQ ID NO: 13 - DNA, 38 bp, Artificial
  Novel Sequence
ttctaactatcaaagtactgcgccgacaagctttgatg

SEQ ID NO: 14 - DNA, 43 bp, Artificial
  Novel Sequence
ttcagcagtcaacccactagtctatactctgttcaacaaaatt

SEQ ID NO: 15 - DNA, 28 bp, Artificial
  Novel Sequence
atttctagacatatgtagcttgtaccgt

SEQ ID NO: 16 - DNA, 19 bp, Artificial
  Novel Sequence
atcacctactttctaacta

SEQ ID NO: 17 - DNA, 33 bp, Artificial
  Novel Sequence
ccataatcgtcaggggaatgaaaaatgacacaa

SEQ ID NO: 18 - DNA, 33 bp, Artificial
  Novel Sequence
atttttcattcccctgacgattatggtgattac

SEQ ID NO: 19 - DNA, 33 bp, Artificial
  Novel Sequence
tgatgaagaaagggcaccacatgatcagaaaca

SEQ ID NO: 20 - DNA, 33 bp, Artificial
  Novel Sequence
gatcatgtggtgccctttcttcatcacaaacat

SEQ ID NO: 21 - DNA, 1416 bp, Homo sapiens
atggatattctttgtgaagaaaatacttctttgagctcaactacgaactccctaatgcaattaaatgatgacaacaggctctacagtaatgactttaactccggagaagctaacacttctgatgcatttaactggacagtcgactctgaaaatcgaaccaacctttcctgtgaagggtgcctctcaccgtcgtgtctctccttacttcatctccaggaaaaaaactggtctgctttactgacagccgtagtgattattctaactattgctggaaacatactcgtcatcatggcagtgtccctagagaaaaagctgcagaatgccaccaactatttcctgatgtcacttgccatagctgatatgctgctgggtttccttgtcatgcccgtgtccatgttaaccatcctgtatgggtaccggtggcctctgccgagcaagctttgtgcagtctggatttacctggacgtgctcttctccacggcctccatcatgcacctctgcgccatctcgctggaccgctacgtcgccatccagaatcccatccaccacagccgcttcaactccagaactaaggcatttctgaaaatcattgctgtttggaccatatcagtaggtatatccatgccaataccagtctttgggctacaggacgattcgaaggtctttaaggaggggagttgcttactcgccgatgataactttgtcctgatcggctcttttgtgtcatttttcattcccttaaccatcatggtgatcacctactttctaactatcaagtcactccagaaagaagctactttgtgtgtaagtgatcttggcacacgggccaaattagcttctttcagcttcctccctcagagttctttgtcttcagaaaagctcttccagcggtcgatccatagggagccagggtcctacacaggcaggaggactatgcagtccatcagcaatgagcaaaaggcatgcaaggtgctgggcatcgtcttcttcctgtttgtggtgatgtggtgccctttcttcatcacaaacatcatggccgtcatctgcaaagagtcctgcaatgaggatgtcattggggccctgctcaatgtgtttgtttggatcggttatctctcttcagcagtcaacccactagtctacacactgttcaacaagacctataggtcagccttttcacggtatattcagtgtcagtacaaggaaaacaaaaaaccattgcagttaattttagtgaacacaataccggctttggcctacaagtctagccaacttcaaatgggacaaaaaaagaattcaaagcaagatgccaagacaacagataatgactgctcaatggttgctctaggaaagcagtattctgaagaggcttctaaagacaatagcgacggagtgaatgaaaaggtgagctgtgtgtga

SEQ ID NO: 22 - protein, 471 aa, Homo sapiens
MDILCEENTSLSSTTNSLMQLNDDNRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQYSEEASKDNSDGVNEKVSCV

SEQ ID NO: 23 - DNA, 1377 bp, Homo sapiens
atggtgaacctgaggaatgcggtgcattcattccttgtgcacctaattggcctattggtttggcaatgtgatatttctgtgagcccagtagcagctatagtaactgacattttcaatacctccgatggtggacgcttcaaattcccagacggggtacaaaactggccagcactttcaatcgtcatcataataatcatgacaataggtggcaacatccttgtgatcatggcagtaagcatggaaaagaaactgcacaatgccaccaattacttcttaatgtccctagccattgctgatatgctagtgggactacttgtcatgcccctgtctctcctggcaatcctttatgattatgtctggccactacctagatatttgtgccccgtctggatttctttagatgttttattttcaacagcgtccatcatgcacctctgcgctatatcgctggatcggtatgtagcaatacgtaatcctattgagcatagccgtttcaattcgcggactaaggccatcatgaagattgctattgtttgggcaatttctataggtgtatcagttcctatccctgtgattggactgagggacgaagaaaaggtgttcgtgaacaacacgacgtgcgtgctcaacgacccaaatttcgttcttattgggtccttcgtagctttcttcataccgctgacgattatggtgattacgtattgcctgaccatctacgttctgcgccgacaagctttgatgttactgcacggccacaccgaggaaccgcctggactaagtctggatttcctgaagtgctgcaagaggaatacggccgaggaagagaactctgcaaaccctaaccaagaccagaacgcacgccgaagaaagaagaaggagagacgtcctaggggcaccatgcaggctatcaacaatgaaagaaaagcttcgaaagtccttgggattgttttctttgtgtttctgatcatgtggtgcccatttttcattaccaatattctgtctgttctttgtgagaagtcctgtaaccaaaagctcatggaaaagcttctgaatgtgtttgtttggattggctatgtttgttcaggaatcaatcctctggtgtatctctgtttcaacaaaatttaccgaagggcattctccaactatttgcgttgcaattataaggtagagaaaaagcctcctgtcaggcagattccaagagttgccgccactgctttgtctgggagggagcttaatgttaacatttatcggcataccaatgaaccggtgatcgagaaagccagtgacaatgagcccggtatagagatgcaagttgagaatttagagttaccagtaaatccctccagtgtggttagcgaaaggattagcagtgtgtga

SEQ ID NO: 24 - protein, 458 aa, Homo sapiens
MVNLRNAVHSFLVHLIGLLVWQCDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLHGHTEEPPGLSLDFLKCCKRNTAEEENSANPNQDQNARRRKKKERRPRGTMQAINNERKASKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 25 - DNA, 1377 bp, Artificial
  Novel Sequence
atggtgaacctgaggaatgcggtgcattcattccttgtgcacctaattggcctattggtttggcaatgtgatatttctgtgagcccagtagcagctatagtaactgacattttcaatacctccgatggtggacgcttcaaattcccagacggggtacaaaactggccagcactttcaatcgtcatcataataatcatgacaataggtggcaacatccttgtgatcatggcagtaagcatggaaaagaaactgcacaatgccaccaattacttcttaatgtccctagccattgctgatatgctagtgggactacttgtcatgcccctgtctctcctggcaatcctttatgattatgtctggccactacctagatatttgtgccccgtctggatttctttagatgttttattttcaacagcgtccatcatgcacctctgcgctatatcgctggatcggtatgtagcaatacgtaatcctattgagcatagccgtttcaattcgcggactaaggccatcatgaagattgctattgtttgggcaatttctataggtgtatcagttcctatccctgtgattggactgagggacgaagaaaaggtgttcgtgaacaacacgacgtgcgtgctcaacgacccaaatttcgttcttattgggtccttcgtagctttcttcataccgctgacgattatggtgattacgtattgcctgaccatctacgttctgcgccgacaagctttgatgttactgcacggccacaccgaggaaccgcctggactaagtctggatttcctgaagtgctgcaagaggaatacggccgaggaagagaactctgcaaaccctaaccaagaccagaacgcacgccgaagaaagaagaaggagagacgtcctaggggcaccatgcaggctatcaacaatgaaagaaaagctaagaaagtccttgggattgttttctttgtgtttctgatcatgtggtgcccatttttcattaccaatattctgtctgttctttgtgagaagtcctgtaaccaaaagctcatggaaaagcttctgaatgtgtttgtttggattggctatgtttgttcaggaatcaatcctctggtgtatactctgttcaacaaaatttaccgaagggcattctccaactatttgcgttgcaattataaggtagagaaaaagcctcctgtcaggcagattccaagagttgccgccactgctttgtctgggagggagcttaatgttaacatttatcggcataccaatgaaccggtgatcgagaaagccagtgacaatgagcccggtatagagatgcaagttgagaatttagagttaccagtaaatccctccagtgtggttagcgaaaggattagcagtgtgtga

SEQ ID NO: 26 - protein, 458 aa, Artificial
  Novel Sequence
MVNLRNAVHSFLVHLIGLLVWQCDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLHGHTEEPPGLSLDFLKCCKRNTAEEENSANPNQDQNARRRKKKERRPRGTMQAINNERKAKKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 27 - DNA, 1437 bp, Artificial
  Novel Sequence
atggatattctttgtgaagaaaatacttctttgagctcaactacgaactccctaatgcaattaaatgatgacaacaggctctacagtaatgactttaactccggagaagctaacacttctgatgcatttaactggacagtcgactctgaaaatcgaaccaacctttcctgtgaagggtgcctctcaccgtcgtgtctctccttacttcatctccaggaaaaaaactggtctgctttactgacagccgtagtgattattctaactattgctggaaacatactcgtcatcatggcagtgtccctagagaaaaagctgcagaatgccaccaactatttcctgatgtcacttgccatagctgatatgctgctgggtttccttgtcatgcccgtgtccatgttaaccatcctgtatgggtaccggtggcctctgccgagcaagctttgtgcagtctggatttacctggacgtgctcttctccacggcctccatcatgcacctctgcgccatctcgctggaccgctacgtcgccatccagaatcccatccaccacagccgcttcaactccagaactaaggcatttctgaaaatcattgctgtttggaccatatcagtaggtatatccatgccaataccagtctttgggctacaggacgattcgaaggtctttaaggaggggagttgcttactcgccgatgataactttgtcctgatcggctcttttgtgtcatttttcattcccttaaccatcatggtgatcacctactttctaactatcaaggttctgcgccgacaagctttgatgttactgcacggccacaccgaggaaccgcctggactaagtctggatttcctgaagtgctgcaagaggaatacggccgaggaagagaactctgcaaaccctaaccaagaccagaacgcacgccgaagaaagaagaaggagagacgtcctaggggcaccatgcaggctatcaacaatgaaagaaaagcttcgaaggtactgggcatcgtcttcttcctgtttgtggtgatgtggtgccctttcttcatcacaaacatcatggccgtcatctgcaaagagtcctgcaatgaggatgtcattggggccctgctcaatgtgtttgtttggatcggttatctctcttcagcagtcaacccactagtctatactctgttcaacaaaatttaccgaagggcattctccaactatttgcgttgcaattataaggtagagaaaaagcctcctgtcaggcagattccaagagttgccgccactgctttgtctgggagggagcttaatgttaacatttatcggcataccaatgaaccggtgatcgagaaagccagtgacaatgagcccggtatagagatgcaagttgagaatttagagttaccagtaaatccctccagtgtggttagcgaaaggattagcagtgtgtga

SEQ ID NO: 28 - protein, 478 aa, Artificial [sequence omitted]

SEQ ID NO: 29 - DNA, 1437 bp, Artificial
  Novel Sequence
atggatattctttgtgaagaaaatacttctttgagctcaactacgaactccctaatgcaattaaatgatgacaacaggctctacagtaatgactttaactccggagaagctaacacttctgatgcatttaactggacagtcgactctgaaaatcgaaccaacctttcctgtgaagggtgcctctcaccgtcgtgtctctccttacttcatctccaggaaaaaaactggtctgctttactgacagccgtagtgattattctaactattgctggaaacatactcgtcatcatggcagtgtccctagagaaaaagctgcagaatgccaccaactatttcctgatgtcacttgccatagctgatatgctgctgggtttccttgtcatgcccgtgtccatgttaaccatcctgtatgggtaccggtggcctctgccgagcaagctttgtgcagtctggatttacctggacgtgctcttctccacggcctccatcatgcacctctgcgccatctcgctggaccgctacgtcgccatccagaatcccatccaccacagccgcttcaactccagaactaaggcatttctgaaaatcattgctgtttggaccatatcagtaggtatatccatgccaataccagtctttgggctacaggacgattcgaaggtctttaaggaggggagttgcttactcgccgatgataactttgtcctgatcggctcttttgtgtcatttttcattcccctgacgattatggtgattacgtattgcctgaccatctacgttctgcgccgacaagctttgatgttactgcacggccacaccgaggaaccgcctggactaagtctggatttcctgaagtgctgcaagaggaatacggccgaggaagagaactctgcaaaccctaaccaagaccagaacgcacgccgaagaaagaagaaggagagacgtcctaggggcaccatgcaggctatcaacaatgaaagaaaagctaagaaagtccttgggattgttttctttgtgtttctgatcatgtggtgccctttcttcatcacaaacatcatggccgtcatctgcaaagagtcctgcaatgaggatgtcattggggccctgctcaatgtgtttgtttggatcggttatctctcttcagcagtcaacccactagtctatactctgttcaacaaaatttaccgaagggcattctccaactatttgcgttgcaattataaggtagagaaaaagcctcctgtcaggcagattccaagagttgccgccactgctttgtctgggagggagcttaatgttaacatttatcggcataccaatgaaccggtgatcgagaaagccagtgacaatgagcccggtatagagatgcaagttgagaatttagagttaccagtaaatccctccagtgtggttagcgaaaggattagcagtgtgtga

SEQ ID NO: 30 - protein, 478 aa, Artificial
  Novel Sequence
MDILCEENTSLSSTTNSLMQLNDDNRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYCLTIYVLRRQALMLLHGHTEEPPGLSLDFLKCCKRNTAEEENSANPNQDQNARRRKKKERRPRGTMQAINNERKAKKVLGIVFFVFLIMWCPFFITNIMAVICKESCNEDVIGALLNVFVWIGYLSSAVNPLVYTLFNKIYRRAFSNYLRCNYKVEKKPPVRQIPRVAATALSGRELNVNIYRHTNEPVIEKASDNEPGIEMQVENLELPVNPSSVVSERISSV

(19) United States
(12) Patent Application Publication
(10) Pub. No.: US 2008/0200530 A1
(43) Pub. Date: Aug. 21, 2008

(54) Diaryl and Arylheteroaryl Urea Derivatives as Modulators of 5-Ht2a Serotonin Receptor Useful for the Prophylaxis or Treatment of Progressive Multifocal Leukoencephalopathy

(76) Inventors: David J. Unett, San Diego, CA (US); Bradley Teegarden, San Diege, CA (US); Honnappa Jayakumar, San Diego, CA (US); Hongmei Li, Warren, NJ (US); Sonja Strah-Pleynet, San Diego, CA (US); Peter I. Dosa, San Diego, CA (US)

Correspondence Address: FISH & RICHARDSON P.C., P.O. BOX 1022, MINNEAPOLIS, MN, 55440-1022, US

(21) Appl. No.: 11/795,489

(22) PCT Filed: Jan. 17, 2006
(86) PCT No.: PCT/US06/01516

§ 371 Date: Jan. 11, 2008

Related U.S. Application Data
(60) Provisional application No. 60/645,532, filed on Jan. 19, 2005.

(51) Int. Cl. A61K 31/415; A61P 37/00; A61P 31/18; A61P 25/28

(52) U.S. Cl. 514/406

(57) ABSTRACT

The present invention relates to certain pyrazole derivatives of Formula (I) and pharmaceutical compositions thereof that modulate the activity of the 5-HT2A serotonin receptor. Compounds and pharmaceutical compositions thereof are directed to methods useful in the prophylaxis or treatment of progressive multifocal leukoencephalopathy.

### FIELD OF THE INVENTION

The present invention relates to certain diaryl and arylheteroaryl urea derivatives of Formula (I) and pharmaceutical compositions thereof that modulate the activity of the 5-HT_{2A} serotonin receptor. Compounds and pharmaceutical compositions thereof are directed to methods useful for the prophylaxis or treatment of progressive multifocal leukoencephalopathy.

### BACKGROUND OF THE INVENTION

G Protein Coupled Receptors

G Protein coupled receptors share a common structural motif. All these receptors have seven sequences of between 22 to 24 hydrophobic amino acids that form seven alpha helices, each of which spans the membrane. The transmembrane helices are joined by strands of amino acids having a larger loop between the fourth and fifth transmembrane helix on the extracellular side of the membrane. Another larger loop, composed primarily of hydrophilic amino acids, joins transmembrane helices five and six on the intracellular side of the membrane. The carboxy terminus of the receptor lies intracellularly with the amino terminus in the extracellular space. It is thought that the loop joining helices five and six, as well as, the carboxy terminus, interact with the G protein. Currently, Gq, Gs, Gi and Go are G proteins that have been identified. The general structure of G protein coupled receptors is shown in FIG. 1.

Under physiological conditions, G protein coupled receptors exist in the cell membrane in equilibrium between two different states or conformations: an “inactive” state and an “active” state. As shown schematically in FIG. 2, a receptor in an inactive state is unable to link to the intracellular transduction pathway to produce a biological response. Changing the receptor conformation to the active state allows linkage to the transduction pathway and produces a biological response.

A receptor may be stabilized in an active state by an endogenous ligand or an exogenous agonist ligand. Recent discoveries such as, including but not exclusively limited to, modifications to the amino acid sequence of the receptor provide means other than ligands to stabilize the active state conformation. These means effectively stabilize the receptor in an active state by simulating the effect of a ligand binding to the receptor. Stabilization by such ligand-independent means is termed “constitutive receptor activation.”

Serotonin Receptors

Receptors for serotonin (5-hydroxytryptamine, 5-HT) are an important class of G protein coupled receptors. Serotonin is thought to play a role in processes related to learning and memory, sleep, thermoregulation, mood, motor activity, pain, sexual and aggressive behaviors, appetite, neurodegenerative regulation, and biological rhythms. Serotonin receptors are divided into seven subfamilies, referred to as 5-HT1 through 5-HT7, inclusive. These subfamilies are further divided into subtypes. For example, the 5-HT2 subfamily is divided into three receptor subtypes: 5-HT_{2A}, 5-HT_{2B}, and 5-HT_{2C}. The human 5-HT_{2C} and 5-HT_{2A} receptors are thought to be the site of action of hallucinogenic drugs. Additionally, antagonists to the 5-HT_{2A} and 5-HT_{2C} receptors are believed to be useful in treating depression, anxiety, psychosis, and eating disorders.

Mutations of the endogenous forms of the rat 5-HT_{2A} and rat 5-HT_{2C} receptors have been reported to lead to constitutive activation of these receptors (5-HT_{2A}: Casey, C. et al. (1996) Society for Neuroscience Abstracts, 22:699.10, hereinafter “Casey”; 5-HT_{2C}: Herrick-Davis, K., and Teitler, M. (1996) Society for Neuroscience Abstracts, 22:699.18, hereinafter “Herrick-Davis 1”; and Herrick-Davis, K. et al. (1997) J. Neurochemistry 69(3): 1138, hereinafter “Herrick-Davis-2”). Casey describes mutations of the cysteine residue at position 322 of the rat 5-HT_{2A} receptor to lysine (C322K), glutamine (C322Q), and arginine (C322R) which reportedly led to constitutive activation. Herrick-Davis 1 and Herrick-Davis 2 describe mutations of the serine residue at position 312 of the rat 5-HT_{2C} receptor to phenylalanine (S312F) and lysine (S312K), which reportedly led to constitutive activation.

Progressive Multifocal Leukoencephalopathy

Progressive multifocal leukoencephalopathy (PML) is a lethal demyelinating disease caused by an opportunistic viral infection of oligodendrocytes in immunocompromised patients. The causative agent is JC virus, a ubiquitous papovavirus that infects the majority of the population before adulthood and establishes a latent infection in the kidney. In immunocompromised hosts, the virus can reactivate and productively infect oligodendrocytes. This previously rare condition, until 1984 reported primarily in persons with underlying lymphoproliferative disorders, is now more common because it occurs 4% of patients with AIDS. Patients usually present with relentlessly progressive focal neurologic defects, such as hemiparesis or visual field deficits, or with alterations in mental status. On brain MRI, one or more white matter lesions are present; they are hyperintense on T2-weighted images and hypointense on T1-weighted images. There is no mass effect, and contrast enhancement is rare. Diagnosis can be confirmed by brain biopsy, with demonstration of virus by in situ hybridization or immunocytochemistry. Polymerase chain reaction amplification of JC virus sequences from the CSF can confirm diagnosis without the need for biopsy [see, e.g., Antinori et al., Neurology (1997) 48:687-694; Berger and Major, Seminars in Neurology (1999) 19:193-200; and Portegies, et al., Eur. J. Neurol. (2004) 11:297-304]. Currently, there is no effective therapy. Survival after diagnosis is about 3 to 5 months in AIDS patients.

JC virus enters cells by receptor-mediated clathrin-dependent endocytosis. Binding of JC virus to human glial cells (e.g., oligodendrocytes) induces an intracellular signal that is critical for entry and infection by a ligand-inducible clathrin-dependent mechanism [Querbes et al., J Virology (2004) 78:250-256]. Recently, 5-HT_{2A} was shown to be the receptor on human glial cells mediating infectious entry of JC virus by clathrin-dependent endocytosis [Elphick et al., Science (2004) 306:1380-1383]. 5-HT_{2A} antagonists, including ketanserin and ritanserin, inhibited JC virus infection of human glial cells. Ketanserin and ritanserin have inverse agonist activity at 5-HT_{2A}.

5-HT_{2A} antagonists including inverse agonists have been contemplated to be useful in the treatment of PML [Elphick et al., Science (2004) 306:1380-1383]. Prophylactic treatment of HIV-infected patients with 5-HT_{2A} antagonists is envisioned to prevent the spread of JC virus to the central nervous system and the development of PML. Aggressive therapeutic treatment of patients with PML is envisioned to reduce viral spread within the central nervous system and prevent additional episodes of demyelination.

### SUMMARY OF THE INVENTION

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I):

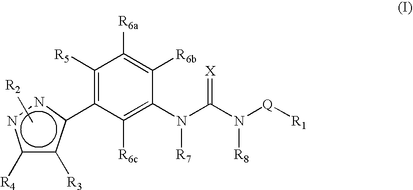

or a pharmaceutically acceptable salt, hydrate or solvate thereof;

wherein:

i) R₁ is aryl or heteroaryl each optionally substituted with R₉, R₁₀, R₁₁, R₁₂, R₁₃, R₁₄, and R₁₅ each selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F, Cl, or Br; and wherein said C₂₋₆ alkenyl, C₁₋₆ alkyl, C₂₋₆ alkynyl, C₁₋₆ alkylamino, C₁₋₆ alkylimino, C₂₋₈ dialkylamino, heterocyclic, and phenyl are each optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;

ii) R₂ is selected from the group consisting of H, C₁₋₆ alkyl, C₂₋₆ alkenyl, C₂₋₆ alkynyl and C₃₋₇ cycloalkyl;

iii) R₃ is selected from the group consisting of H, C₂₋₆ alkenyl, C₁₋₆ alkyl, C₁₋₆ alkylcarboxamide, C₂₋₆ alkynyl, C₁₋₆ alkylsulfonamide, carbo-C₁₋₆-alkoxy, carboxamide, carboxy, cyano, C₃₋₇ cycloalkyl, C₂₋₈ dialkylcarboxamide, halogen, heteroaryl and phenyl; and wherein each of said C₂₋₆ alkenyl, C₁₋₆ alkyl, C₂₋₆ alkynyl, C₁₋₆ alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and sulfonamide;

iv) R₄ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

v) R₅ is selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide, wherein said C₁₋₆ alkoxy group can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, amino, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and phenyl, and wherein said amino and phenyl are each optionally substituted with 1 to 5 further substituents selected from the group consisting of halogen and carbo-C₁₋₆-alkoxy;

vi) R₆ₐ, R_{6b}, and R_{6c} are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

vii) R₇ and R₈ are independently H or C₁₋₈ alkyl;

viii) X is O or S; and

ix) Q is C₁₋₃ alkylene optionally substituted with 1 to 4 substituents selected from the group consisting of C₁₋₃ alkyl, C₁₋₄ alkoxy, carboxy, cyano, C₁₋₃ haloalkyl, halogen and oxo; or Q is a bond.

One aspect of the present invention relates to a method of prophylaxis of progressive multifocal encephalopathy comprising administering to an individual in need thereof a therapeutically effective amount of a compound of the invention, wherein the compound is a diaryl or arylheteroaryl urea derivative according to Formula (I).

One aspect of the present invention relates to a method of treatment of progressive multifocal encephalopathy comprising administering to an individual in need thereof a therapeutically effective amount of a compound of the invention, wherein the compound is a diaryl or arylheteroaryl urea derivative according to Formula (I).

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the individual in need thereof has a lymphoproliferative disorder.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the individual in need thereof has carcinomatosis.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the individual in need thereof is immunocompromised.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the individual in need thereof is infected with HIV.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), wherein the individual in need thereof is infected with HIV, and wherein the HIV-infected individual has a CD4+ cell count of ≦200/mm³.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), wherein the individual in need thereof is infected with HIV, and wherein the HIV-infected individual has AIDS.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), wherein the individual in need thereof is infected with HIV, and wherein the HIV-infected individual has AIDS-related complex (ARC).

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the individual in need thereof is undergoing immunosuppressive therapy.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the individual in need thereof is undergoing immunosuppressive therapy after organ transplantation.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the compound is selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, Compound 65, Compound 66, Compound 67, Compound 68, Compound 69, Compound 70, Compound 71, Compound 72, Compound 73, Compound 74, Compound 75, Compound 76, Compound 77, Compound 78, Compound 79, Compound 80, Compound 81, Compound 82, Compound 83, Compound 84, Compound 85, Compound 86, Compound 87, Compound 88, Compound 89, Compound 90, Compound 91, Compound 92, Compound 93, Compound 94, Compound 95, Compound 96, Compound 97, Compound 98, Compound 99, Compound 100, Compound 101, Compound 102, Compound 103, Compound 104, Compound 105, Compound 106, Compound 107, Compound 108, Compound 109, Compound 110, Compound 111, Compound 112, Compound 113, Compound 114, Compound 115, Compound 116, Compound 117, Compound 118, Compound 119, Compound 120, Compound 121, Compound 122, Compound 123, Compound 124, Compound 125, Compound 126, Compound 127, Compound 128, Compound 129, Compound 130, Compound 131, Compound 132, Compound 133, Compound 134, Compound 135, Compound 136, Compound 137, Compound 138, Compound 139, Compound 140, Compound 141, Compound 142, Compound 143, Compound 144, Compound 145, Compound 146, Compound 147, Compound 148, Compound 149, Compound 150, Compound 151, Compound 152, Compound 153, Compound 154, Compound 155, Compound 156, Compound 157, Compound 158, Compound 159, Compound 160, Compound 161, Compound 162, Compound 163, Compound 164, Compound 165, Compound 166, Compound 167, Compound 168, Compound 169, Compound 170, Compound 171, Compound 172, Compound 173, Compound 174, Compound 175, Compound 176, Compound 177, Compound 178, Compound 179, Compound 180, Compound 181, Compound 182, Compound 183, Compound 184, Compound 185, Compound 186, Compound 187, Compound 188, Compound 189, Compound 190, Compound 191, Compound 192, Compound 193, Compound 194, Compound 195, and Compound 196.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the compound is a 5-HT_{2A} ligand.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the compound is a selective 5-HT_{2A} ligand.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the compound inhibits JC virus infection of human glial cells.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the compound is a 5-HT_{2A} inverse agonist.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the compound is a selective 5-HT_{2A} inverse agonist.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the compound crosses the blood-brain barrier.

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I), and wherein the individual in need thereof is a human.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I):

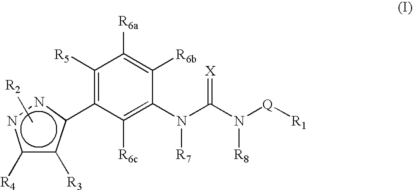

or a pharmaceutically acceptable salt, hydrate or solvate thereof;

wherein:

i) R₁ is aryl or heteroaryl each optionally substituted with R₉, R₁₀, R₁₁, R₁₂, R₁₃, R₁₄, and R₁₅ each selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F, Cl, or Br; and wherein said C₂₋₆ alkenyl, C₁₋₆ alkyl, C₂₋₆ alkynyl, C₁₋₆ alkylamino, C₁₋₆ alkylimino, C₂₋₈ dialkylamino, heterocyclic, and phenyl are each optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;

ii) R₂ is selected from the group consisting of H, C₁₋₆ alkyl, C₂₋₆ alkenyl, C₂₋₆ alkynyl and C₃₋₇ cycloalkyl;

iii) R₃ is selected from the group consisting of H, C₂₋₆ alkenyl, C₁₋₆ alkyl, C₁₋₆ alkylcarboxamide, C₂₋₆ alkynyl, C₁₋₆ alkylsulfonamide, carbo-C₁₋₆-alkoxy, carboxamide, carboxy, cyano, C₃₋₇ cycloalkyl, C₂₋₈ dialkylcarboxamide, halogen, heteroaryl and phenyl; and wherein each of said C₂₋₆ alkenyl, C₁₋₆ alkyl, C₂₋₆ alkynyl, C₁₋₆ alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and sulfonamide;

iv) R₄ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

v) R₅ is selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide, wherein said C₁₋₆ alkoxy group can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, amino, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and phenyl, and wherein said amino and phenyl are each optionally substituted with 1 to 5 further substituents selected from the group consisting of halogen and carbo-C₁₋₆-alkoxy;

vi) R₆ₐ, R_{6b}, and R_{6c} are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

vii) R₇ and R₈ are independently H or C₁₋₈ alkyl;

viii) X is O or S; and

ix) Q is C₁₋₃ alkylene optionally substituted with 1 to 4 substituents selected from the group consisting of C₁₋₃ alkyl, C₁₋₄ alkoxy, carboxy, cyano, C₁₋₃ haloalkyl, halogen and oxo; or Q is a bond.

One aspect of the present invention relates to a method of prophylaxis of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I).

One aspect of the present invention relates to a method of treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I).

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the individual in need thereof has a lymphoproliferative disorder.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the individual in need thereof has carcinomatosis.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the individual is immunocompromised.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the individual in need thereof is infected with HIV.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), wherein the individual in need thereof is infected with HIV, and wherein the HIV-infected individual has a CD4+ cell count of ≦200/mm³.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), wherein the individual in need thereof is infected with HIV, and wherein the HIV-infected individual has AIDS.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), wherein the individual in need thereof is infected with HIV, and wherein the HIV-infected individual has AIDS-related complex (ARC).

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the individual in need thereof is undergoing immunosuppressive therapy.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the individual in need thereof is undergoing immunosuppressive therapy after organ transplantation.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the compound is selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, Compound 65, Compound 66, Compound 67, Compound 68, Compound 69, Compound 70, Compound 71, Compound 72, Compound 73, Compound 74, Compound 75, Compound 76, Compound 77, Compound 78, Compound 79, Compound 80, Compound 81, Compound 82, Compound 83, Compound 84, Compound 85, Compound 86, Compound 87, Compound 88, Compound 89, Compound 90, Compound 91, Compound 92, Compound 93, Compound 94, Compound 95, Compound 96, Compound 97, Compound 98, Compound 99, Compound 100, Compound 101, Compound 102, Compound 103, Compound 104, Compound 105, Compound 106, Compound 107, Compound 108, Compound 109, Compound 110, Compound 111, Compound 112, Compound 113, Compound 114, Compound 115, Compound 116, Compound 117, Compound 118, Compound 119, Compound 120, Compound 121, Compound 122, Compound 123, Compound 124, Compound 125, Compound 126, Compound 127, Compound 128, Compound 129, Compound 130, Compound 131, Compound 132, Compound 133, Compound 134, Compound 135, Compound 136, Compound 137, Compound 138, Compound 139, Compound 140, Compound 141, Compound 142, Compound 143, Compound 144, Compound 145, Compound 146, Compound 147, Compound 148, Compound 149, Compound 150, Compound 151, Compound 152, Compound 153, Compound 154, Compound 155, Compound 156, Compound 157, Compound 158, Compound 159, Compound 160, Compound 161, Compound 162, Compound 163, Compound 164, Compound 165, Compound 166, Compound 167, Compound 168, Compound 169, Compound 170, Compound 171, Compound 172, Compound 173, Compound 174, Compound 175, Compound 176, Compound 177, Compound 178, Compound 179, Compound 180, Compound 181, Compound 182, Compound 183, Compound 184, Compound 185, Compound 186, Compound 187, Compound 188, Compound 189, Compound 190, Compound 191, Compound 192, Compound 193, Compound 194, Compound 195, and Compound 196.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the compound is a 5-HT_{2A} ligand.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the compound is a selective 5-HT_{2A} ligand.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the compound inhibits JC virus infection of human glial cells.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the compound is a 5-HT_{2A} inverse agonist.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the compound is a selective 5-HT_{2A} inverse agonist.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the compound crosses the blood-brain barrier.

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I), and wherein the individual in need thereof is a human.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I):

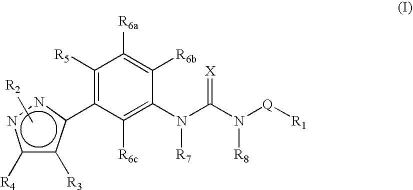

or a pharmaceutically acceptable salt, hydrate or solvate thereof;

wherein:

i) R₁ is aryl or heteroaryl each optionally substituted with R₉, R₁₀, R₁₁, R₁₂, R₁₃, R₁₄, and R₁₅ each selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F, Cl, or Br; and wherein said C₂₋₆ alkenyl, C₁₋₆ alkyl, C₂₋₆ alkynyl, C₁₋₆ alkylamino, C₁₋₆ alkylimino, C₂₋₈ dialkylamino, heterocyclic, and phenyl are each optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;

ii) R₂ is selected from the group consisting of H, C₁₋₆ alkyl, C₂₋₆ alkenyl, C₂₋₆ alkynyl and C₃₋₇ cycloalkyl;

iii) R₃ is selected from the group consisting of H, C₂₋₆ alkenyl, C₁₋₆ alkyl, C₁₋₆ alkylcarboxamide, C₂₋₆ alkynyl, C₁₋₆ alkylsulfonamide, carbo-C₁₋₆-alkoxy, carboxamide, carboxy, cyano, C₃₋₇ cycloalkyl, C₂₋₈ dialkylcarboxamide, halogen, heteroaryl and phenyl; and wherein each of said C₂₋₆ alkenyl, C₁₋₆ alkyl, C₂₋₆ alkynyl, C₁₋₆ alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and sulfonamide;

iv) R₄ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

v) R₅ is selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide, wherein said C₁₋₆ alkoxy group can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, amino, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and phenyl, and wherein said amino and phenyl are each optionally substituted with 1 to 5 further substituents selected from the group consisting of halogen and carbo-C₁₋₆-alkoxy;

vi) R₆ₐ, R_{6b}, and R_{6c} are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

vii) R₇ and R₈ are independently H or C₁₋₈ alkyl;

viii) X is O or S; and

ix) Q is C₁₋₃ alkylene optionally substituted with 1 to 4 substituents selected from the group consisting of C₁₋₃ alkyl, C₁₋₄ alkoxy, carboxy, cyano, C₁₋₃ haloalkyl, halogen and oxo; or Q is a bond.

One aspect of the present invention relates to a method of using a compound of the invention for the preparation of a medicament for the prophylaxis of progressive multifocal encephalopathy in an individual, wherein the compound is a diaryl or arylheteroaryl urea derivative according to Formula (I).

One aspect of the present invention relates to a method of using a compound of the invention for the preparation of a medicament for the treatment of progressive multifocal encephalopathy in an individual, wherein the compound is a diaryl or arylheteroaryl urea derivative according to Formula (I).

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the individual has a lymphoproliferative disorder.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the individual has carcinomatosis.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the individual is immunocompromised.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the individual is infected with HIV.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I) in an individual, wherein the individual is infected with HIV, and wherein the HIV-infected individual has a CD4+ cell count of ≦200/mm³.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), wherein the individual is infected with HIV, and wherein the HIV-infected individual has AIDS.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), wherein the individual is infected with HIV, and wherein the HIV-infected individual has AIDS-related complex.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the individual is undergoing immunosuppressive therapy.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the individual is undergoing immunosuppressive therapy after organ transplantation.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the compound is selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, Compound 65, Compound 66, Compound 67, Compound 68, Compound 69, Compound 70, Compound 71, Compound 72, Compound 73, Compound 74, Compound 75, Compound 76, Compound 77, Compound 78, Compound 79, Compound 80, Compound 81, Compound 82, Compound 83, Compound 84, Compound 85, Compound 86, Compound 87, Compound 88, Compound 89, Compound 90, Compound 91, Compound 92, Compound 93, Compound 94, Compound 95, Compound 96, Compound 97, Compound 98, Compound 99, Compound 100, Compound 101, Compound 102, Compound 103, Compound 104, Compound 105, Compound 106, Compound 107, Compound 108, Compound 109, Compound 110, Compound 111, Compound 112, Compound 113, Compound 114, Compound 115, Compound 116, Compound 117, Compound 118, Compound 119, Compound 120, Compound 121, Compound 122, Compound 123, Compound 124, Compound 125, Compound 126, Compound 127, Compound 128, Compound 129, Compound 130, Compound 131, Compound 132, Compound 133, Compound 134, Compound 135, Compound 136, Compound 137, Compound 138, Compound 139, Compound 140, Compound 141, Compound 142, Compound 143, Compound 144, Compound 145, Compound 146, Compound 147, Compound 148, Compound 149, Compound 150, Compound 151, Compound 152, Compound 153, Compound 154, Compound 155, Compound 156, Compound 157, Compound 158, Compound 159, Compound 160, Compound 161, Compound 162, Compound 163, Compound 164, Compound 165, Compound 166, Compound 167, Compound 168, Compound 169, Compound 170, Compound 171, Compound 172, Compound 173, Compound 174, Compound 175, Compound 176, Compound 177, Compound 178, Compound 179, Compound 180, Compound 181, Compound 182, Compound 183, Compound 184, Compound 185, Compound 186, Compound 187, Compound 188, Compound 189, Compound 190, Compound 191, Compound 192, Compound 193, Compound 194, Compound 195, and Compound 196.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the compound is a 5-HT_{2A} ligand.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the compound is a selective 5-HT_{2A} ligand.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the compound inhibits JC virus infection of human glial cells.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the compound is a 5-HT_{2A} inverse agonist.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the compound is a selective 5-HT_{2A} inverse agonist.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the compound crosses the blood-brain barrier.

One aspect of the present invention relates to a method of using a compound of the invention for the preparation of a medicament for the prophylaxis or treatment of progressive multifocal encephalopathy in an individual, wherein the compound is a diaryl or arylheteroaryl urea derivative according to Formula (I), and wherein the individual is a human.

These and other aspects of the invention disclosed herein will be set forth in greater detail as the patent disclosure proceeds.

This application claims the benefit of priority from the following provisional application, filed via U.S. Express mail with the United States Patent and Trademark Office on the indicated date: U.S. Provisional No. 60/645,532, filed Jan. 19, 2005. The disclosure of the foregoing application is herein incorporated by reference in its entirety.

### BRIEF DESCRIPTION OF THE DRAWINGS

In the following Figures, bold typeface indicates the location of the mutation in the non-endogenous, constitutively activated receptor relative to the corresponding endogenous receptor.

FIG. 1 shows a generalized structure of a G protein-coupled receptor with the numbers assigned to the transmembrane helices, the intracellular loops, and the extracellular loops.

FIG. 2 schematically shows the active and inactive states for a typical G protein-coupled receptor and the linkage of the active state to the second messenger transduction pathway.

FIG. 3a provides the nucleic acid sequence of the endogenous human 5-HT_{2A} receptor (SEQ.ID.NO: 21).

FIG. 3b provides the corresponding amino acid sequence of the endogenous human 5-HT_{2A} receptor (SEQ.ID.NO: 22).

FIG. 4a provides the nucleic acid sequence of the endogenous human 5-HT_{2C} receptor (SEQ.ID.NO: 23).

FIG. 4b provides the corresponding amino acid sequence of the endogenous human 5-HT_{2C} receptor (SEQ.ID.NO: 24).

FIG. 5a provides the nucleic acid sequence of a constitutively active form of the human 5-HT_{2C} receptor (“AP-1 cDNA”—SEQ.ID.NO: 25).

FIG. 5b provides the corresponding amino acid sequence of the AP-1 cDNA (“AP-1”—SEQ.ID.NO: 26).

FIG. 6a provides the nucleic acid sequence of a constitutively active form of the human 5-HT_{2A} receptor whereby the IC3 portion and the cytoplasmic-tail portion of the endogenous 5-HT_{2A} receptor have been replaced with the IC3 portion and the cytoplasmic-tail portion of the human 5-HT_{2C} receptor (“AP-3 cDNA”—SEQ.ID.NO: 27).

FIG. 6b provides the corresponding amino acid sequence of the AP-3 cDNA (“AP-3” SEQ.ID.NO: 28).

FIG. 6c provides a schematic representation of AP-3, where the dashed-lines represent the portion obtained from the human 5-HT_{2C} receptor.

FIG. 7a provides the nucleic acid sequence of a constitutively active form of the human 5-HT_{2A} receptor whereby (1) the region between the proline of TM5 and the proline of TM6 of the endogenous human 5-HT_{2A} receptor has been replaced with the corresponding region of the human 5-HT_{2C} receptor (including a S310K point mutation); and (2) the cytoplasmic-tail portion of the endogenous 5-HT_{2A} receptor has been replaced with the cytoplasmic-tail portion of the endogenous human 5-HT_{2C} receptor (“AP-4 cDNA”—SEQ.ID.NO: 29).

FIG. 7b provides the corresponding amino acid sequence of the AP-4 cDNA (“AP-4”—SEQ.ID.NO: 30).

FIG. 7c provides a schematic representation of the mutated 5-HT_{2A} receptor of FIG. 7b where the dashed-lines represent the portion obtained from the human 5-HT_{2C} receptor.

FIG. 8 is a representation of an exemplary expression vector, pCMV, used herein.

FIG. 9 is a diagram illustrating (1) enhanced (³⁵S)GTPγS binding to membranes prepared from COS cells expressing the endogenous human 5-HT_{2C} receptor in response to serotonin, and (2) inhibition by mianserin using wheatgerm agglutinin scintillation proximity beads. The concentration of (³⁵S)GTPγS was held constant at 0.3 nM, and the concentration of GDP was held at 1 μM. The concentration of the membrane protein was 12.5 μg.

FIG. 10 is a diagram showing serotonin stimulation of (³⁵S)GTPγS binding to membranes expressing AP-1 receptors in 293T cells and the inhibition by 30 μM mianserin on Wallac™ scintistrips.

FIG. 11 is a diagram showing the effects of protein concentration on (³⁵S)GTPγS binding in membranes prepared from 293T cells transfected with the endogenous human 5-HT_{2C} receptors and AP-1 receptors compared to cells transfected with the control vector (pCMV) alone in the absence (A) and presence (B) of 10 μM serotonin. The radiolabeled concentration of (³⁵S)GTPγS was held constant at 0.3 nM, and the GDP concentration was held constant at 1 μM. The assay was performed on 96-well format on Wallac™ scintistrips.

FIG. 12 provides bar-graph comparisons of inositol tris-phosphate (“IP3”) production between the endogenous human 5HT_{2A} receptor and AP-2, a mutated form of the receptor.

FIG. 13 provides bar-graph comparisons of inositol tris-phosphate (“IP3”) production between the endogenous human 5HT_{2A} receptor and AP-4, a mutated form of the receptor.

FIG. 14 provides bar graph comparisons of IP3 production between the endogenous human 5-HT_{2A} receptor and AP-3, a mutated form of the receptor.

FIG. 15 provides bar-graph comparisons of IP3 production between the endogenous human 5-HT_{2C} receptor and AP-1.

FIGS. 16A, 16B and 16C shows a grey-scale reproduction of representative autoradiograms demonstrating displacement of ¹²⁵I-LSD from brain sections by spiperone and a modulator of 5HT_{2A} identified as an early lead compound by the Inventors, referred to herein as S-1610 and having the following name: [3-(4-Bromo-2-methyl-2H-pyrazol-3-yl)-phenyl]-carbamic acid 4-methoxy-phenyl ester.

FIG. 17 shows the effect of Compound 1 on DOI-induced hypolocomotion in rats.

FIG. 18 shows the effect of Compound 26 on DOI-induced hypolocomotion in rats.

FIG. 19 shows the experimental design of 5HT_{2A} occupancy studies in monkeys.

FIG. 20 shows PET scan images of monkey brains 8 or 24 hours after treatment with Compound 1 compared to a baseline PET scan (transaxial view).

FIG. 21 shows PET scan images of monkey brains 8 or 24 hours after treatment with Compound 1 compared to a baseline PET scan (sagital view).

FIG. 22 shows tabulated data for percent occupancy of 5HT_{2A} receptors by Compound 1 in monkeys.

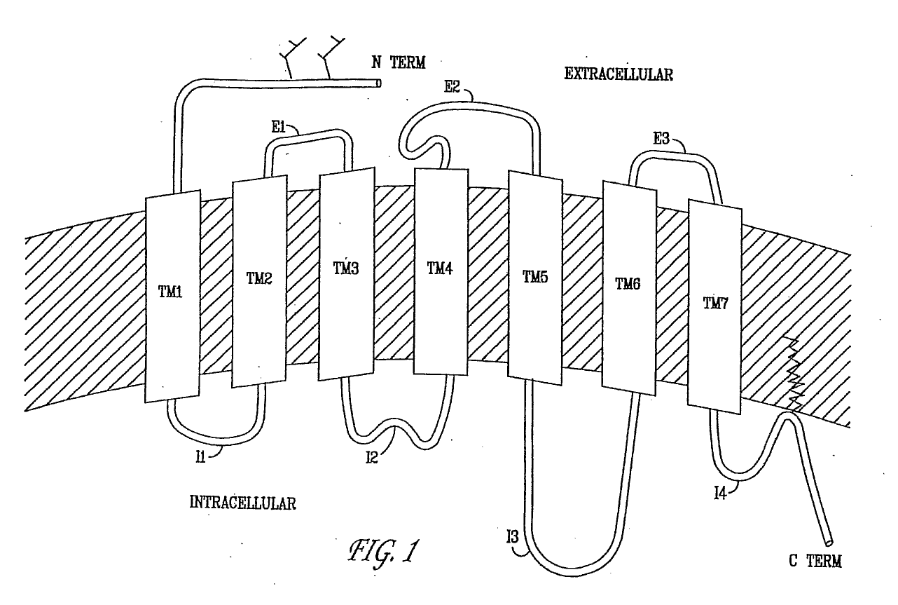

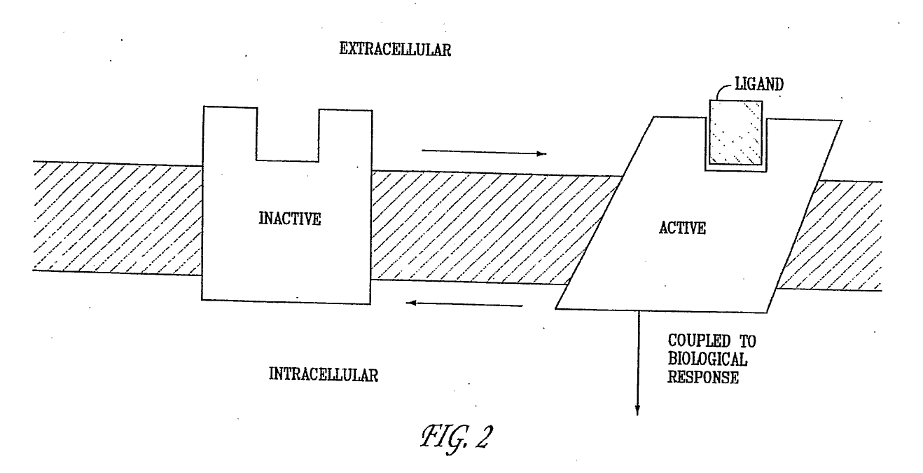

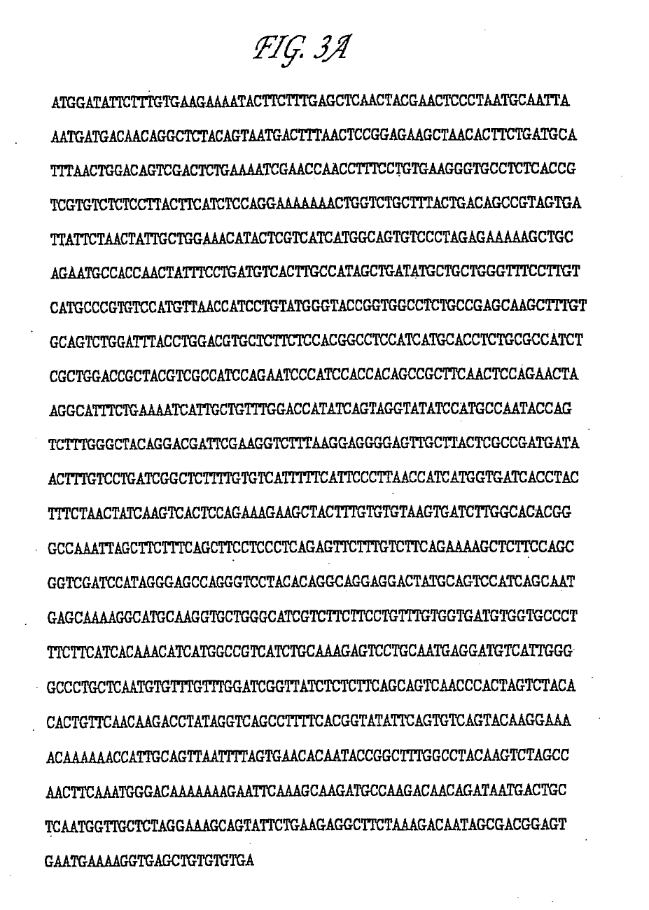

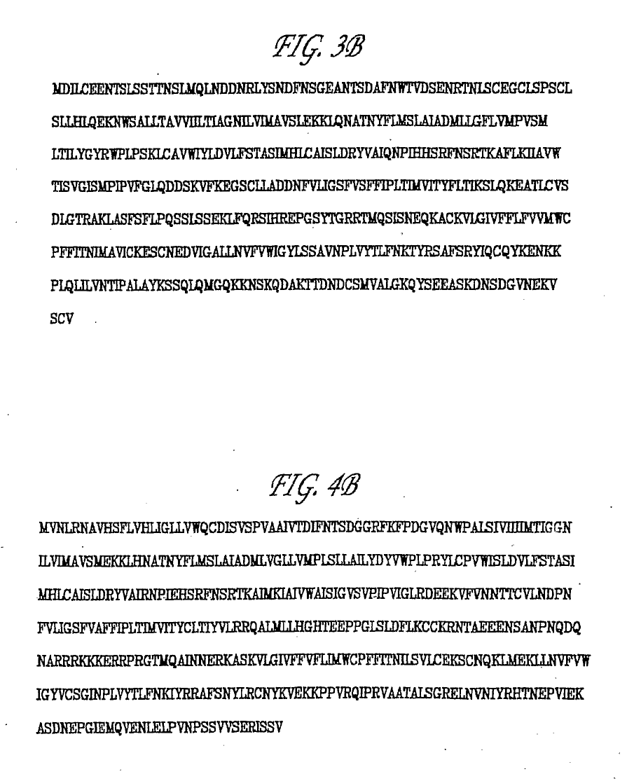

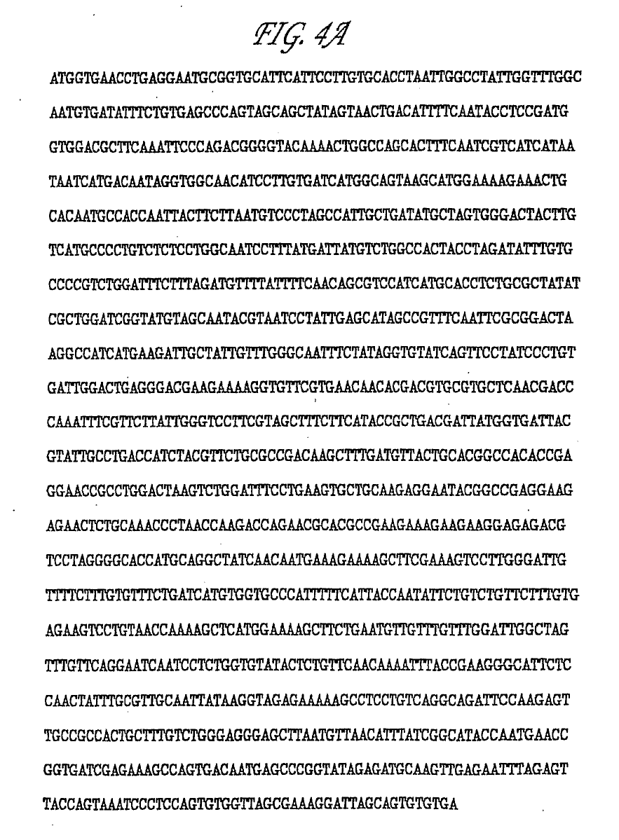

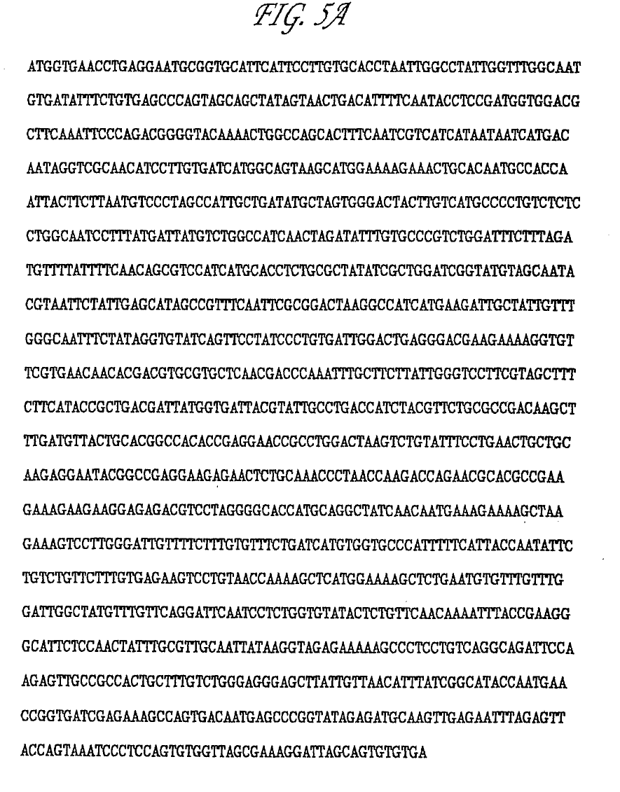

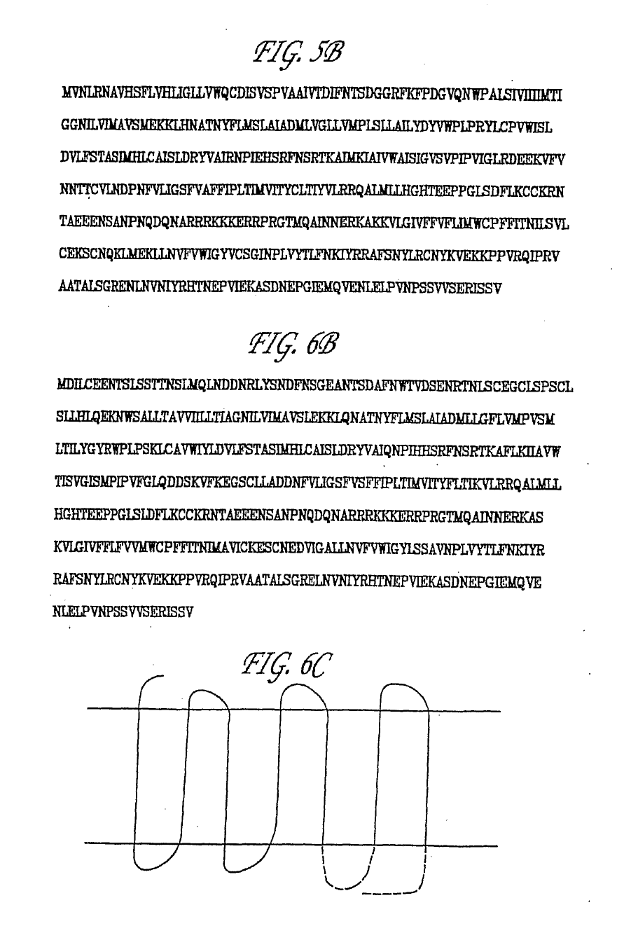

### DEFINITIONS

The scientific literature that has evolved around receptors has adopted a number of terms to refer to ligands having various effects on receptors. For clarity and consistency, the following definitions will be used throughout this patent document.

AGONISTS shall mean moieties that bind to and activate a receptor, such as the 5-HT_{2A} receptor, and initiate a physiological or pharmacological response characteristic of that receptor. For example, when moieties activate the intracellular response upon binding to the receptor, or enhance GTP binding to membranes. A SELECTIVE 5-HT_{2A} AGONIST is a 5-HT_{2A} agonist having a selectivity for 5-HT_{2A} over 5-HT_{2C}. In certain embodiments, a selective 5-HT_{2A} agonist is a 5-HT_{2A} agonist having a selectivity for 5-HT_{2A} over 5-HT_{2C} of at least about 10-fold. In certain embodiments, a selective 5-HT_{2A} agonist is a 5-HT_{2A} agonist having a selectivity for 5-HT_{2A} over 5-HT_{2C} of at least about 100-fold.

AMINO ACID ABBREVIATIONS used herein are set out in TABLE 1:

TABLE 1

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

The term ANTAGONISTS is intended to mean moieties that competitively bind to a receptor at the same site as agonists (for example, the endogenous ligand), but which do not activate the intracellular response initiated by the active form of the receptor, and can thereby inhibit the intracellular responses by agonists or partial agonists. Antagonists do not diminish the baseline intracellular response in the absence of an agonist or partial agonist. A SELECTIVE 5-HT_{2A} ANTAGONIST is a 5-HT_{2A} antagonist having a selectivity for 5-HT_{2A} over 5-HT_{2C}. In certain embodiments, a selective 5-HT_{2A} antagonist is a 5-HT_{2A} antagonist having a selectivity for 5-HT_{2A} over 5-HT_{2C} of at least about 10-fold. In certain embodiments, a selective 5-HT_{2A} antagonist is a 5-HT_{2A} antagonist having a selectivity for 5-HT_{2A} over 5-HT_{2C} of at least about 100-fold.

CHEMICAL GROUP, MOIETY OR RADICAL:

The term “C₁₋₆ acyl” denotes a C₁₋₆ alkyl radical attached to a carbonyl wherein the definition of alkyl has the same definition as described herein; some examples include but not limited to, acetyl, propionyl, n-butanoyl, iso-butanoyl, sec-butanoyl, t-butanoyl (i.e., pivaloyl), pentanoyl and the like.

The term “C₁₋₆ acyloxy” denotes an acyl radical attached to an oxygen atom wherein acyl has the same definition has described herein; some examples include but not limited to acetyloxy, propionyloxy, butanoyloxy, iso-butanoyloxy, sec-butanoyloxy, t-butanoyloxy and the like.

The term “C₂₋₆ alkenyl” denotes a radical containing 2 to 6 carbons wherein at least one carbon-carbon double bond is present, some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixtures of E and Z. Examples of an alkenyl include vinyl, alkyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl and the like.

The term “C₁₋₆ alkoxy” as used herein denotes a radical alkyl, as defined herein, attached directly to an oxygen atom. Examples include methoxy, ethoxy, n-propoxy, iso-propoxy, n-butoxy, t-butoxy, iso-butoxy, sec-butoxy and the like.

The term “C₁₋₈ alkyl” denotes a straight or branched carbon radical containing 1 to 8 carbons, some embodiments are 1 to 6 carbons, some embodiments are 1 to 4 carbons, some embodiments are 1 to 3 carbons, and some embodiments are 1 or 2 carbons. Examples of an alkyl include, but not limited to, methyl, ethyl, n-propyl, iso-propyl, n-butyl, sec-butyl, iso-butyl, t-butyl, pentyl, iso-pentyl, t-pentyl, neo-pentyl, 1-methylbutyl [i.e., —CH(CH₃)CH₂CH₂CH₃], 2-methylbutyl [i.e., —CH₂CH(CH₃)CH₂CH₃], n-hexyl and the like.

The term “C₁₋₆ alkylcarboxamido” or “C₁₋₆ alkylcarboxamide” denotes a single C₁₋₆ alkyl group attached to the nitrogen of an amide group, wherein alkyl has the same definition as found herein. The C₁₋₆ alkylcarboxamido may be represented by the following:

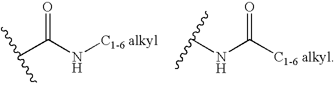

Examples include, but not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C₁₋₃ alkylene” refers to a C₁₋₃ divalent straight carbon group. In some embodiments C₁₋₃ alkylene refers to, for example, —CH₂—, —CH₂CH₂—, —CH₂CH₂CH₂—, and the like. In some embodiments, C₁₋₃ alkylene refers to —CH—, —CHCH₂—, —CHCH₂CH₂—, and the like wherein these examples relate generally to the variable or claim element “Q”.

The term “C₁₋₆ alkylimino” denotes a C₁₋₆ alkyl radical attached directly to the carbon of the —C(═NH)— group wherein the definition of alkyl has the same definition as described herein; some examples include but not limited to, 1-imino-ethyl [i.e., —C(═NH)CH₃], 1-imino-propyl [i.e., —C(═NH)CH₂CH₃], 1-imino-2-methyl-propyl [i.e., —C(=NH)CH(CH₃)₂], and the like.

The term “C₁₋₆ alkylsulfinyl” denotes a C₁₋₆ alkyl radical attached to a sulfoxide radical of the formula: —S(O)— wherein the alkyl radical has the same definition as described herein. Examples include, but not limited to, methylsulfinyl, ethylsulfinyl, n-propylsulfinyl, iso-propylsulfinyl, n-butylsulfinyl, sec-butylsulfinyl, iso-butylsulfinyl, t-butylsulfinyl, and the like.

The term “C₁₋₆ alkylsulfonamide” refers to the groups

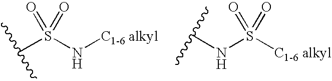

wherein C₁₋₆ alkyl has the same definition as described herein.

The term “C₁₋₆ alkylsulfonyl” denotes a C₁₋₆ alkyl radical attached to a sulfone radical of the formula: —S(O)₂— wherein the alkyl radical has the same definition as described herein. Examples include, but not limited to, methylsulfonyl, ethylsulfonyl, n-propylsulfonyl, iso-propylsulfonyl, n-butylsulfonyl, sec-butylsulfonyl, iso-butylsulfonyl, t-butylsulfonyl, and the like.

The term “C₁₋₆ alkylthio” denotes a C₁₋₆ alkyl radical attached to a sulfide of the formula: —S— wherein the alkyl radical has the same definition as described herein. Examples include, but not limited to, methylsulfanyl (i.e., CH₃S—), ethylsulfanyl, n-propylsulfanyl, iso-propylsulfanyl, n-butylsulfanyl, sec-butylsulfanyl, iso-butylsulfanyl, t-butylsulfanyl, and the like.

The term “C₁₋₆ alkylthiocarboxamide” denotes a thioamide of the following formulae:

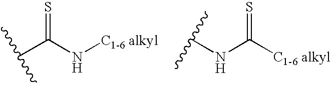

wherein C₁₋₄ alkyl has the same definition as described herein.

The term “C₁₋₆ alkylthioureyl” denotes the group of the formula: —NC(S)N— wherein one are both of the nitrogens are substituted with the same or different C₁₋₆ alkyl groups and alkyl has the same definition as described herein. Examples of an alkylthioureyl include, but not limited to, CH₃NHC(S)NH—, NH₂C(S)NCH₃—, (CH₃)₂N(S)NH—, (CH₃)₂N(S)NH—, (CH₃)₂N(S)NCH₃—, CH₃CH₂NHC(S)NH—, CH₃CH₂NHC(S)NCH₃—, and the like.

The term “C₁₋₆ alkylureyl” denotes the group of the formula: —NC(O)N— wherein one are both of the nitrogens are substituted with the same or different C₁₋₆ alkyl group wherein alkyl has the same definition as described herein. Examples of an alkylureyl include, but not limited to, CH₃NHC(O)NH—, NH₂C(O)NCH₃—, (CH₃)₂NC(O)NH—, (CH₃)₂NC(O)NH—, (CH₃)₂NC(O)NCH₃—, CH₃CH₂NHC(O)NH—, CH₃CH₂NHC(O)NCH₃—, and the like.

The term “C₂₋₆ alkynyl” denotes a radical containing 2 to 6 carbons and at least one carbon-carbon triple bond, some embodiments are 2 to 4 carbons, some embodiments are 2 to 3 carbons, and some embodiments have 2 carbons. Examples of an alkynyl include, but not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “amino” denotes the group —NH₂.

The term “C₁₋₆ alkylamino” denotes one alkyl radical attached to an amino radical wherein the alkyl radical has the same meaning as described herein. Some examples include, but not limited to, methylamino, ethylamino, n-propylamino, iso-propylamino, n-butylamino, sec-butylamino, iso-butylamino, t-butylamino, and the like. Some embodiments are “C₁₋₂ alkylamino.”

The term “aryl” denotes an aromatic ring radical containing 6 to 10 ring carbons. Examples include phenyl and naphthyl.

The term “arylalkyl” defines a C₁-C₄ alkylene, such as —CH₂—, —CH₂CH₂— and the like, which is further substituted with an aryl group. Examples of an “arylalkyl” include benzyl, phenethylene and the like.

The term “arylcarboxamido” denotes a single aryl group attached to the nitrogen of an amide group, wherein aryl has the same definition as found herein. The example is N-phenylcarboxamide.

The term “arylureyl” denotes the group —NC(O)N— where one of the nitrogens are substituted with an aryl.

The term “benzyl” denotes the group —CH₂C₆H₅.

The term “carbo-C₁₋₆-alkoxy” refers to a C₁₋₆ alkyl ester of a carboxylic acid, wherein the alkyl group is as defined herein. Examples include, but not limited to, carbomethoxy, carboethoxy, carbopropoxy, carboisopropoxy, carbobutoxy, carbo-sec-butoxy, carbo-iso-butoxy, carbo-t-butoxy, carbo-n-pentoxy, carbo-iso-pentoxy, carbo-t-pentoxy, carbo-neo-pentoxy, carbo-n-hexyloxy, and the like.

The term “carboxamide” refers to the group —CONH₂.

The term “carboxy” or “carboxyl” denotes the group —CO₂H; also referred to as a carboxylic acid group.

The term “cyano” denotes the group —CN.

The term “C₄₋₇ cycloalkenyl” denotes a non-aromatic ring radical containing 4 to 7 ring carbons and at least one double bond; some embodiments contain 4 to 6 carbons; some embodiments contain 4 to 5 carbons; some embodiments contain 4 carbons. Examples include cyclobutenyl, cyclopentenyl, cyclopentenyl, cyclohexenyl, and the like.

The term “C₃₋₇ cycloalkyl” denotes a saturated ring radical containing 3 to 7 carbons; some embodiments contain 3 to 6 carbons; some embodiments contain 3 to 5 carbons; some embodiments contain 5 to 7 carbons; some embodiments contain 3 to 4 carbons. Examples include cyclopropyl, cyclobutyl, cyclopentyl, cyclopenyl, cyclohexyl, cycloheptyl and the like.

The term “C₂₋₈ dialkylamino” denotes an amino substituted with two of the same or different C₁₋₄ alkyl radicals wherein alkyl radical has the same definition as described herein. Some examples include, but not limited to, dimethylamino, methylethylamino, diethylamino, methylpropylamino, methylisopropylamino, ethylpropylamino, ethylisopropylamino, dipropylamino, propylisopropylamino and the like. Some embodiments are “C₂₋₄ dialkylamino.”

The term “C₂₋₈ dialkylcarboxamido” or “C₂₋₈ dialkylcarboxamide” denotes two alkyl radicals, that are the same or different, attached to an amide group, wherein alkyl has the same definition as described herein. A C₂₋₈ dialkylcarboxamido may be represented by the following groups:

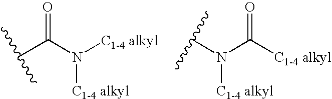

wherein C₁₋₄ has the same definition as described herein. Examples of a dialkylcarboxamide include, but not limited to, N,N-dimethylcarboxamide, N-methyl-N-ethylcarboxamide, N,N-diethylcarboxamide, N-methyl-N-isopropylcarboxamide, and the like.

The term “C₂₋₈ dialkylsulfonamide” refers to one of the following groups shown below:

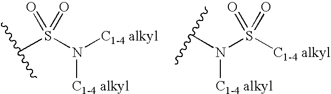

wherein C₁₋₄ has the same definition as described herein, for example but not limited to, methyl, ethyl, n-propyl, isopropyl, and the like.

The term “C₂₋₈ dialkylthiocarboxamido” or “C₂₋₈ dialkylthiocarbox-amide” denotes two alkyl radicals, that are the same or different, attached to a thioamide group, wherein alkyl has the same definition as described herein. A C₂₋₈ dialkylthiocarboxamido or C₂₋₈ dialkylthiocarboxamide may be represented by the following groups:

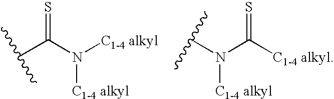

Examples of a dialkylthiocarboxamide include, but not limited to, N,N-dimethylthiocarboxamide, N-methyl-N-ethylthiocarboxamide and the like.

The term “ethynylene” refers to the carbon-carbon triple bond group as represented below:

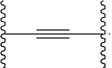

The term “formyl” refers to the group —CHO.

The term “C₁₋₆ haloalkoxy” denotes a haloalkyl, as defined herein, which is directly attached to an oxygen atom. Examples include, but not limited to, difluoromethoxy, trifluoromethoxy, 2,2,2-trifluoroethoxy, pentafluoroethoxy and the like.

The term “C₁₋₆ haloalkyl” denotes an C₁₋₆ alkyl group, defined herein, wherein the alkyl is substituted with one halogen up to fully substituted and a fully substituted C₁₋₆ haloalkyl can be represented by the formula CₙL₂ₙ₊₁ wherein L is a halogen and “n” is 1, 2, 3 or 4; when more than one halogen is present then they may be the same or different and selected from the group consisting of F, Cl, Br and I, preferably F. Examples of C₁₋₄ haloalkyl groups include, but not limited to, fluoromethyl, difluoromethyl, trifluoromethyl, chlorodifluoromethyl, 2,2,2-trifluoroethyl, pentafluoroethyl and the lice.

The term “C₁₋₆ haloalkylcarboxamide” denotes an alkylcarboxamide group, defined herein, wherein the alkyl is substituted with one halogen up to fully substituted represented by the formula CₙL₂ₙ₊₁ wherein L is a halogen and “n” is 1, 2, 3 or 4. When more than one halogen is present they may be the same or different and selected from the group consisting of F, Cl, Br and I, preferably F.

The term “C₁₋₆ haloalkylsulfinyl” denotes a haloalkyl radical attached to a sulfoxide group of the formula: —S(O)— wherein the haloalkyl radical has the same definition as described herein. Examples include, but not limited to, trifluoromethylsulfinyl, 2,2,2-trifluoroethylsulfinyl, 2,2-difluoroethylsulfinyl and the like.

The term “C₁₋₆ haloalkylsulfonyl” denotes a haloalkyl radical attached to a sulfone group of the formula: —S(O)₂— wherein haloalkyl has the same definition as described herein. Examples include, but not limited to, trifluoromethylsulfonyl, 2,2,2-trifluoroethylsulfonyl, 2,2-difluoroethylsulfonyl and the like.

The term “C₁₋₆ haloalkylthio” denotes a haloalkyl radical directly attached to a sulfur wherein the haloalkyl has the same meaning as described herein. Examples include, but not limited to, trifluoromethylthio (i.e., CF₃S—, also referred to as trifluoromethylsulfanyl), 1,1-difluoroethylthio, 2,2,2-trifluoroethylthio and the like.

The term “halogen” or “halo” denotes to a fluoro, chloro, bromo or iodo group. The term “heteroaryl” denotes an aromatic ring system that may be a single ring, two fused rings or three fused rings wherein at least one ring carbon is replaced with a heteroatom selected from, but not limited to, the group consisting of O, S and N wherein the N can be optionally substituted with H, C₁₋₄ acyl or C₁₋₄ alkyl. Examples of heteroaryl groups include, but not limited to, pyridyl, benzofuranyl, pyrazinyl, pyridazinyl, pyrimidinyl, triazinyl, quinoline, benzoxazole, benzothiazole, 1H-benzimidazole, isoquinoline, quinazoline, quinoxaline and the like. In some embodiments, the heteroaryl atom is O, S, NH, examples include, but not limited to, pyrrole, indole, and the like. Other examples include, but not limited to, those in TABLE 2, TABLE 3, and the like.

The term “heterocyclic” denotes a non-aromatic carbon ring (i.e., C₃₋₇ cycloalkyl or C₄₋₇ cycloalkenyl as defined herein) wherein one, two or three ring carbons are replaced by a heteroatom selected from, but not limited to, the group consisting of O, S, N, wherein the N can be optionally substituted with H, C₁₋₄ acyl or C₁₋₄ alkyl, and ring carbon atoms optionally substituted with oxo or a thiooxo thus forming a carbonyl or thiocarbonyl group. The heterocyclic group is a 3-, 4-, 5-, 6- or 7-membered containing ring. Examples of a heterocyclic group include but not limited to aziridin-1-yl, aziridin-2-yl, azetidin-1-yl, azetidin-2-yl, azetidin-3-yl, piperidin-1-yl, piperidin-4-yl, morpholin-4-yl, piperzin-1-yl, piperzin-4-yl, pyrrolidin-1-yl, pyrrolidin-3-yl, [1,3]-dioxolan-2-yl and the like.

The term “heterocycliccarboxamido” denotes a heterocyclic group, as defined herein, with a ring nitrogen where the ring nitrogen is bonded directly to the carbonyl forming an amide. Examples include, but not limited to,

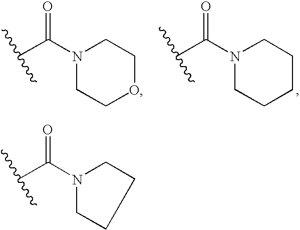

and the like.

The term “heterocyclicsulfonyl” denotes a heterocyclic group, as defined herein, with a ring nitrogen where the ring nitrogen is bonded directly to an —SO₂-group forming an sulfonamide. Examples include, but not limited to,

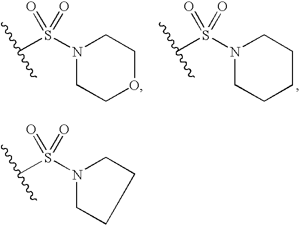

and the like.

The term “hydroxyl” refers to the group —OH.

The term “hydroxylamino” refers to the group —NHOH.

The term “nitro” refers to the group —NO₂.

The term “C₄₋₇ oxo-cycloalkyl” refers to a C₄₋₇ cycloalkyl, as defined herein, wherein one of the ring carbons is replaced with a carbonyl. Examples of C₄₋₇ oxo-cycloalkyl include, but are not limited to, 2-oxo-cyclobutyl, 3-oxo-cyclobutyl, 3-oxo-cyclopentyl, 4-oxo-cyclohexyl, and the like and represented by the following structures respectively:

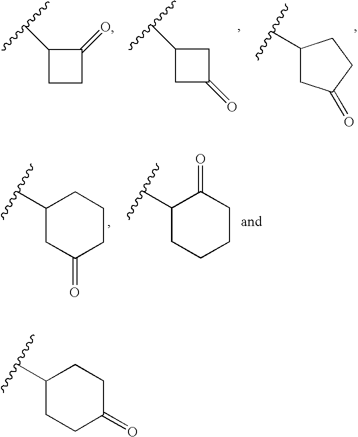

The term “perfluoroalkyl” denotes the group of the formula —CₙF₂ₙ₊₁; stated differently, a perfluoroalkyl is an alkyl as defined herein wherein the alkyl is fully substituted with fluorine atoms and is therefore considered a subset of haloalkyl. Examples of perfluoroalkyls include CF₃, CF₂CF₃, CF₂CF₂CF₃, CF(CF₃)₂, CF₂CF₂CF₂CF₃, CF₂CF(CF₃)₂, CF(CF₃)CF₂CF₃ and the like.

The term “phenoxy” refers to the group C₆H₅O—.

The term “phenyl” refers to the group C₆H₅—.

The term “sulfonic acid” refers to the group —SO₃H.

The term “thiol” denotes the group —SH.

CODON shall mean a grouping of three nucleotides (or equivalents to nucleotides) which generally comprise a nucleoside [adenosine (A), guanosine (G), cytidine (C), uridine (U) and thymidine (T)] coupled to a phosphate group and which, when translated, encodes an amino acid.

COMPOSITION shall mean a material comprising at least two compounds or two components; for example, and without limitation, a Pharmaceutical Composition is a Composition comprising a compound of the present invention and a pharmaceutically acceptable carrier.

COMPOUND EFFICACY shall mean a measurement of the ability of a compound to inhibit or stimulate receptor functionality, as opposed to receptor binding affinity.

CONSTITUTIVELY ACTIVATED RECEPTOR shall mean a receptor subject to constitutive receptor activation.

CONSTITUTIVE RECEPTOR ACTIVATION shall mean stabilization of a receptor in the active state by means other than binding of the receptor with its endogenous ligand or a chemical equivalent thereof.

CONTACT or CONTACTING shall mean bringing the indicated moieties together, whether in an in vitro system or an in vivo system. Thus, “contacting” a 5-HT_{2A} receptor with a compound of the invention includes the administration of a compound of the present invention to an individual, preferably a human, having a 5-HT_{2A} receptor, as well as, for example, introducing a compound of the invention into a sample containing a cellular or more purified preparation containing a 5-HT_{2A} receptor.

ENDOGENOUS shall mean a material that a mammal naturally produces. ENDOGENOUS in reference to, for example and not limitation, the term “receptor” shall mean that which is naturally produced by a mammal (for example, and not limitation, a human) or a virus.

In contrast, the term NON-ENDOGENOUS in this context shall mean that which is not naturally produced by a mammal (for example, and not limitation, a human) or a virus. For example, and not limitation, a receptor which is not constitutively active in its endogenous form, but when manipulated becomes constitutively active, is most preferably referred to herein as a “non-endogenous, constitutively activated receptor.” Both terms can be utilized to describe both “in vivo” and “in vitro” systems. For example, and not a limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

IN NEED OF PROPHYLAXIS OR TREATMENT as used herein refers to a judgment made by a caregiver (e.g. physician, nurse, nurse practitioner, etc. in the case of humans; veterinarian in the case of animals, including non-human mammals) that an individual or animal requires or will benefit from prophylaxis or treatment. This judgment is made based on a variety of factors that are in the realm of a caregiver's expertise, but that includes the knowledge that the individual or animal is ill, or will be ill, as the result of a disease, condition or disorder that is treatable by the compounds of the invention. In general, “in need of prophylaxis” refers to the judgment made by the caregiver that the individual will become ill. In this context, the compounds of the invention are used in a protective or preventive manner. However, “in need of treatment” refers to the judgment of the caregiver that the individual is already ill, therefore, the compounds of the present invention are used to alleviate, inhibit or ameliorate the disease, condition or disorder.

INDIVIDUAL as used herein refers to any animal, including mammals, preferably mice, rats, other rodents, rabbits, dogs, cats, swine, cattle, sheep, horses, or primates, and most preferably humans.

INHIBIT or INHIBITING, in relationship to the term “response” shall mean that a response is decreased or prevented in the presence of a compound as opposed to in the absence of the compound. Inhibit or inhibiting, in relationship to the term “JC virus infection of human glial cells” shall mean that JC virus infection of human glial cells is decreased or prevented in the presence of a compound as opposed to in the absence of the compound.

INVERSE AGONISTS shall mean moieties that bind the endogenous form of a receptor or to the constitutively activated form of the receptor, such as the 5-HT_{2A} receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist. A SELECTIVE 5-HT_{2A} INVERSE AGONIST is a 5-HT_{2A} inverse agonist having a selectivity for 5-HT_{2A} over 5-HT_{2C}. In certain embodiments, a selective 5-HT_{2A} inverse agonist is a 5-HT_{2A} inverse agonist having a selectivity for 5-HT_{2A} over 5-HT_{2C} of at least about 10-fold. In certain embodiments, a selective 5-HT_{2A} inverse agonist is a 5-HT_{2A} inverse agonist having a selectivity for 5-HT_{2A} over 5-HT_{2C} of at least about 100-fold.

LIGAND shall mean a moiety that specifically binds to an endogenous, naturally occurring receptor, such as the 5-HT_{2A} receptor, A SELECTIVE 5-HT_{2A} LIGAND is a 5-HT_{2A} ligand having a selectivity for 5-HT_{2A} over 5-HT_{2C}. In certain embodiments, a selective 5-HT_{2A} ligand is a 5-HT_{2A} ligand having a selectivity for 5-HT_{2A} over 5-HT_{2C} of at least about 10-fold. In certain embodiments, a selective 5-HT_{2A} ligand is a 5-HT_{2A} ligand having a selectivity for 5-HT_{2A} over 5-HT_{2C} of at least about 100-fold.

As used herein, the terms MODULATE or MODULATING shall mean to refer to an increase or decrease in the amount, quality, response or effect of a particular activity, function or molecule.

MODULATORS shall mean moieties that bind to and modulate a receptor, such as the 5-HT_{2A} receptor. By way of illustration and not limitation, agonists, antagonists, inverse agonists, and partial agonists are modulators.

PARTIAL AGONISTS shall mean moieties that bind to and activate a receptor, such as the 5-HT_{2A} receptor, and initiate a physiological or pharmacological response characteristic of that receptor, albeit to a lesser exent or degree than do full agonists. A SELECTIVE 5-HT_{2A} PARTIAL AGONIST is a 5-HT_{2A} partial agonist having a selectivity for 5-HT_{2A} over 5-HT_{2C}. In certain embodiments, a selective 5-HT_{2A} partial agonist is a 5-HT_{2A} partial agonist having a selectivity for 5-HT_{2A} over 5-HT_{2C} of at least about 10-fold. In certain embodiments, a selective 5-HT_{2A} partial agonist is a 5-HT_{2A} partial agonist having a selectivity for 5-HT_{2A} over 5-HT_{2C} of at least about 100-fold.

PHARMACEUTICAL COMPOSITION shall mean a composition comprising at least one active ingredient; including but not limited to, salts, solvates and hydrates of compounds of Formula (I); whereby the composition is amenable to investigation for a specified, efficacious outcome in a mammal (for example and without limitation, a human). Those of ordinary still in the art will understand and appreciate the techniques appropriate for determining whether an active ingredient has a desired efficacious outcome based upon the needs of the artisan.

THERAPEUTICALLY EFFECTIVE AMOUNT as used herein refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that may be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

Where a range of values is provided, it is understood that each intervening value, to the tenth of the lower limit unless the context clearly indicates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### COMPOUNDS OF THE INVENTION

One aspect of the present invention encompasses certain diaryl and arylheteroaryl urea derivatives as shown in Formula (I):

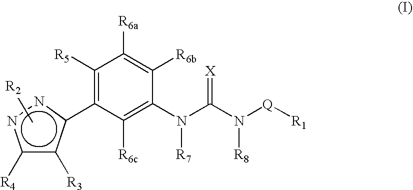

or a pharmaceutically acceptable salt, hydrate or solvate thereof; wherein R₁, R₂, R₃, R₄, R₅, R₆ₐ, R_{6b}, R_{6c}, R₇, R₈, X, and Q have the same definitions as described herein, supra and infra.

Some embodiments of the present invention encompass certain diaryl and arylheteroaryl urea derivatives as shown in the following Formula

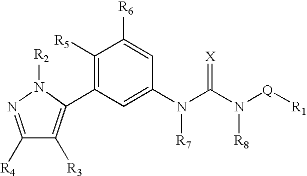

wherein:

i) R₁ is aryl or heteroaryl optionally substituted with R₉, R₁₀, R₁₁, R₁₂, R₁₃, R₁₄, and R₁₅ selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F, Cl, or Br; and wherein each of said C₂₋₆ alkenyl, C₁₋₆ alkyl, C₂₋₆ alkynyl and phenyl groups can be optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;

ii) R₂ is selected from the group consisting of C₁₋₆ alkyl, C₂₋₆ alkenyl, C₂₋₆ alkynyl and C₃₋₇ cycloalkyl;

iii) R₃ is selected from the group consisting of H, C₂₋₆ alkenyl, C₁₋₆ alkyl, C₁₋₆ alkylcarboxamide, C₂₋₆ alkynyl, C₁₋₆ alkylsulfonamide, carbo-C₁₋₆-alkoxy, carboxamide, carboxy, cyano, C₃₋₇ cycloalkyl, C₂₋₈ dialkylcarboxamide, halogen, heteroaryl and phenyl; and wherein each of said C₂₋₆ alkenyl, C₁₋₆ alkyl, C₂₋₆ alkynyl, C₁₋₆ alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and sulfonamide;

iv) R₄ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

v) R₅ is selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide, wherein said C₁₋₆ alkoxy group can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and phenyl, and wherein said phenyl is optionally substituted with 1 to 5 halogen atoms;

vi) R₆ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

vii) R₇ and R₈ are independently H or C₁₋₈ alkyl;

viii) X is O or S; and

ix) Q is C₁₋₃ alkylene optionally substituted with 1 to 4 substituents selected from the group consisting of C₁₋₃ alkyl, C₁₋₄ alkoxy, carboxy, cyano, C₁₋₃ haloalkyl, halogen and oxo; or Q is a bond; or a pharmaceutically acceptable salt, hydrate or solvate thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

Compounds of the invention can also include tautomeric forms, such as keto-enol tautomers, and the like. Tautomeric forms can be in equilibrium or sterically locked into one form by appropriate substitution. It is understood that the various tautomeric forms are within the scope of the compounds of the present invention.

Compounds of the invention can also include all isotopes of atoms occurring in the intermediates and/or final compounds. Isotopes include those atoms having the same atomic number but different mass numbers. For example, isotopes of hydrogen include deuterium and tritium.

It is understood and appreciated that compounds of the present invention may have one or more chiral centers, and therefore can exist as enantiomers and/or diastereomers. The invention is understood to extend to and embrace all such enantiomers, diastereomers and mixtures thereof, including but not limited, to racemates. Accordingly, some embodiments of the present invention pertain to compounds of the present invention that are R enantiomers. Further, some embodiments of the present invention pertain to compounds of the present invention that are S enantiomers. In examples where more than one chiral center is present, then, some embodiments of the present invention include compounds that are RS or SR enantiomers. In further embodiments, compounds of the present invention are RR or SS enantiomers. It is understood that compounds of the present invention are intended to represent all individual enantiomers and mixtures thereof, unless stated or shown otherwise.

In some embodiments, R₁ is aryl or heteroaryl each optionally substituted with R₉, R₁₀, R₁₁, R₁₂, R₁₃, R₁₄, and R₁₅ each selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, hydroxyl, thiol, nitro, phenoxy and phenyl, wherein said C₂₋₆ alkenyl, C₁₋₆ alkyl, C₂₋₆ alkynyl, C₁₋₆ alkylamino, C₁₋₆ alkylimino, C₂₋₈ dialkylamino, heterocyclic, and phenyl are each optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;

Some embodiments of the present invention pertain to compounds wherein R₁ is phenyl or naphthyl each optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, carboxamide, cyano, C₃₋₇ cycloalkyl, halogen, C₁₋₆ haloalkoxy, C₁₋₆ haloalkyl, and hydroxyl.

Some embodiments of the present invention pertain to compounds wherein R₁ is phenyl optionally substituted with R₉, R₁₀, R₁₁, R₁₂, and R₁₃ each selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, halogen, C₁₋₆ haloalkoxy, C₁₋₆ haloalkyl, heterocyclic, hydroxyl, nitro, and phenyl, or two adjacent R₉, R₁₀, R₁₁, R₁₂, and R₁₃ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, carboxamide, cyano, C₃₋₇ cycloalkyl, halogen, C₁₋₆ haloalkoxy, C₁₋₆ haloalkyl, and hydroxyl.

Some embodiments of the present invention pertain to compounds wherein R₁ is phenyl or naphthyl each optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

Some embodiments of the present invention pertain to compounds wherein R₁ is phenyl optionally substituted with R₉, R₁₀, R₁₁, R₁₂, and R₁₃ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, and R₁₃ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

Some embodiments of the present invention pertain to compounds wherein R₁ is phenyl or naphthyl optionally substituted with R₉, R₁₀, R₁₁, R₁₂, R₁₃, R₁₄, and R₁₅ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino [i.e., —N(CH₃)CH₂CH₂N(CH₃)₂], (3-dimethylamino-propyl)-methyl-amino [i.e., —N(CH₃)CH₂CH₂CH₂N(CH₃)₂], —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

Some embodiments of the present invention pertain to compounds wherein R₁ is phenyl optionally substituted with R₉, R₁₀, R₁₁, R₁₂, and R₁₃, R₁₄ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino [i.e., —N(CH₃)CH₂CH₂N(CH₃)₂], (3-dimethylamino-propyl)-methyl-amino [i.e., —N(CH₃)CH₂CH₂CH₂N(CH₃)₂], —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

Some embodiments of the present invention pertain to compounds wherein R₁ is phenyl or naphthyl optionally substituted with R₉, R₁₀, R₁₁, R₁₂, R₁₃, R₁₄, and R₁₅ each selected independently from the group consisting of —OCH₃, —CH₃, cyano, —F, —Cl, —Br, —OCF₃, and —CF₃.

Some embodiments of the present invention pertain to compounds wherein R₁ is phenyl optionally substituted with R₉, R₁₀, R₁₁, R₁₂, and R₁₃ each selected independently from the group consisting of —OCH₃, —CH₃, cyano, —F, —Cl, —Br, —OCF₃, and —CF₃.

Some embodiments of the present invention pertain to compounds wherein R₁ is phenyl and can be represented by the Formula shown below:

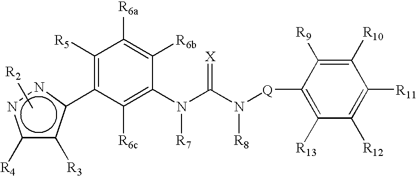

wherein each variable in the above formula has the same meaning as described herein, supra and infra. In some embodiments, R₇ and R₈ are both —H, Q is a bond, and X is O.

Some embodiments of the present invention pertain to compounds wherein R₁ is phenyl and can be represented by Formula (Ia) as shown below:

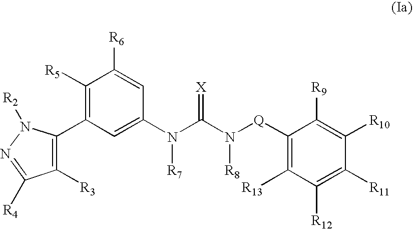

wherein:

R₉ to R₁₃ substituents are each selected independently from the group consisting of H, C₁₋₆ acyl, C₁₋₆ acyloxy, C₁₋₆ alkoxy, C₁₋₆ alkyl, C₁₋₆ alkylcarboxamide, C₁₋₆ alkylsulfonamide, C₁₋₆ alkylsulfinyl, C₁₋₆ alkylsulfonyl, C₁₋₆ alkylthio, amino, C₁₋₆ alkylamino, C₂₋₈ dialkylamino, carbo-C₁₋₆-alkoxy, carboxamide, carboxy, cyano, halogen, C₁₋₆ haloalkoxy, C₁₋₆ haloalkyl, hydroxyl, nitro and phenyl, or two adjacent substituents together with the phenyl form a C₅₋₇ cycloalkyl optionally comprising 1 to 2 oxygen atoms; and wherein each said C₁₋₆ alkyl and phenyl groups can be optionally substituted with 1 to 5 substituents selected independently from the group consisting of C₁₋₆ alkoxy, C₁₋₆ alkyl, amino, cyano, halogen, C₁₋₆ haloalkoxy, C₁₋₆ haloalkyl, hydroxyl and nitro.

In some embodiments, R₁ is phenyl optionally substituted with R₉ to R₁₃ substituents selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl; and wherein said phenyl can be optionally substituted with 1 to 5 substituents selected independently from the group consisting of C₁₋₆ alkoxy, C₁₋₆ alkyl, cyano, halogen, C₁₋₆ haloalkoxy, C₁₋₆ haloalkyl and nitro.

In some embodiments, R₁ is phenyl optionally substituted with R₉ to R₁₃ substituents selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl.

In some embodiments, R₁ is phenyl optionally substituted with R₉ to R₁₃ substituents selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, F, Cl, Br, I, —OCF₃, —OCHF₂, —OCFH₂, —OCF₂CF₃, —OCH₂CF₃, —CF₃, —CHF₂, —CFH₂, —CF₂CF₃, —CH₂CF₃, nitro and phenyl.

In some embodiments, R₁ is phenyl optionally substituted with R₉ to R₁₃ substituents are each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

In some embodiments, R₁ is phenyl optionally substituted with R₉, R₁₀, R₁₁, R₁₂ and R₁₃ substituents selected independently from the group consisting of —C(O)CH₃, —OCH₃, —CH₃, cyano, —F, —Cl, —Br, —OCF₃, —CF₃, nitro and phenyl.

Some embodiments of the present invention pertain to compounds wherein R₁ is naphthyl optionally substituted with R₉ R₁₀ R₁₁ R₁₂ R₁₃ R₁₄ and R₁₅ substituents selected independently from the group consisting of C₁₋₆ acyl, C₁₋₆ acyloxy, C₁₋₆ alkoxy, C₁₋₆ alkyl, C₁₋₆ alkylcarboxamide, C₁₋₆ alkylsulfonamide, C₁₋₆ alkylsulfinyl, C₁₋₆ alkylsulfonyl, C₁₋₆ alkylthio, amino, C₁₋₆ alkylamino, C₂₋₈ dialkylamino, carbo-C₁₋₆-alkoxy, carboxamide, carboxy, cyano, halogen, C₁₋₆ haloalkoxy, C₁₋₆ haloalkyl, hydroxyl and nitro; and wherein said C₁₋₆ alkyl can be optionally substituted with 1 to 5 substituents selected independently from the group consisting of C₁₋₆ alkoxy, C₁₋₆ alkyl, amino, cyano, halogen, C₁₋₆ haloalkoxy, C₁₋₆ haloalkyl, hydroxyl and nitro.

In some embodiments, R₁ is naphthyl optionally substituted with R₉, R₁₀, R₁₁, R₁₂, R₁₃, R₁₄ and R₁₅ substituents selected independently from the group consisting of C₁₋₆ acyl, C₁₋₆ alkoxy, C₁₋₆ alkyl, cyano, halogen, C₁₋₆ haloalkoxy, C₁₋₆ haloalkyl and nitro.

In some embodiments, R₁ is naphthyl optionally substituted with R₉, R₁₀, R₁₁, R₁₂, R₁₃, R₁₄ and R₁₅ substituents selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, —F, —Cl, —Br, —I, —OCF₃, —OCHF₂, —OCFH₂, —OCF₂CF₃, —OCH₂CF₃, —CF₃, —CHF₂, —CFH₂, —CF₂CF₃, —CH₂CF₃ and nitro.

In some embodiments, R₁ is naphthyl optionally substituted with R₉, R₁₀, R₁₁, R₁₂, R₁₃, R₁₄ and R₁₅ substituents selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, —F, —Cl, —Br, —I, —OCF₃, —OCHF₂, —OCFH₂, —OCF₂CF₃, —OCH₂CF₃, —CF₃, —CHF₂, —CFH₂, —CF₂CF₃, —CH₂CF₃ and nitro.

In some embodiments, R₁ is naphthyl optionally substituted with R₉, R₁₀, R₁₁, R₁₂, R₁₃, R₁₄ and R₁₅ substituents selected independently from the group consisting of —C(O)CH₃, —OCH₃, —CH₃, cyano, —F, —Cl, —Br, —OCF₃, —CF₃ and nitro.

Some embodiments of the present invention pertain to compounds wherein R₁ is heteroaryl optionally substituted with R₉, R₁₀, R₁₁, R₁₂, and R₁₃ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

Some embodiments of the present invention pertain to compounds wherein R₁ is heteroaryl optionally substituted with R₉, R₁₀, R₁₁, R₁₂, and R₁₃ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

Some embodiments of the present invention pertain to compounds wherein R₁ is heteroaryl optionally substituted with R₉, R₁₀, R₁₁, R₁₂, and R₁₃ each selected independently from the group consisting of —OCH₃, —CH₃, cyano, —F, —Cl, —Br, —OCF₃, and —CF₃.

Some embodiments of the present invention pertain to compounds wherein R₁ is heteroaryl optionally substituted with R₉, R₁₀, R₁₁, R₁₂, and R₁₃ each selected independently from the group consisting of C₁₋₆ acyl, C₁₋₆ acyloxy, C₁₋₆ alkoxy, C₁₋₆ alkyl, C₁₋₆ alkylcarboxamide, C₁₋₆ alkylsulfonamide, C₁₋₆ alkylsulfinyl, C₁₋₆ alkylsulfonyl, C₁₋₆ alkylthio, amino, C₁₋₆ alkylamino, C₂₋₈ dialkylamino, carbo-C₁₋₆-alkoxy, carboxamide, carboxy, cyano, halogen, C₁₋₆ haloalkoxy, C₁₋₆ haloalkyl, hydroxyl, nitro and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group; and wherein each of said C₁₋₆ alkyl and phenyl groups can be optionally substituted with 1 to 5 substituents selected independently from the group consisting of C₁₋₆ alkoxy, C₁₋₆ alkyl, amino, cyano, halogen, C₁₋₆ haloalkoxy, C₁₋₆ haloalkyl, hydroxyl and nitro.

In some embodiments, R₁ is heteroaryl optionally substituted with R₉, R₁₀, R₁₁, R₁₂ and R₁₃ each selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl; and wherein said phenyl can be optionally substituted with 1 to 5 substituents selected independently from the group consisting of C₁₋₆ alkoxy, C₁₋₆ alkyl, cyano, halogen, C₁₋₆ haloalkoxy, C₁₋₆ haloalkyl and nitro.

In some embodiments, R₁ is heteroaryl optionally substituted with R₉, R₁₀, R₁₁, R₁₂ and R₁₃ each selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl.

In some embodiments, R₁ is heteroaryl optionally substituted with R₉, R₁₀, R₁₁, R₁₂, and R₁₃ each selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, —F, —Cl, —Br, —I, —OCF₃, —OCHF₂, —OCFH₂, —OCF₂CF₃, —OCH₂CF₃, —CF₃, —CHF₂, —CFH₂, —CF₂CF₃, —CH₂CF₃, nitro and phenyl.

In some embodiments, R₁ is heteroaryl optionally substituted with R₉, R₁₀, R₁₁, R₁₂, and R₁₃ each selected independently from the group consisting of —C(O)CH₃, —OCH₃, —CH₃, cyano, —F, —Cl, —Br, —OCF₃, —CF₃, nitro and phenyl. In some embodiments, R₁ is heteroaryl optionally substituted with R₉, R₁₀, R₁₁, R₁₂, and R₁₃ selected independently from the group consisting of H, —C(O)CH₃, —OCH₃, —CH₃, cyano, —F, —Cl, —Br, —OCF₃, —CF₃, nitro and phenyl.

In some embodiments R₁ is heteroaryl having 5-atoms in the aromatic ring examples of which are represented by the following formulae:

TABLE 2

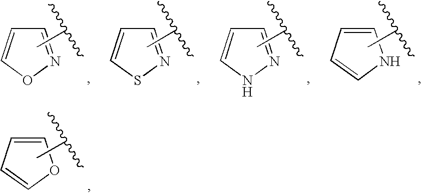

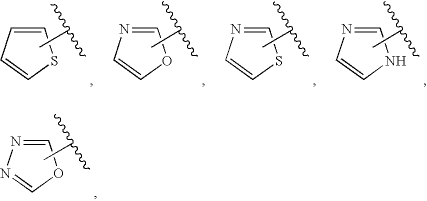

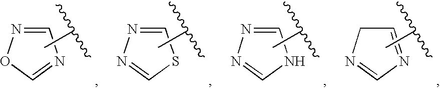

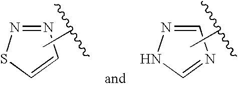

wherein the 5-membered heteroaryl is bonded at any available position of the ring, for example, a imidazolyl ring can be bonded at one of the ring nitrogens (i.e., imidazol-1-yl group) or at one of the ring carbons (i.e., imidazol-2-yl, imidazol-4-yl or imidazol-5-yl group).

In some embodiments, R₁ is a 6-membered heteroaryl, for example, a 6-membered heteroaryl as shown in TABLE 3:

TABLE 3

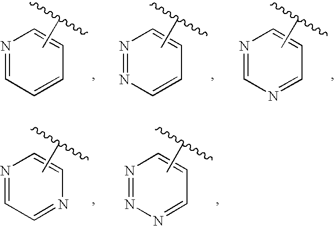

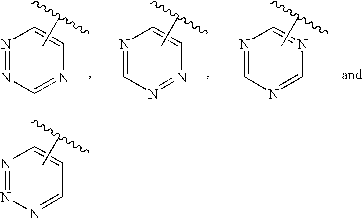

wherein the heteroaryl group is bonded at any ring carbon. In some embodiments, R₁ is selected from the group consisting of pyridinyl, pyridazinyl, pyrimidinyl and pyrazinyl. In some embodiments, R₁ is pyridinyl.

In some embodiments R₁ is a heteroaryl, for example but not limited to those shown in TABLE 2 and 3, optionally substituted with 1 to 3 substituents selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro, phenoxy and phenyl; and wherein each of said C₂₋₆ alkenyl, C₁₋₆ alkyl, C₂₋₆ alkynyl and phenyl groups can be optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro.

Some embodiments of the present invention pertain to compounds wherein R₂ is H or C₁₋₆ alkyl.

Some embodiments of the present invention pertain to compounds wherein R₂ is C₁₋₆ alkyl. In some embodiments, R₂ is selected from the group consisting of —CH₃, —CH₂CH₃, —CH(CH₃)₂, —CH₂CH₂CH₃, —CH₂CH(CH₃)₂ and —CH₂CH₂CH₂CH₃. In some embodiments, R₂ is —CH₃ or —CH(CH₃)₂.

Some embodiments of the present invention can be represented by Formulae (Ib) and (Ic) respectively as shown below:

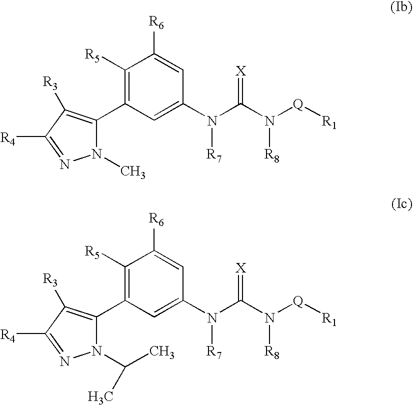

wherein each variable in Formulae (Ib) and (Ic) has the same meaning as described herein, supra and infra.

Some embodiments of the present invention pertain to compounds wherein R₂ is H.

It is understood that when R₂ is H, then tautomers are possible. It is well understood and appreciated in the art that pyrazoles can exist in various tautomeric forms. Two possible tautomeric forms are illustrated below:

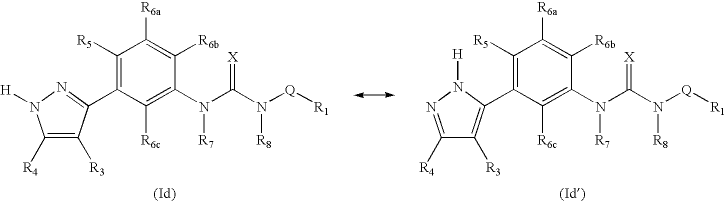

It is further understood that tautomeric forms can also have corresponding nomenclature for each represented tautomer, for example, Formula (Id) and Formula (Id′) can be represented by the general chemical names 1H-pyrazol-3-yl and 2H-pyrazole-3-yl respectively. Therefore, the present invention includes all tautomers and the various nomenclature designations.

Some embodiments of the present invention pertain to compounds wherein R₂ is C₂₋₆ alkenyl. In some embodiments, R₂ is —CH₂CH═CH₂.

Some embodiments of the present invention pertain to compounds wherein R₂ is C₂₋₆ alkynyl.

Some embodiments of the present invention pertain to compounds wherein R₂ is C₃₋₇ cycloalkyl. In some embodiments, R₂ is cyclopropyl.

Some embodiments of the present invention pertain to compounds wherein R₃ is selected from the group consisting of H, C₂₋₆ alkenyl, C₁₋₆ alkyl, C₁₋₆ alkylcarboxamide, C₂₋₆ alkynyl, carbo-C₁₋₆-alkoxy, carboxamide, carboxy, cyano, C₃₋₇ cycloalkyl, halogen, heteroaryl or phenyl; and wherein each of said C₂₋₆ alkenyl, C₁₋₆ alkyl, C₂₋₆ alkynyl, heteroaryl and phenyl groups can be optionally substituted with 1 to 5 substituents selected independently from the group consisting of C₁₋₆ alkylamino, C₂₋₈ dialkylamino, C₂₋₆ alkenyl, C₁₋₄ alkoxy, C₁₋₈ alkyl, C₂₋₆ alkynyl, amino, halogen, C₁₋₄ haloalkoxy and hydroxyl.

In some embodiments, R₃ is selected from the group consisting of H, C₂₋₆ alkenyl, C₁₋₆ alkyl, C₂₋₆ alkynyl, carbo-C₁₋₆-alkoxy, carboxy, cyano, C₃₋₇ cycloalkyl, halogen, heteroaryl or phenyl; and wherein each of said C₂₋₆ alkenyl, C₁₋₆ alkyl, C₂₋₆ alkynyl and phenyl groups can be optionally substituted with 1 to 5 substituents selected independently from the group consisting of C₂₋₈ dialkylamino, C₂₋₆ alkenyl, C₁₋₄ alkoxy, C₂₋₆ alkynyl, halogen, C₁₋₄ haloalkoxy and hydroxyl.

In some embodiments, R₃ is selected from the group consisting of H, —CH═CH₂, —CH₃, —CH₂CH₃, —CH(CH₃)₂, —CH₂CH₂CH₃, —CH₂CH(CH₃)₂, —CH₂CH₂CH₂CH₃, —C≡CH, —C(O)OCH₃, —C(O)OCH₂CH₃, carboxy, cyano, cyclopropyl, F, Cl, Br, I, thiophen-2-yl, thiophen-3-yl, phenyl, —CH₂CH₂N(CH₃)₂, 2-methoxyphenyl, 3-methoxyphenyl, 4-methoxyphenyl, —CH═CH—C≡CH, 4-fluorophenyl, 4-trifluoromethoxyphenyl, —CH₂OH and —CH₂CH₂OH.

Some embodiments of the present invention pertain to compounds wherein R₃ is H or halogen.

In some embodiments, R₃ is H, F, Cl or Br.

Some embodiments of the present invention pertain to compounds of Formula (Ie) as shown below:

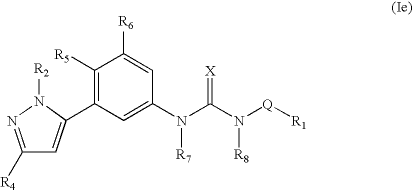

wherein each variable in Formula (Ie) has the same meaning as described herein, supra and infra.

Some embodiments of the present invention pertain to compounds of Formula (If) as shown below:

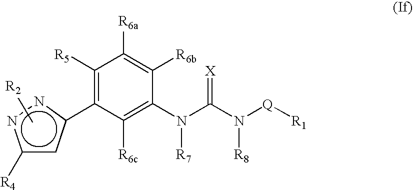

wherein each variable in Formula (If) has the same meaning as described herein, supra and infra.

Some embodiments of the present invention pertain to compounds of Formula (Ig) as shown below:

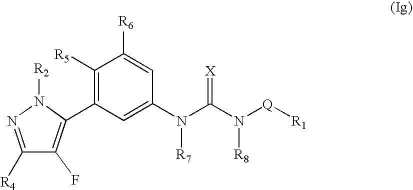

wherein each variable in Formula (Ig) has the same meaning as described herein, supra and infra.

Some embodiments of the present invention pertain to compounds of Formula (Ih) as shown below:

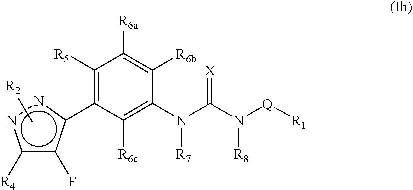

wherein each variable in Formula (Ih) has the same meaning as described herein, supra and infra.

Some embodiments of the present invention pertain to compounds of Formula (Ii) as shown below:

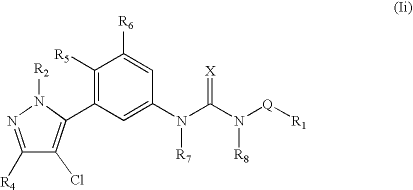

wherein each variable in Formula (Ii) has the same meaning as described herein, supra and infra.

Some embodiments of the present invention pertain to compounds of Formula (Ij) as shown below:

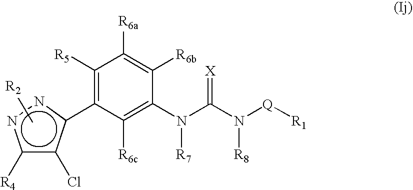

wherein each variable in Formula (Ij) has the same meaning as described herein, supra and infra.

Some embodiments of the present invention pertain to compounds of Formula (Ik) as shown below:

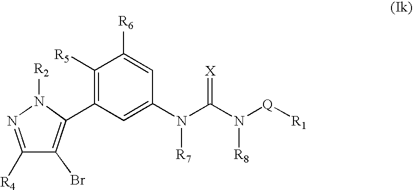

wherein each variable in Formula (Ik) has the same meaning as described herein, supra and infra.

Some embodiments of the present invention pertain to compounds of Formula (Ik′) as shown below:

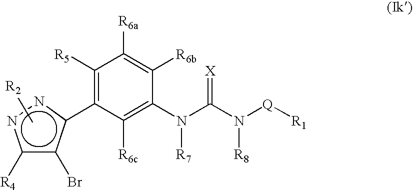

wherein each variable in Formula (Ik′) has the same meaning as described herein, supra and infra.

Some embodiments of the present invention pertain to compounds wherein R₄ is selected from the group consisting of H, C₁₋₆ alkyl and C₁₋₆ haloalkyl.

In some embodiments, R₄ is selected from the group consisting of H, —CH₃, —CH₂CH₃, —CH(CH₃)₂, —CH₂CH₂CH₃, —CH₂CH(CH₃)₂, —CH₂CH₂CH₂CH₃, —CF₃, —CHF₂, —CFH₂, —CF₂CF₃ and —CH₂CF₃.

In some embodiments, R₄ is selected from the group consisting of H or —CF₃.

Some embodiments of the present invention can be represented by Formulae (Im) and (In) as shown below:

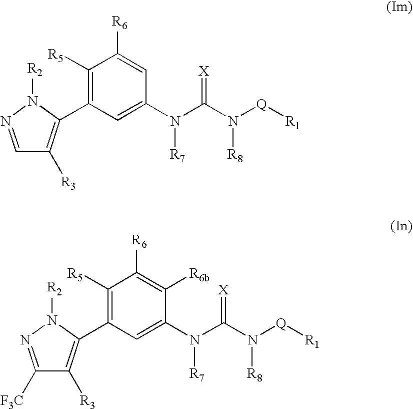

wherein each variable in Formulae (Im) and (In) has the same meaning as described herein, supra and infra.

Some embodiments of the present invention can be represented by Formulae (Io) and (Io′) as shown below:

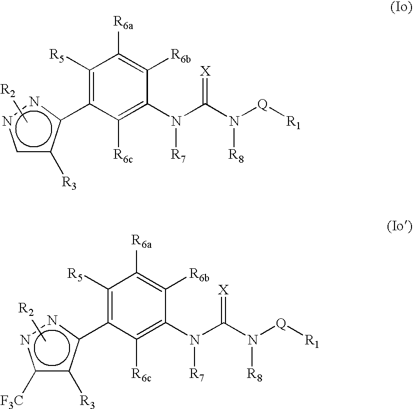

wherein each variable in Formulae (Io) and (Io′) has the same meaning as described herein, supra and infra.

Some embodiments of the present invention pertain to compounds wherein R₅ is selected from the group consisting of C₁₋₆ alkoxy, C₁₋₆ alkylthio, amino, C₁₋₆ alkylamino, C₂₋₈ dialkylamino, halogen, C₁₋₆ haloalkoxy, and hydroxyl, wherein said C₁₋₆ alkoxy group can be optionally substituted with 1 to 5 substituents selected independently from the group consisting of amino, C₁₋₆ alkylamino, C₂₋₈ dialkylamino, amino, carbo-C₁₋₆-alkoxy, carboxamide, carboxy, cyano, halogen, and phenyl, and wherein said amino and phenyl are each optionally substituted with 1 to 5 further substituents selected from the group consisting of halogen and carbo-C₁₋₆-alkoxy.

Some embodiments of the present invention pertain to compounds wherein R₅ is C₁₋₆ alkoxy, or hydroxyl, wherein said C₁₋₆ alkoxy group can be optionally substituted with 1 to 5 substituents selected independently from the group consisting of C₁₋₄ alkoxy, C₁₋₆ alkylamino, C₂₋₈ dialkylamino, alkylsulfinyl, C₁₋₄ alkylsulfonyl, C₁₋₄ alkylthio, amino, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl and phenyl, and wherein said phenyl is optionally substituted with 1 to 5 halogen atoms.

Some embodiments of the present invention pertain to compounds wherein R₅ is selected from the group consisting of C₁₋₆ alkoxy, C₁₋₆ haloalkoxy, and hydroxyl, wherein said C₁₋₆ alkoxy group can be optionally substituted with 1 to 5 substituents selected independently from the group consisting of amino, C₂₋₈ dialkylamino, carboxy, and phenyl, and wherein said amino and phenyl are each optionally substituted with 1 to 5 further substituents selected from the group consisting of halogen and carbo-C₁₋₆-alkoxy.

In some embodiments, R₅ is C₁₋₆ alkoxy, or hydroxyl, and wherein said C₁₋₆ alkoxy group can be optionally substituted with 1 to 5 substituents selected independently from the group consisting of C₁₋₄ alkoxy, C₁₋₆ alkylamino, C₂₋₈ dialkylamino, amino, C₁₋₄ haloalkoxy, hydroxyl and phenyl, wherein said phenyl is optionally substituted with 1 to 5 halogen atoms.

Some embodiments of the present invention pertain to compounds wherein R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy [i.e., —OCH₂CH₂N(CH₃)₂], 3-dimethylamino-propoxy [i.e., —OCH₂CH₂CH₂N(CH₃)₂], carboxymethoxy [i.e., —OCHC(O)OH], and 2-tert-butoxycarbonylamino-ethoxy [i.e., —OCH₂CH₂NHC(O)OC(CH₃)₃].

In some embodiments, R₅ is selected from the group consisting of —OCH₃, —OCH₂CH₃, —OCH(CH₃)₂, —OCH₂CH₂CH₃, —OCH₂CH(CH₃)₂, hydroxyl, —OCH₂CH₂OH, —OCH₂CH₂OCH₃, —OCH₂CH₂OCH₂CH₃, —OCH₂CH₂OCH(CH₃)₂, —OCH₂CH₂OCH₂CH₂CH₃, —OCH₂CH₂OCH₂CH(CH₃)₂, —OCH₂CH₂NH₂, —OCH₂CH₂NHCH₃, —OCH₂CH₂N(CH₃)₂, —OCH₂CH₂OCF₃, —OCH₂CH₂OCHF₂, —OCH₂CH₂OCFH₂, —OCH₂C₆H₅, —OCH₂CH₂C₆H₅, —OCH₂C₆H₅-o-Cl, —OCH₂C₆H₅-m-Cl and —OCH₂C₆H₅-p-Cl.

In some embodiments, R₅ is selected from the group consisting of —OCH₃, —OCH₂CH₃, —OCH(CH₃)₂, hydroxyl, —OCH₂CH₂N(CH₃)₂, —OCH₂C₆H₅, —OCH₂CH₂C₆H₅ and —OCH₂C₆H₅-p-Cl.

In some embodiments, R₅ is —OCH₃.

Some embodiments of the present invention pertains to compounds wherein R₆ is selected from the group consisting of H, C₁₋₆ alkoxy, carbo-C₁₋₆-alkoxy, carboxamide, carboxy, cyano, halogen and hydroxyl.

In some embodiments, R₆ is H.

Some embodiments of the present invention pertain to compounds wherein R₆ₐ, R_{6b}, and R_{6c} are each independently selected from the group consisting of H, C₁₋₆ alkoxy, C₁₋₆ alkyl, amino, C₁₋₆ alkylamino, C₂₋₈ dialkylamino, cyano, halogen, C₁₋₆ haloalkoxy, C₁₋₆ haloalkyl, hydroxyl, and nitro.

Some embodiments of the present invention pertain to compounds wherein R₆ₐ, R_{6b}, and R_{6c} are each independently selected from the group consisting of H, —OCH₃, —CH₃, —N(CH₃)₂, cyano, —F, —Cl, —Br, —OCF₃, hydroxyl, and nitro.

Some embodiments of the present invention pertain to compounds wherein R₆ₐ, R_{6b}, and R_{6c} are each independently selected from the group consisting of H, C₁₋₆ alkoxy, carbo-C₁₋₆-alkoxy, carboxamide, carboxy, cyano, halogen and hydroxyl.

Some embodiments of the present invention pertain to compounds wherein R₆ₐ, R_{6b}, and R_{6c} are all H.

Some embodiments of the present invention pertain to compounds wherein R₅ is C₁₋₆ alkoxy and R₆ₐ, R_{6b}, and R_{6c} are all H.

In some embodiments, R₅ is —OCH₃.

Some embodiments of the present invention pertain to compounds represented by Formula (Ip) as shown below:

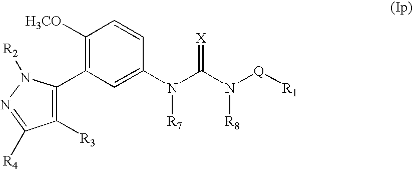

wherein each variable in Formula (Ip) has the same meaning as described herein, supra and infra. In some embodiments, compounds of the present invention have Formula (Ip) and Q is a bond.

Some embodiments of the present invention pertain to compounds represented by Formula (Iq) as shown below:

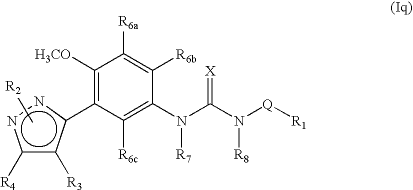

wherein each variable in Formula (Iq) has the same meaning as described herein, supra and infra. In some embodiments, compounds of the present invention have Formula (Iq) and Q is a bond.

Some embodiments of the present invention pertain to compounds wherein R₇ is H or C₁₋₈ alkyl.

In some embodiments, R₇ is selected from the group consisting of H, —CH₃, —CH₂CH₃, —CH(CH₃)₂, —CH₂CH₂CH₃, —CH₂CH(CH₃)₂ and —CH₂CH₂CH₂CH₃.

In some embodiments, R₇ is H.

Some embodiments of the present invention pertain to compounds wherein R₈ is H or C₁₋₈ alkyl.

In some embodiments, R₈ is selected from the group consisting of H, —CH₃, —CH₂CH₃, —CH(CH₃)₂, —CH₂CH₂CH₃, —CH₂CH(CH₃)₂ and —CH₂CH₂CH₂CH₃.

In some embodiments, R₈ is H.

Some embodiments of the present invention pertain to compounds wherein both R₇ and R₈ are H.

Some embodiments of the present invention pertain to compounds represented by Formula (Ir) as shown below:

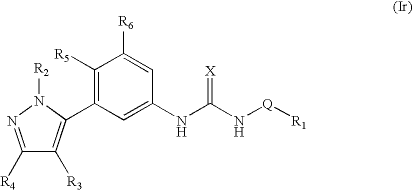

wherein each variable in Formula (Ir) has the same meaning as described herein, supra and infra.

Some embodiments of the present invention pertain to compounds represented by Formula (Is) as shown below:

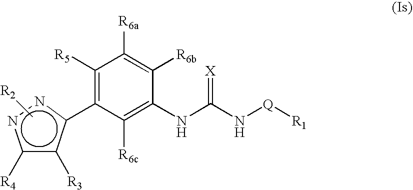

wherein each variable in Formula (Is) has the same meaning as described herein, supra and infra.

Some embodiments of the present invention pertain to compounds wherein X is O (i.e., oxygen).

Some embodiments of the present invention pertain to compounds wherein X is S (i.e., sulfur).

Some embodiments of the present invention pertain to compounds wherein Q is C₁₋₃ alkylene optionally substituted with C₁₋₃ alkyl, C₁₋₃ haloalkyl, halogen and oxo.

Some embodiments of the present invention pertain to compounds wherein Q is a C₁₋₃ alkylene optionally substituted with oxo. As used herein, oxo refers to a double bonded oxygen. In some embodiments, Q is —C(O)— (i.e., a carbonyl).

In some embodiments, Q is —CH₂—.

Some embodiments of the present invention pertain to compounds wherein Q is a bond.

Some embodiments of the present invention can be represented by Formula (It) as shown below:

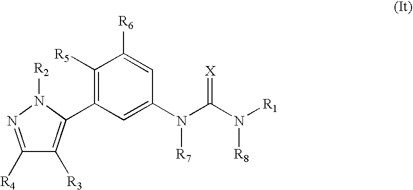

wherein each variable in Formula (It) has the same meaning as described herein, supra and infra.

Some embodiments of the present invention can be represented by Formula (Iu) as shown below:

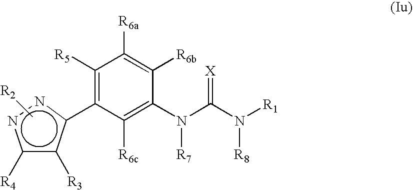

wherein each variable in Formula (Iu) has the same meaning as described herein, supra and infra.

In some embodiments, R₁ is phenyl and can be represented by Formula (Iv) as shown below:

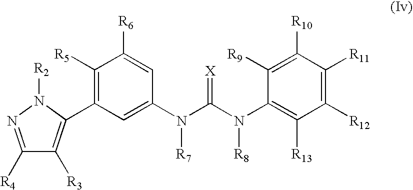

wherein each variable in Formula (Iv) has the same meaning as described herein, supra and infra. In some embodiments, R₇ and R₈ are both H. In some embodiments, X is O (i.e., oxygen).

In some embodiments, R₁ is phenyl and can be represented by Formula (Iw) as shown below:

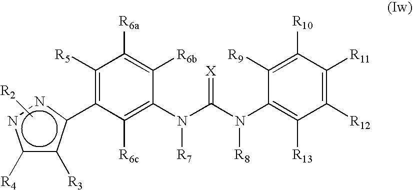

wherein each variable in Formula (Iw) has the same meaning as described herein, supra and infra. In some embodiments, R₇ and R₈ are both H. In some embodiments, X is O (i.e., oxygen).

Some embodiments of the present invention pertain to compounds of Formula (IIa):

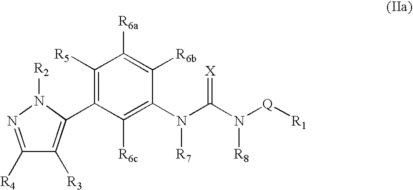

wherein:

R₁ is phenyl or naphthyl optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl;

R₂ is C₁₋₆ alkyl;

R₃ is H or halogen;

R₄ is selected from the group consisting of H, C₁₋₆ alkyl and C₁₋₆ haloalkyl;

R₅ is selected from the group consisting of C₁₋₆ alkoxy, C₁₋₆ haloalkoxy, and hydroxyl, wherein said C₁₋₆ alkoxy group can be optionally substituted with 1 to 5 substituents selected independently from the group consisting of amino, C₂₋₈ dialkylamino, carboxy, and phenyl, and wherein said amino and phenyl are each optionally substituted with 1 to 5 further substituents selected from the group consisting of halogen and carbo-C₁₋₆-alkoxy;

R₆ₐ, R_{6b}, and R_{6c} are each independently selected from the group consisting of H, C₁₋₆ alkoxy, C₁₋₆ alkyl, amino, C₁₋₆ alkylamino, C₂₋₈ dialkylamino, cyano, halogen, C₁₋₆ haloalkoxy, C₁₋₆ haloalkyl, hydroxyl, and nitro

R₇ and R₈ are both H;

X is O; and

Q is a bond.

Some embodiments of the present invention pertain to compounds of Formula (IIa):

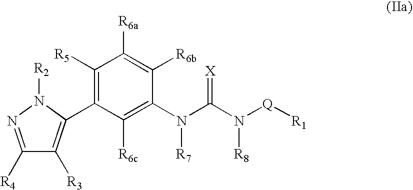

wherein:

R₁ is phenyl or naphthyl optionally substituted with R₉, R₁₀, R₁₁, R₁₂, R₁₃, R₁₄, and R₁₅ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl;

R₂ is —CH₃ or —CH(CH₃)₂;

R₃ is H, F, Cl, or Br;

R₄ is —H, or —CF₃;

R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;

R₆ₐ, R_{6b}, and R_{6c} are each independently selected from the group consisting of H, —OCH₃, —CH₃, —N(CH₃)₂, cyano, —F, —Cl, —Br, —OCF₃, hydroxyl, and nitro;

R₇ and R₈ are both H;

X is O; and

Q is a bond.

Some embodiments of the present invention pertain to compounds of Formula (IIa):

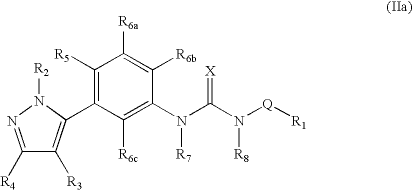

wherein:

R₁ is phenyl optionally substituted with R₉, R₁₀, R₁₁, R₁₂, and R₁₃ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl;

R₂ is —CH₃ or —CH(CH₃)₂;

R₃ is —H, —F, —Cl, or —Br;

R₄ is —H, or —CF₃;

R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;

R₆ₐ, R_{6b}, and R_{6c} are each independently selected from the group consisting of —H, —OCH₃, —CH₃, —N(CH₃)₂, cyano, F, Cl, Br, —OCF₃, hydroxyl, and nitro;

R₇ and R₈ are both H;

X is O; and

Q is a bond.

Some embodiments of the present invention pertain to compounds of Formula (IIa):

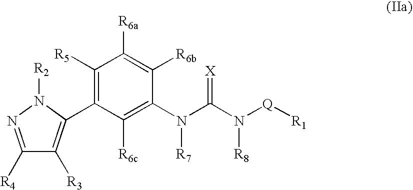

wherein:

R₁ is phenyl optionally substituted with R₉, R₁₀, R₁₁, R₁₂, and R₁₃ each selected independently from the group consisting of —C(O)CH₃, —OCH₃, —CH₃, —CH(CH₃)₂, —N(CH₃)₂, cyano, —F, —Cl, —Br, —OCF₃, —CF₃, hydroxyl, and nitro;

R₂ is —CH₃;

R₃ is —H, —F, —Cl, or —Br;

R₄ is —H;

R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;

R₆ₐ, R_{6b}, and R_{6c} are each —H;

R₇ and R₈ are both —H;

X is O; and

Q is a bond.

Some embodiments of the present invention include compounds illustrated in TABLE A as shown below:

TABLE A

| Cmpd # | Structure | Chemical Name |
|---|---|---|
| 1 | 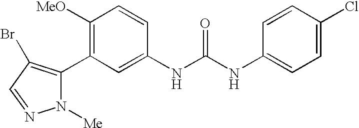 | 1-[3-(4-Bromo-2-methyl-2H-pyrazol-3-yl)-4-methoxy-phenyl]-3-(4-chloro-phenyl)-urea |
| 2 | 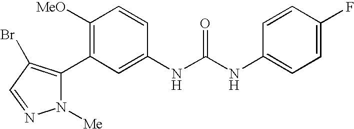 | 1-[3-(4-Bromo-2-methyl-2H-pyrazol-3-yl)-4-methoxy-phenyl]-3-(4-fluoro-phenyl)-urea |
| 3 | 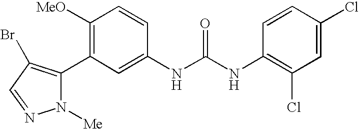 | 1-[3-(4-Bromo-2-methyl-2H-pyrazol-3-yl)-4-methoxy-phenyl]-3-(2,4-dichloro-phenyl)-urea |
| 4 | 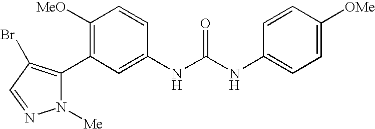 | 1-[3-(4-Bromo-2-methyl-2H-pyrazol-3-yl)-4-methoxy-phenyl]-3-(4-methoxy-phenyl)-urea |
| 5 | 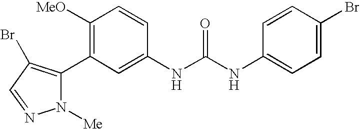 | 1-[3-(4-Bromo-2-methyl-2H-pyrazol-3-yl)-4-methoxy-phenyl]-3-(4-bromo-phenyl)-urea |
| 6 | 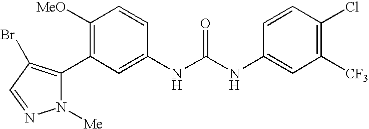 | 1-[3-(4-Bromo-2-methyl-2H-pyrazol-3-yl)-4-methoxy-phenyl]-3-(4-chloro-3-trifluoromethyl-phenyl)-urea |
| 7 | 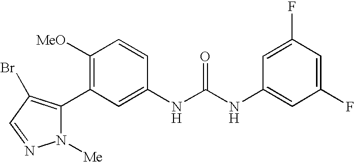 | 1-[3-(4-Bromo-2-methyl-2H-pyrazol-3-yl)-4-methoxy-phenyl]-3-(3,5-difluoro-phenyl)-urea |
| 8 | 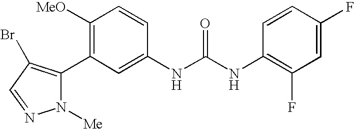 | 1-[3-(4-Bromo-2-methyl-2H-pyrazol-3-yl)-4-methoxy-phenyl]-3-(2,4-difluoro-phenyl)-urea |
| 9 | 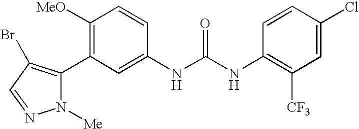 | 1-[3-(4-Bromo-2-methyl-2H-pyrazol-3-yl)-4-methoxy-phenyl]-3-(4-chloro-2-trifluoromethyl-phenyl)-urea |
| 10 | 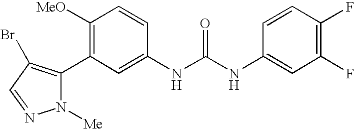 | 1-[3-(4-Bromo-2-methyl-2H-pyrazol-3-yl)-4-methoxy-phenyl]-3-(3,4-difluoro-phenyl)-urea |
| 11 | 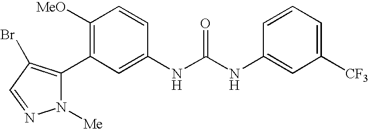 | 1-[3-(4-Bromo-2-methyl-2H-pyrazol-3-yl)-4-methoxy-phenyl]-3-(3-trifluoromethyl-phenyl)-urea |
| 12 | 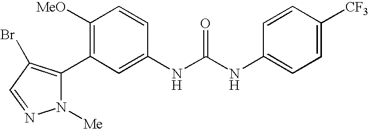 | 1-[3-(4-Bromo-2-methyl-2H-pyrazol-3-yl)-4-methoxy-phenyl]-3-(4-trifluoromethyl-phenyl)-urea |
| 13 | 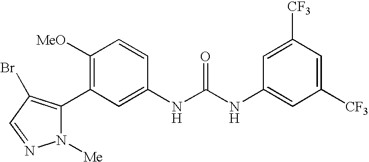 | 1-(3,5-Bis-trifluoromethyl-phenyl)-3-[3-(4-bromo-2-methyl-2H-pyrazol-3-yl)-4-methoxy-phenyl]-urea |
| 14 | 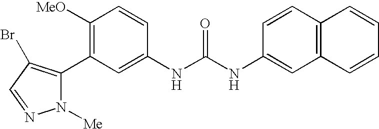 | 1-[3-(4-Bromo-2-methyl-2H-pyrazol-3-yl)-4-methoxy-phenyl]-3-naphthalen-2-yl-urea |
| 15 | 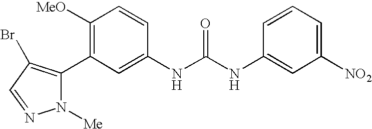 | 1-[3-(4-Bromo-2-methyl-2H-pyrazol-3-yl)-4-methoxy-phenyl]-3-(3-nitro-phenyl)-urea |
| 16 | 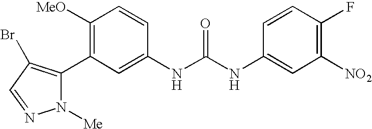 | 1-[3-(4-Bromo-2-methyl-2H-pyrazol-3-yl)-4-methoxy-phenyl]-3-(4-fluoro-3-nitro-phenyl)-urea |
| 17 | 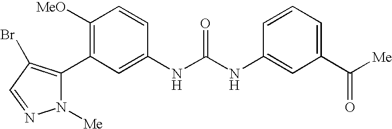 | 1-(3-Acetyl-phenyl)-3-[3-(4-bromo-2-methyl-2H-pyrazol-3-yl)-4-methoxy-phenyl]-urea |
| 18 | 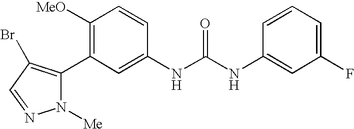 | 1-[3-(4-Bromo-2-methyl-2H-pyrazol-3-yl)-4-methoxy-phenyl]-3-(3-fluoro-phenyl)-urea |
| 19 | 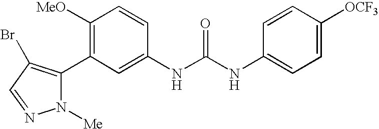 | 1-[3-(4-Bromo-2-methyl-2H-pyrazol-3-yl)-4-methoxy-phenyl]-3-(4-trifluoromethoxy-phenyl)-urea |
| 20 | 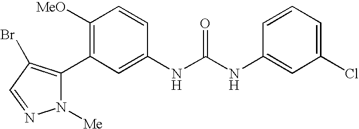 | 1-[3-(4-Bromo-2-methyl-2H-pyrazol-3-yl)-4-methoxy-phenyl]-3-(3-chloro-phenyl)-urea |
| 21 | 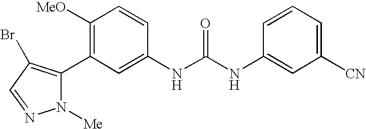 | 1-[3-(4-Bromo-2-methyl-2H-pyrazol-3-yl)-4-methoxy-phenyl]-3-(3-cyano-phenyl)-urea |
| 22 | 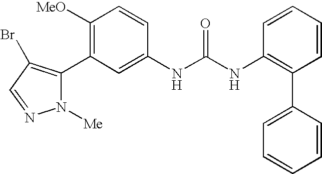 | 1-Biphenyl-2-yl-3-[3-(4-bromo-2-methyl-2H-pyrazol-3-yl)-4-methoxy-phenyl]-urea |
| 23 | 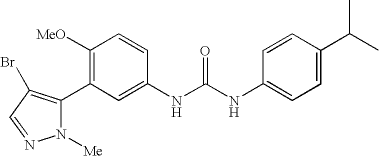 | 1-[3-(4-Bromo-2-methyl-2H-pyrazol-3-yl)-4-methoxy-phenyl]-3-(4-isopropyl-phenyl)-urea |
| 24 | 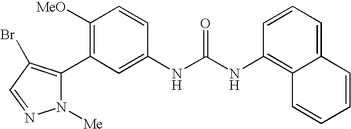 | 1-[3-(4-Bromo-2-methyl-2H-pyrazol-3-yl)-4-methoxy-phenyl]-3-naphthalen-1-yl-urea |
| 25 | 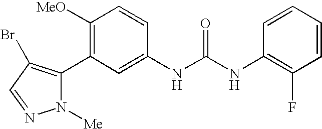 | 1-[3-(4-Bromo-2-methyl-2H-pyrazol-3-yl)-4-methoxy-phenyl]-3-(2-fluoro-phenyl)-urea |
| 26 | 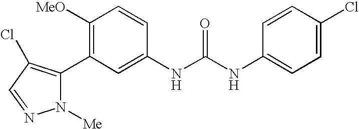 | 1-[3-(4-Chloro-2-methyl-2H-pyrazol-3-yl)-4-methoxy-phenyl]-3-(4-chloro-phenyl)-urea |
| 27 | 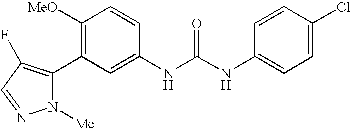 | 1-(4-Chloro-phenyl)-3-[3-(4-fluoro-2-methyl-2H-pyrazol-3-yl)-4-methoxy-phenyl]-urea |
| 28 | 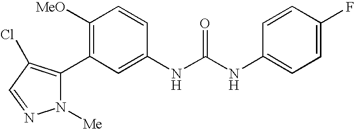 | 1-[3-(4-Chloro-2-methyl-2H-pyrazol-3-yl)-4-methoxy-phenyl]-3-(4-fluoro-phenyl)-urea |
| 29 | 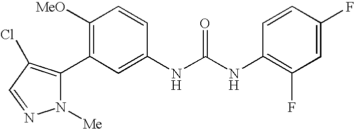 | 1-[3-(4-Chloro-2-methyl-2H-pyrazol-3-yl)-4-methoxy-phenyl]-3-(2,4-difluoro-phenyl)-urea |
| 30 | 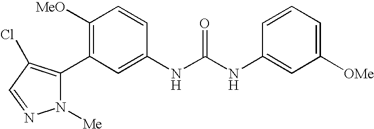 | 1-[3-(4-Chloro-2-methyl-2H-pyrazol-3-yl)-4-methoxy-phenyl]-3-(3-methoxy-phenyl)-urea |
| 31 | 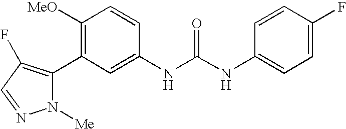 | 1-[3-(4-Fluoro-2-methyl-2H-pyrazol-3-yl)-4-methoxy-phenyl]-3-(4-fluoro-phenyl)-urea |
| 32 | 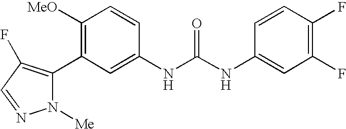 | 1-(3,4-Difluoro-phenyl)-3-[3-(4-fluoro-2-methyl-2H-pyrazol-3-yl)-4-methoxy-phenyl]-urea |
| 33 | 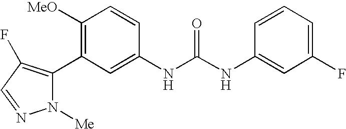 | 1-[3-(4-Fluoro-2-methyl-2H-pyrazol-3-yl)-4-methoxy-phenyl]-3-(3-fluoro-phenyl)-urea |
| 34 | 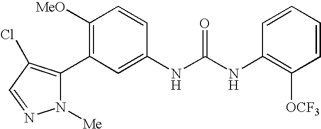 | 1-[3-(4-Chloro-2-methyl-2H-pyrazol-3-yl)-4-methoxy-phenyl]-3-(2-trifluoromethoxy-phenyl)-urea |
| 35 | 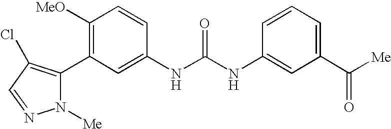 | 1-(3-Acetyl-phenyl)-3-[3-(4-chloro-2-methyl-2H-pyrazol-3-yl)-4-methoxy-phenyl]-urea |
| 36 | 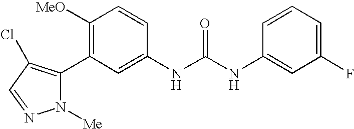 | 1-[3-(4-Chloro-2-methyl-2H-pyrazol-3-yl)-4-methoxy-phenyl]-3-(3-fluoro-phenyl)-urea |
| 37 | 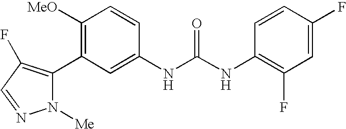 | 1-(2,4-Difluoro-phenyl)-3-[3-(4-fluoro-2-methyl-2H-pyrazol-3-yl)-4-methoxy-phenyl]-urea |
| 38 | 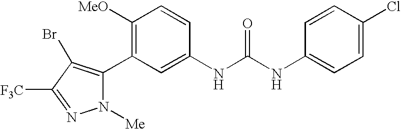 | 1-[3-(4-Bromo-2-methyl-5-trifluoromethyl-2H-pyrazol-3-yl)-4-methoxy-phenyl]-3-(4-chloro-phenyl)-urea |
| 39 | 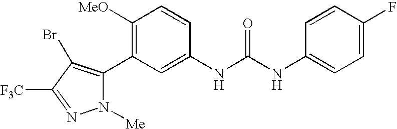 | 1-[3-(4-Bromo-2-methyl-5-trifluoromethyl-2H-pyrazol-3-yl)-4-methoxy-phenyl]-3-(4-fluoro-phenyl)-urea |
| 40 | 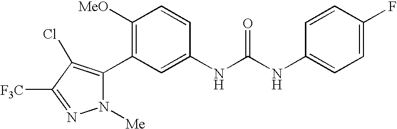 | 1-[3-(4-Chloro-2-methyl-5-trifluoromethyl-2H-pyrazol-3-yl)-4-methoxy-phenyl]-3-(4-fluoro-phenyl)-urea |
| 41 | 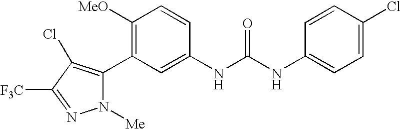 | 1-[3-(4-Chloro-2-methyl-5-trifluoromethyl-2H-pyrazol-3-yl)-4-methoxy-phenyl]-3-(4-chloro-phenyl)-urea |
| 42 | 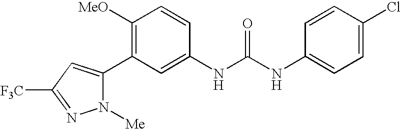 | 1-(4-Chloro-phenyl)-3-[4-methoxy-3-(2-methyl-5-trifluoromethyl-2H-pyrazol-3-yl)-phenyl]-urea |
| 43 | 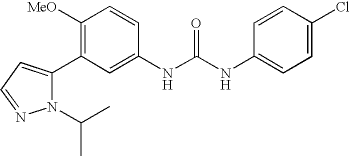 | 1-(4-Chloro-phenyl)-3-[3-(2-isopropyl-2H-pyrazol-3-yl)-4-methoxy-phenyl]-urea |
| 44 | 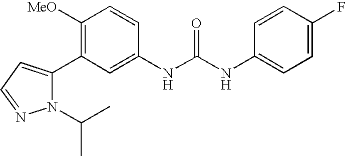 | 1-(4-Fluoro-phenyl)-3-[3-(2-isopropyl-2H-pyrazol-3-yl)-4-methoxy-phenyl]-urea |
| 45 | 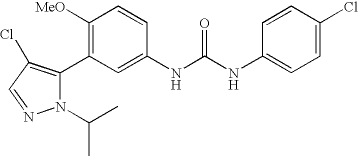 | 1-[3-(4-Chloro-2-isopropyl-2H-pyrazol-3-yl)-4-methoxy-phenyl]-3-(4-chloro-phenyl)-urea |
| 46 | 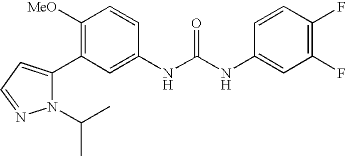 | 1-(3,4-Difluoro-phenyl)-3-[3-(2-isopropyl-2H-pyrazol-3-yl)-4-methoxy-phenyl]-urea |
| 47 | 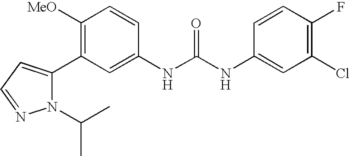 | 1-(3-Chloro-4-fluoro-phenyl)-3-[3-(2-isopropyl-2H-pyrazol-3-yl)-4-methoxy-phenyl]-urea |
| 48 | 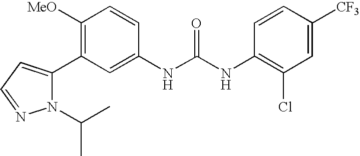 | 1-(2-Chloro-4-trifluoromethyl-phenyl)-3-[3-(2-isopropyl-2H-pyrazol-3-yl)-4-methoxy-phenyl]-urea |
| 49 | 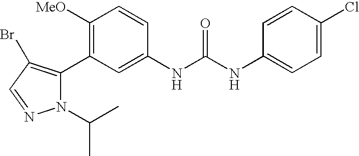 | 1-[3-(4-Bromo-2-isopropyl-2H-pyrazol-3-yl)-4-methoxy-phenyl]-3-(4-chloro-phenyl)-urea |
| 50 | 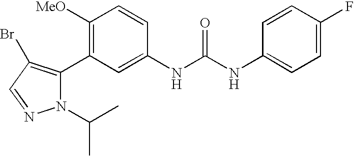 | 1-[3-(4-Bromo-2-isopropyl-2H-pyrazol-3-yl)-4-methoxy-phenyl]-3-(4-fluoro-phenyl)-urea |
| 51 | 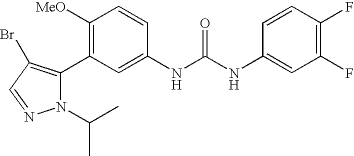 | 1-[3-(4-Bromo-2-isopropyl-2H-pyrazol-3-yl)-4-methoxy-phenyl]-3-(3,4-difluoro-phenyl)-urea |
| 52 | 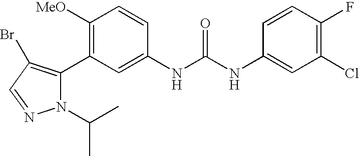 | 1-[3-(4-Bromo-2-isopropyl-2H-pyrazol-3-yl)-4-methoxy-phenyl]-3-(3-chloro-4-fluoro-phenyl)-urea |
| 53 | 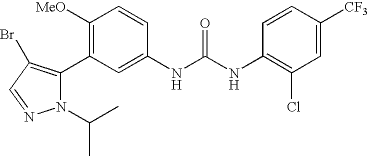 | 1-[3-(4-Bromo-2-isopropyl-2H-pyrazol-3-yl)-4-methoxy-phenyl]-3-(2-Chloro-4-trifluoromethyl-phenyl)-urea |
| 54 | 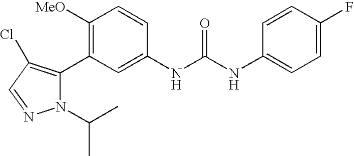 | 1-[3-(4-Chloro-2-isopropyl-2H-pyrazol-3-yl)-4-methoxy-phenyl]-3-(4-fluoro-phenyl)-urea |
| 55 | 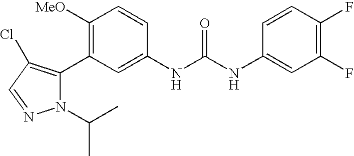 | 1-[3-(4-Chloro-2-isopropyl-2H-pyrazol-3-yl)-4-methoxy-phenyl]-3-(3,4-difluoro-phenyl)-urea |
| 56 | 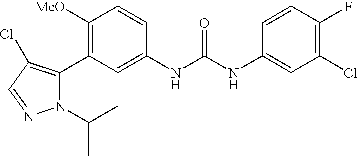 | 1-(3-Chloro-4-fluoro-phenyl)-3-[3-(4-Chloro-2-isopropyl-2H-pyrazol-3-yl)-4-methoxy-phenyl]-urea |
| 57 | 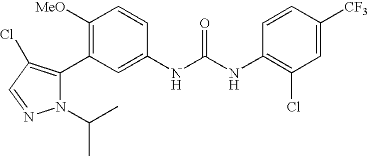 | 1-[3-(4-Chloro-2-isopropyl-2H-pyrazol-3-yl)-4-methoxy-phenyl]-3-(2-Chloro-4-trifluoromethyl-phenyl)-urea |
| 58 | 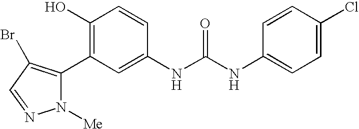 | 1-[3-(4-Bromo-2-methyl-2H-pyrazol-3-yl)-4-hydroxy-phenyl]-3-(4-chloro-phenyl)-urea |
| 59 | 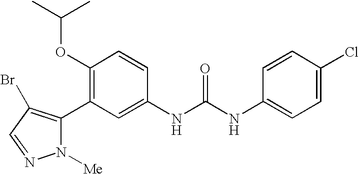 | 1-[3-(4-Bromo-2-methyl-2H-pyrazol-3-yl)-4-isopropoxy-phenyl]-3-(4-chloro-phenyl)-urea |
| 60 | 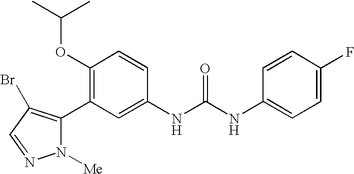 | 1-[3-(4-Bromo-2-methyl-2H-pyrazol-3-yl)-4-isopropoxy-phenyl]-3-(4-fluoro-phenyl)-urea |
| 61 | 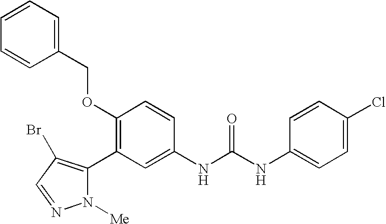 | 1-[4-Benzyloxy-3-(4-bromo-2-methyl-2H-pyrazol-3-yl)-phenyl]-3-(4-chloro-phenyl)-urea |
| 62 | 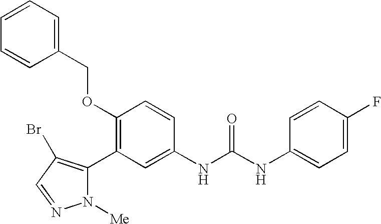 | 1-[4-Benzyloxy-3-(4-bromo-2-methyl-2H-pyrazol-3-yl)-phenyl]-3-(4-fluoro-phenyl)-urea |
| 63 | 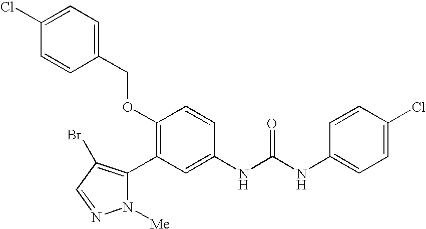 | 1-[3-(4-Bromo-2-methyl-2H-pyrazol-3-yl)-4-(4-chloro-benzyloxy)-phenyl]-3-(4-chloro-phenyl)-urea |
| 64 | 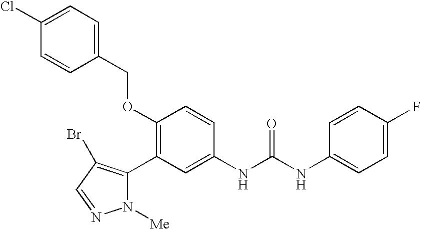 | 1-[3-(4-Bromo-2-methyl-2H-pyrazol-3-yl)-4-(4-chloro-benzyloxy)-phenyl]-3-(4-fluoro-phenyl)-urea |
| 65 | 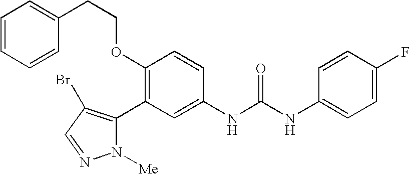 | 1-[3-(4-Bromo-2-methyl-2H-pyrazol-3-yl)-4-phenethyloxy-phenyl]-3-(4-fluoro-phenyl)-urea |
| 66 | 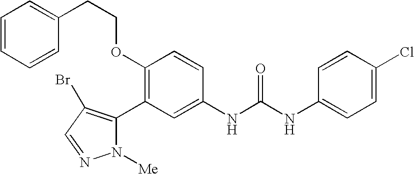 | 1-[3-(4-Bromo-2-methyl-2H-pyrazol-3-yl)-4-phenethyloxy-phenyl]-3-(4-chloro-phenyl)-urea |
| 67 | 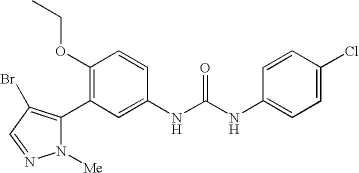 | 1-[3-(4-Bromo-2-methyl-2H-pyrazol-3-yl)-4-ethoxy-phenyl]-3-(4-chloro-phenyl)-urea |
| 68 | 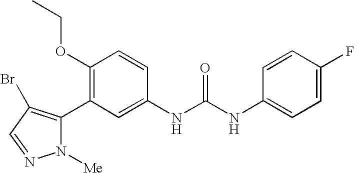 | 1-[3-(4-Bromo-2-methyl-2H-pyrazol-3-yl)-4-ethoxy-phenyl]-3-(4-fluoro-phenyl)-urea |
| 69 | 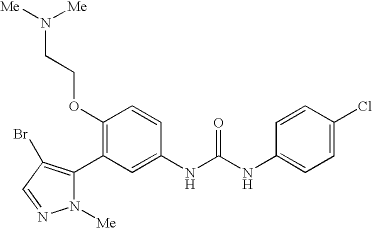 | 1-[3-(4-Bromo-2-methyl-2H-pyrazol-3-yl)-4-(2-dimethylamino-ethoxy)-phenyl]-3-(4-chloro-phenyl)-urea |
| 70 | 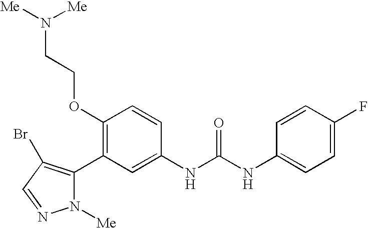 | 1-[3-(4-Bromo-2-methyl-2H-pyrazol-3-yl)-4-(2-dimethylamino-ethoxy)-phenyl]-3-(4-fluoro-phenyl)-urea |
| 71 | 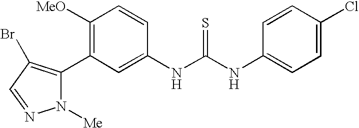 | 1-[3-(4-Bromo-2-methyl-2H-pyrazol-3-yl)-4-methoxy-phenyl]-3-(4-chloro-phenyl)-thiourea |
| 72 | 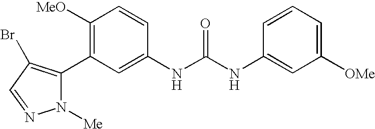 | 1-[3-(4-Bromo-2-methyl-2H-pyrazol-3-yl)-4-methoxy-phenyl]-3-(3-methoxy-phenyl)-urea |
| 73 | 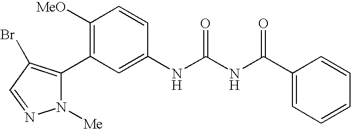 | 1-Benzoyl-3-[3-(4-bromo-2-methyl-2H-pyrazol-3-yl)-4-methoxy-phenyl]-urea |
| 74 | 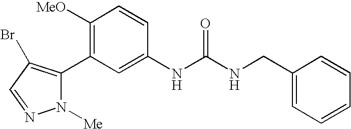 | 1-Benzyl-3-[3-(4-bromo-2-methyl-2H-pyrazol-3-yl)-4-methoxy-phenyl]-urea |
| 75 | 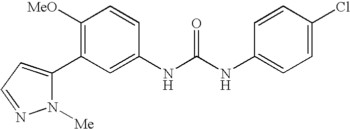 | 1-(4-Chloro-phenyl)-3-[4-methoxy-3-(2-methyl-2H-pyrazol-3-yl)-phenyl]-urea |
| 76 | 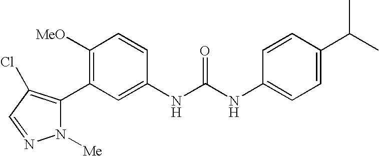 | 1-[3-(4-Chloro-2-methyl-2H-pyrazol-3-yl)-4-methoxy-phenyl]-3-(4-isopropyl-phenyl)-urea |
| 77 | 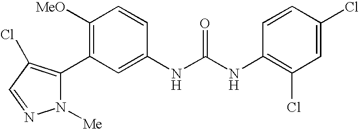 | 1-[3-(4-Chloro-2-methyl-2H-pyrazol-3-yl)-4-methoxy-phenyl]-3-(2,4-dichloro-phenyl)-urea |
| 78 | 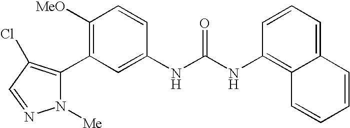 | 1-[3-(4-Chloro-2-methyl-2H-pyrazol-3-yl)-4-methoxy-phenyl]-3-naphthalen-1-yl-urea |
| 79 | 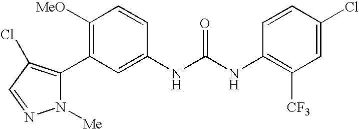 | 1-[3-(4-Chloro-2-methyl-2H-pyrazol-3-yl)-4-methoxy-phenyl]-3-(4-chloro-2-trifluoromethyl-phenyl)-urea |
| 80 | 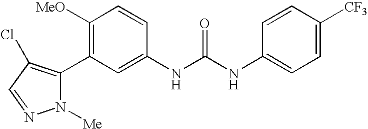 | 1-[3-(4-Chloro-2-methyl-2H-pyrazol-3-yl)-4-methoxy-phenyl]-3-(4-trifluoromethyl-phenyl)-urea |
| 81 | 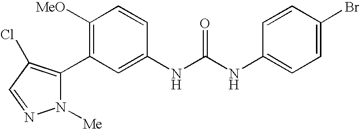 | 1-(4-Bromo-phenyl)-3-[3-(4-chloro-2-methyl-2H-pyrazol-3-yl)-4-methoxy-phenyl]-urea |
| 82 | 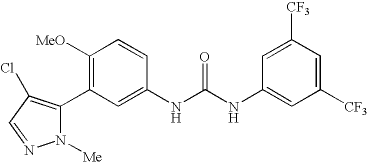 | 1-(3,5-Bis-trifluoromethyl-phenyl)-3-[3-(4-chloro-2-methyl-2H-pyrazol-3-yl)-4-methoxy-phenyl]-urea |
| 83 | 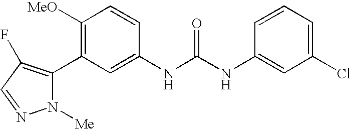 | 1-(3-Chloro-phenyl)-3-[3-(4-fluoro-2-methyl-2H-pyrazol-3-yl)-4-methoxy-phenyl]-urea |
| 84 | 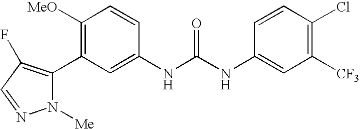 | 1-(4-Chloro-3-trifluoromethyl-phenyl)-3-[3-(4-fluoro-2-methyl-2H-pyrazol-3-yl)-4-methoxy-phenyl]-urea |
| 85 | 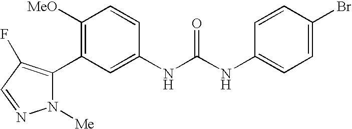 | 1-(4-Bromo-phenyl)-3-[3-(4-fluoro-2-methyl-2H-pyrazol-3-yl)-4-methoxy-phenyl]-urea |
| 86 | 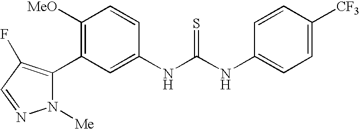 | 1-[3-(4-Fluoro-2-methyl-2H-pyrazol-3-yl)-4-methoxy-phenyl]-3-(4-trifluoromethyl-phenyl)-thiourea |
| 87 | 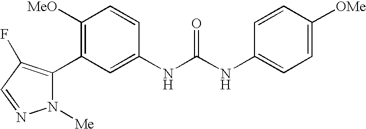 | 1-[3-(4-Fluoro-2-methyl-2H-pyrazol-3-yl)-4-methoxy-phenyl]-3-(4-methoxy-phenyl)-urea |
| 88 | 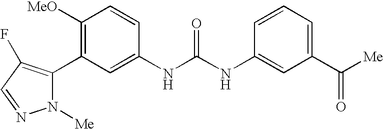 | 1-(3-Acetyl-phenyl)-3-[3-(4-fluoro-2-methyl-2H-pyrazol-3-yl)-4-methoxy-phenyl]-urea |
| 89 | 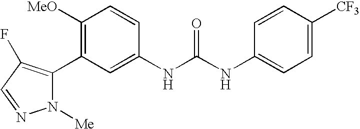 | 1-[3-(4-Fluoro-2-methyl-2H-pyrazol-3-yl)-4-methoxy-phenyl]-3-(4-trifluoromethyl-phenyl)-urea |
| 90 | 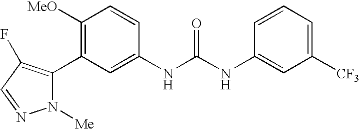 | 1-[3-(4-Fluoro-2-methyl-2H-pyrazol-3-yl)-4-methoxy-phenyl]-3-(3-trifluoromethyl-phenyl)-urea |
| 91 | 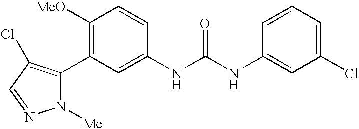 | 1-[3-(4-Chloro-2-methyl-2H-pyrazol-3-yl)-4-methoxy-phenyl]-3-(3-chloro-phenyl)-urea |
| 92 | 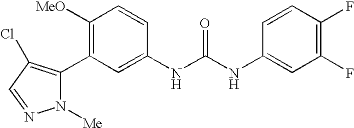 | 1-[3-(4-Chloro-2-methyl-2H-pyrazol-3-yl)-4-methoxy-phenyl]-3-(3,4-difluoro-phenyl)-urea |
| 93 | 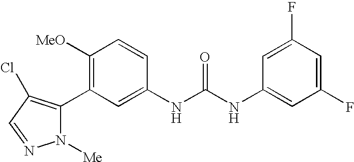 | 1-[3-(4-Chloro-2-methyl-2H-pyrazol-3-yl)-4-methoxy-phenyl]-3-(3,5-difluoro-phenyl)-urea |
| 94 | 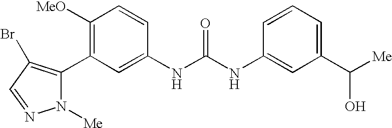 | 1-[3-(4-Bromo-2-methyl-2H-pyrazol-3-yl)-4-methoxy-phenyl]-3-[3-(1-hydroxy-ethyl)-phenyl]-urea |
| 95 | 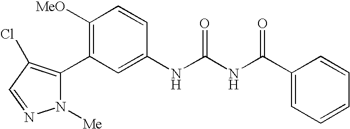 | 1-Benzoyl-3-[3-(4-chloro-2-methyl-2H-pyrazol-3-yl)-4-methoxy-phenyl]-urea |
| 96 | 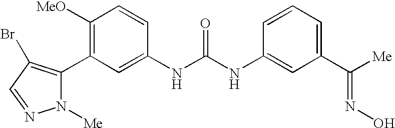 | 1-[3-(4-Bromo-2-methyl-2H-pyrazol-3-yl)-4-methoxy-phenyl]-3-[3-(1-hydroxyimino-ethyl)-phenyl]-urea |
| 97 | 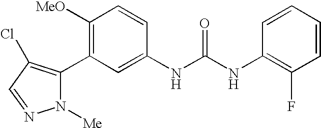 | 1-[3-(4-Chloro-2-methyl-2H-pyrazol-3-yl)-4-methoxy-phenyl]-3-(2-fluoro-phenyl)-urea |
| 98 | 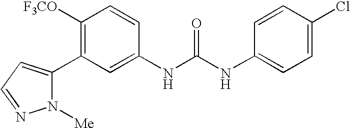 | 1-(4-Chloro-phenyl)-3-[3-(2-methyl-2H-pyrazol-3-yl)-4-trifluoromethoxy-phenyl]-urea |
| 99 | 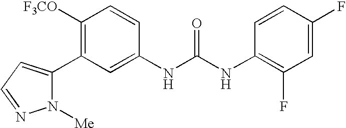 | 1-(2,4-Difluoro-phenyl)-3-[3-(2-methyl-2H-pyrazol-3-yl)-4-trifluoromethoxy-phenyl]-urea |
| 100 | 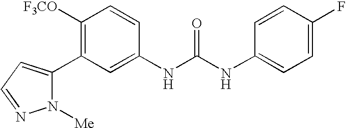 | 1-(4-Fluoro-phenyl)-3-[3-(2-methyl-2H-pyrazol-3-yl)-4-trifluoromethoxy-phenyl]-urea |
| 101 | 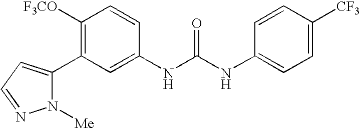 | 1-[3-(2-Methyl-2H-pyrazol-3-yl)-4-trifluoromethoxy-phenyl]-3-(4-trifluoromethyl-phenyl)-urea |
| 102 | 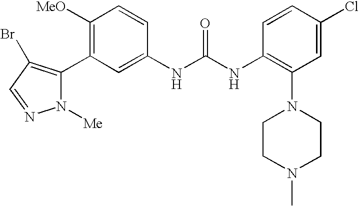 | 1-[3-(4-Bromo-2-methyl-2H-pyrazol-3-yl)-4-methoxy-phenyl]-3-[4-chloro-2-(4-methyl-piperazin-1-yl)-phenyl]-urea |
| 103 | 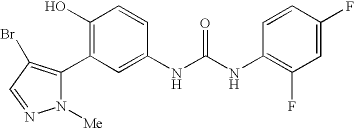 | 1-[3-(4-Bromo-2-methyl-2H-pyrazol-3-yl)-4-hydroxy-phenyl]-3-(2,4-difluoro-phenyl)-urea |
| 104 | 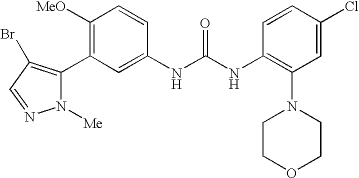 | 1-[3-(4-Bromo-2-methyl-2H-pyrazol-3-yl)-4-methoxy-phenyl]-3-(4-chloro-2-morpholin-4-yl-phenyl)-urea |
| 105 | 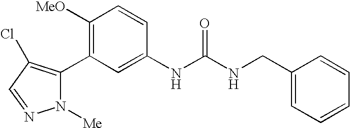 | 1-Benzyl-3-[3-(4-chloro-2-methyl-2H-pyrazol-3-yl)-4-methoxy-phenyl]-urea |
| 106 | 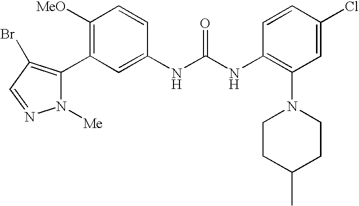 | 1-[3-(4-Bromo-2-methyl-2H-pyrazol-3-yl)-4-methoxy-phenyl]-3-[4-chloro-2-(4-methyl-piperidin-1-yl)-phenyl]-urea |
| 107 | 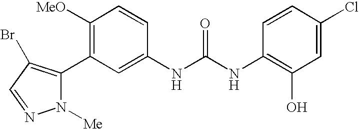 | 1-[3-(4-Bromo-2-methyl-2H-pyrazol-3-yl)-4-methoxy-phenyl]-3-(4-chloro-2-hydroxy-phenyl)-urea |
| 108 | 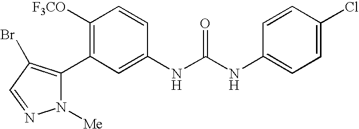 | 1-[3-(4-Bromo-2-methyl-2H-pyrazol-3-yl)-4-trifluoromethoxy-phenyl]-3-(4-chloro-phenyl)-urea |
| 109 | 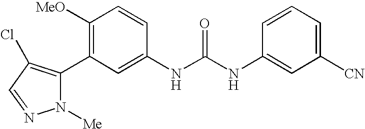 | 1-[3-(4-Chloro-2-methyl-2H-pyrazol-3-yl)-4-methoxy-phenyl]-3-(3-cyano-phenyl)-urea |
| 110 | 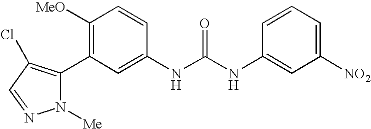 | 1-[3-(4-Chloro-2-methyl-2H-pyraozl-3-yl)-4-methoxy-phenyl]-3-(3-nitro-phenyl)-urea |
| 111 | 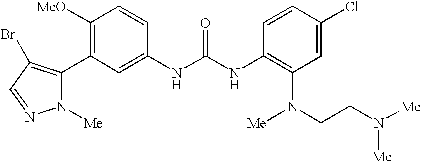 | 1-[3-(4-Bromo-2-methyl-2H-pyrazol-3-yl)-4-methoxy-phenyl]-3-{4-chloro-2-[(2-dimethylamino-ethyl)-methyl-amino]-phenyl}-urea |
| 112 | 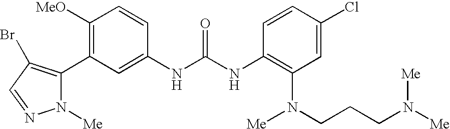 | 1-[3-(4-Bromo-2-methyl-2H-pyrazol-3-yl)-4-methoxy-phenyl]-3-{4-chloro-2-[(3-dimethylamino-propyl)-methyl-amino]-phenyl}-urea |
| 113 | 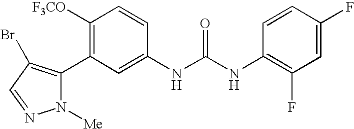 | 1-[3-(4-Bromo-2-methyl-2H-pyrazol-3-yl)-4-trifluoromethoxy-phenyl]-3-(2,4-difluoro-phenyl)-urea |
| 114 | 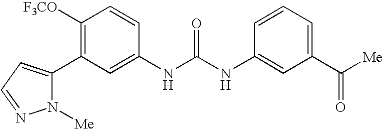 | 1-(3-Acetyl-phenyl)-3-[3-(2-methyl-2H-pyrazol-3-yl)-4-trifluoromethoxy-phenyl]-urea |
| 115 | 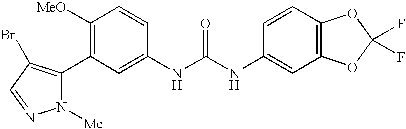 | 1-[3-(4-Bromo-2-methyl-2H-pyrazol-3-yl)-4-methoxy-phenyl]-3-(2,2-difluoro-benzo[1,3]dioxol-5-yl)-urea |
| 116 | 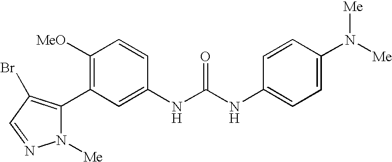 | 1-[3-(4-Bromo-2-methyl-2H-pyrazol-3-yl)-4-methoxy-phenyl]-3-(4-dimethylamino-phenyl)-urea |
| 117 | 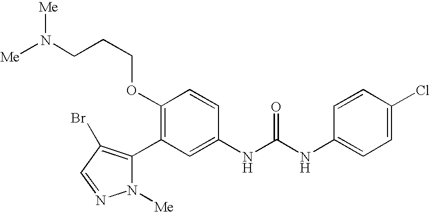 | 1-[3-(4-Bromo-2-methyl-2H-pyrazol-3-yl)-4-(3-dimethylamino-propoxy)-phenyl]-3-(4-chloro-phenyl)-urea |
| 118 | 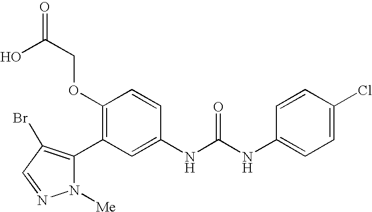 | {2-(4-Bromo-2-methyl-2H-pyrazol-3-yl)-4-[3-(4-chloro-phenyl)-ureido]-phenoxy}-acetic acid |
| 119 | 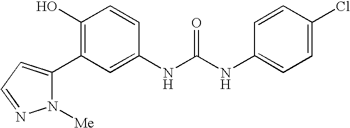 | 1-(4-Chloro-phenyl)-3-[4-hydroxy-3-(2-methyl-2H-pyrazol-3-yl)-phenyl]-urea |
| 120 | 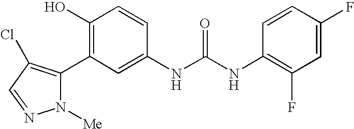 | 1-[3-(4-Chloro-2-methyl-2H-pyrazol-3-yl)-4-hydroxy-phenyl]-3-(2,4-difluoro-phenyl)-urea |
| 121 | 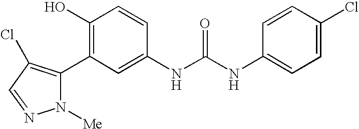 | 1-[3-(4-Chloro-2-methyl-2H-pyrazol-3-yl)-4-hydroxy-phenyl]-3-(4-chloro-phenyl)-urea |
| 122 | 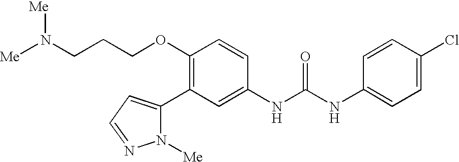 | 1-(4-Chloro-phenyl)-3-[4-(3-dimethylamino-propoxy)-3-(2-methyl-2H-pyrazol-3-yl)-phenyl]-urea |
| 123 | 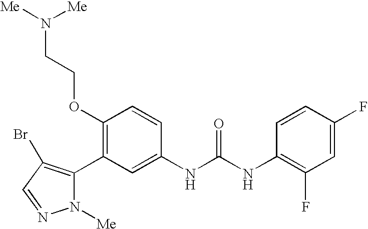 | 1-[3-(4-Bromo-2-methyl-2H-pyrazol-3-yl)-4-(2-dimethylamino-ethoxy)-phenyl]-3-(2,4-difluoro-phenyl)-urea |
| 124 | 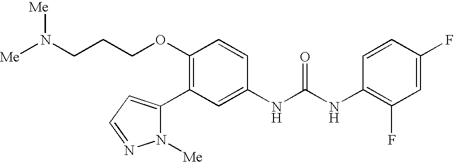 | 1-(2,4-Difluoro-phenyl)-3-[4-(3-dimethylamino-propoxy)-3-(2-methyl-2H-pyrazol-3-yl)-phenyl]-urea |
| 125 | 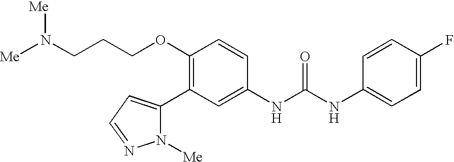 | 1-[4-(3-Dimethylamino-propoxy)-3-(2-methyl-2H-pyrazol-3-yl)-phenyl]-3-(4-fluoro-phenyl)-urea |
| 126 | 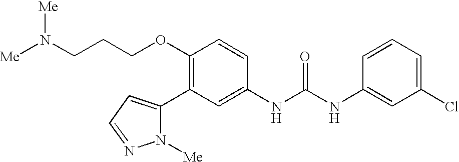 | 1-(4-Chloro-benzyl)-3-[4-(3-dimethylamino-propoxy)-3-(2-methyl-2H-pyrazol-3-yl)-phenyl]-urea |
| 127 | 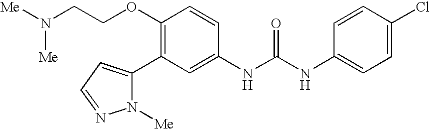 | 1-(4-Chloro-phenyl)-3-[4-(2-dimethylamino-ethoxy)-3-(2-methyl-2H-pyrazol-3-yl)-phenyl]-urea |
| 128 | 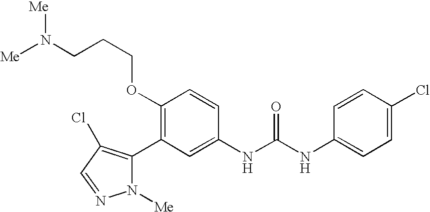 | 1-[3-(4-Chloro-2-methyl-2H-pyrazol-3-yl)-4-(3-dimethylamino-propoxy)-phenyl]-3-(4-chloro-phenyl)-urea |
| 129 | 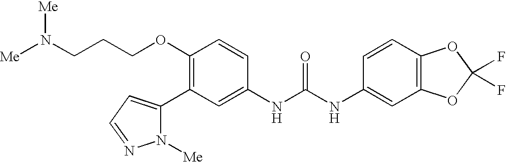 | 1-(2,2-Difluoro-benzo[1,3]dioxol-5-yl)-3-[4-(3-dimethylamino-propoxy)-3-(2-methyl-2H-pyrazol-3-yl)-phenyl]-urea |
| 130 | 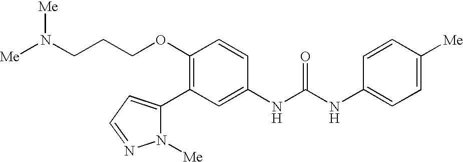 | 1-[4-(3-Dimethylamino-propoxy)-3-(2-methyl-2H-pyrazol-3-yl)-phenyl]-3-p-tolyl-urea |
| 131 | 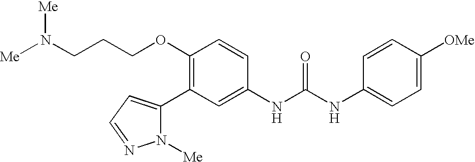 | 1-[4-(3-Dimethylamino-propoxy)-3-(2-methyl-2H-pyrazol-3-yl)-phenyl]-3-(4-methoxy-phenyl)-urea |
| 132 | 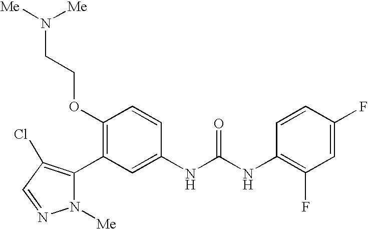 | 1-[3-(4-Chloro-2-methyl-2H-pyrazol-3-yl)-4-(2-dimethylamino-ethoxy)-phenyl]-3-(2,4-difluoro-phenyl)-urea |
| 133 | 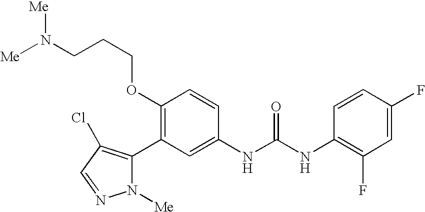 | 1-[3-(4-Chloro-2-methyl-2H-pyrazol-3-yl)-4-(3-dimethylamino-propoxy)-phenyl]-3-(2,4-difluoro-phenyl)-urea |
| 134 | 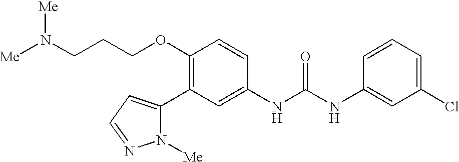 | 1-(3-Chloro-phenyl)-3-[4-(3-dimethylamino-propoxy)-3-(2-methyl-2H-pyrazol-3-yl)-phenyl]-urea |
| 135 | 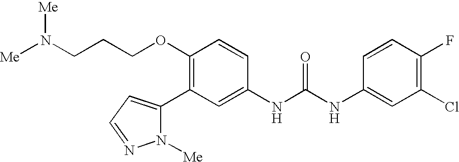 | 1-(3-Chloro-4-fluoro-phenyl)-3-[4-(3-dimethylamino-propoxy)-3-(2-methyl-2H-pyrazol-3-yl)-phenyl]-urea |
| 136 | 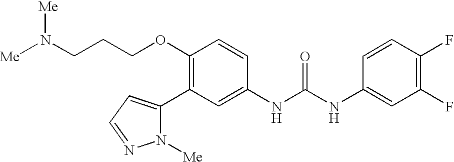 | 1-(3,4-Difluoro-phenyl)-3-[4-(3-dimethylamino-propoxy)-3-(2-methyl-2H-pyrazol-3-yl)-phenyl]-urea |
| 137 | 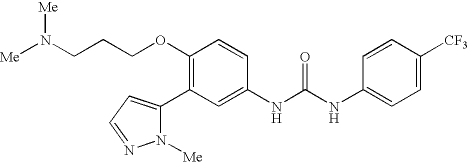 | 1-[4-(3-Dimethylamino-propoxy)-3-(2-methyl-2H-pyrazol-3-yl)-phenyl]-3-(4-trifluoromethyl-phenyl)-urea |
| 138 | 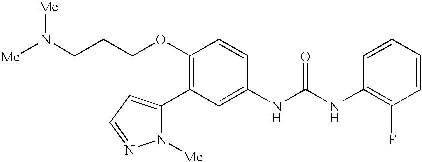 | 1-[4-(3-Dimethylamino-propoxy)-3-(2-methyl-2H-pyrazol-3-yl)-phenyl]-3-(2-fluoro-phenyl)-urea |
| 139 | 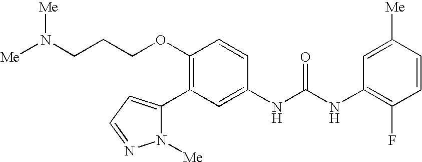 | 1-[4-(3-Dimethylamino-propoxy)-3-(2-methyl-2H-pyrazol-3-yl)-phenyl]-3-(2-fluoro-5-methyl-phenyl)-urea |
| 140 | 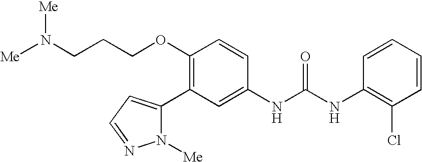 | 1-(2-Chloro-phenyl)-3-[4-(3-dimethylamino-propoxy)-3-(2-methyl-2H-pyrazol-3-yl)-phenyl]-urea |
| 141 | 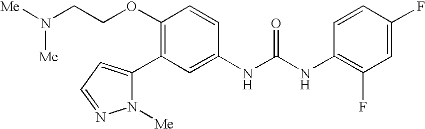 | 1-(2,4-Difluoro-phenyl)-3-[4-(2-dimethylamino-ethoxy)-3-(2-methyl-2H-pyrazol-3-yl)-phenyl]-urea |
| 142 | 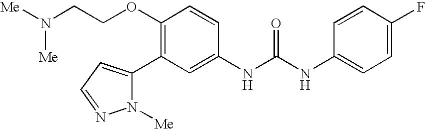 | 1-[4-(2-Dimethylamino-ethoxy)-3-(2-methyl-2H-pyrazol-3-yl)-phenyl]-3-(4-fluoro-phenyl)-urea |
| 143 | 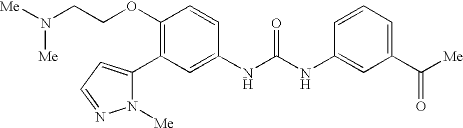 | 1-(3-Acetyl-phenyl)-3-[4-(2-dimethylamino-ethoxy)-3-(2-methyl-2H-pyrazol-3-yl)-phenyl]-urea |
| 144 | 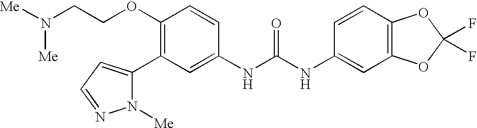 | 1-(2,2-Difluoro-benzo[1,3]dioxol-5-yl)-3-[4-(2-dimethylamino-ethoxy)-3-(2-methyl-2H-pyrazol-3-yl)-phenyl]-urea |
| 145 | 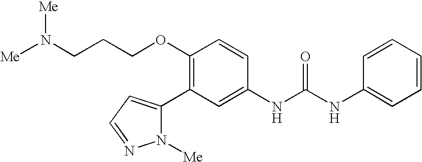 | 1-[4-(3-Dimethylamino-propoxy)-3-(2-methyl-2H-pyrazol-3-yl)-phenyl]-3-phenyl-urea |
| 146 | 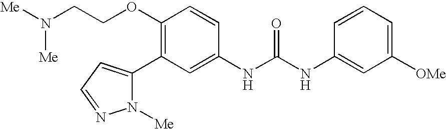 | 1-[4-(2-Dimethylamino-ethoxy)-3-(2-methyl-2H-pyrazol-3-yl)-phenyl]-3-(3-methoxy-phenyl)-urea |
| 147 | 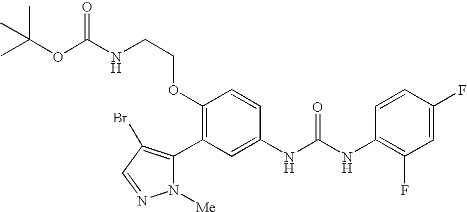 | (2-{2-(4-Bromo-2-methyl-2H-pyrazol-3-yl)-4-[3-(2,4-difluoro-phenyl)-ureido]-phenoxy}-ethyl)-carbamic acid tert-butyl ester |
| 148 | 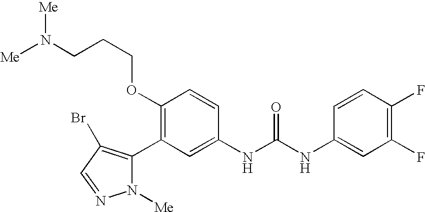 | 1-[3-(4-Bromo-2-methyl-2H-pyrazol-3-yl)-4-(3-dimethylamino-propoxy)-phenyl]-3-(3,4-difluoro-phenyl)-urea |
| 149 | 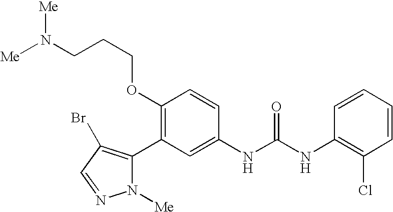 | 1-[3-(4-Bromo-2-methyl-2H-pyrazol-3-yl)-4-(3-dimethylamino-propoxy)-phenyl]-3-(2-chloro-phenyl)-urea |
| 150 | 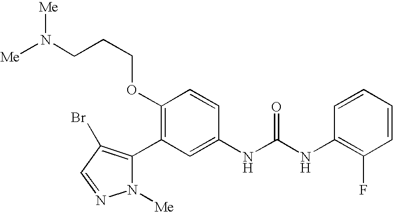 | 1-[3-(4-Bromo-2-methyl-2H-pyrazol-3-yl)-4-(3-dimethylamino-propoxy)-phenyl]-3-(2-fluoro-phenyl)-urea |
| 151 | 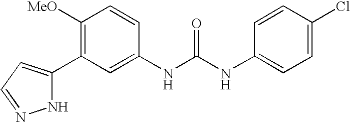 | 1-(4-Chloro-phenyl)-3-[4-methoxy-3-(2H-pyrazol-3-yl)-phenyl]-urea |
| 152 | 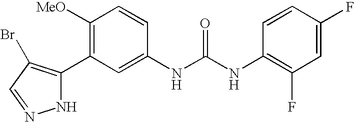 | 1-[3-(4-Bromo-2H-pyrazol-3-yl)-4-methoxy-phenyl]-3-(2,4-difluoro-phenyl)-urea |
| 153 | 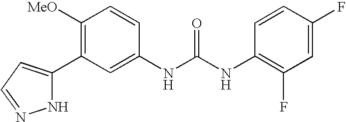 | 1-(2,4-Difluoro-phenyl)-3-[4-methoxy-3-(2H-pyrazol-3-yl)-phenyl]-urea |
| 154 | 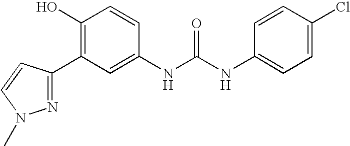 | 1-(4-Chloro-phenyl)-3-[4-hydroxy-3-(1-methyl-1H-pyrazol-3-yl)-phenyl]-urea |
| 155 | 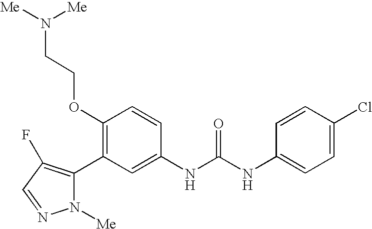 | 1-(4-Chloro-phenyl)-3-[4-(2-dimethylamino-ethoxy)-3-(4-fluoro-2-methyl-2H-pyrazol-3-yl)-phenyl]-urea |
| 156 | 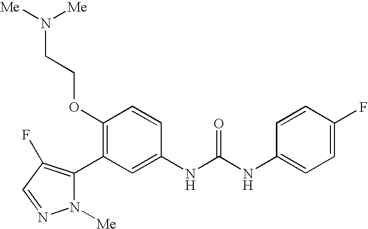 | 1-[4-(2-Dimethylamino-ethoxy)-3-(4-fluoro-2-methyl-2H-pyrazol-3-yl)-phenyl]-3-(4-fluoro-phenyl)-urea |
| 157 | 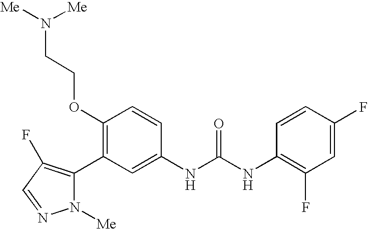 | 1-(2,4-Difluoro-phenyl)-3-[4-(2-dimethylamino-ethoxy)-3-(4-fluoro-2-methyl-2H-pyrazol-3-yl)-phenyl]-urea |
| 158 | 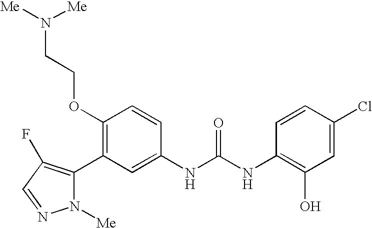 | 1-(4-Chloro-2-hydroxy-phenyl)-3-[4-(2-dimethylamino-ethoxy)-3-(4-fluoro-2-methyl-2H-pyrazol-3-yl)-phenyl]-urea |
| 159 | 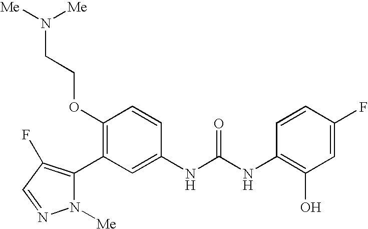 | 1-[4-(2-Dimethylamino-ethoxy)-3-(4-fluoro-2-methyl-2H-pyrazol-3-yl)-phenyl]-3-(4-fluoro-2-hydroxy-phenyl)-urea |
| 160 | 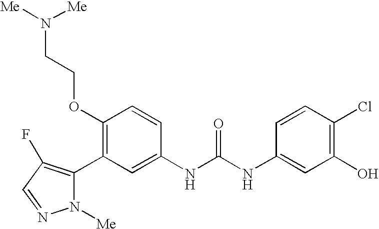 | 1-(4-Chloro-3-hydroxy-phenyl)-3-[4-(2-dimethylamino-ethoxy)-3-(4-fluoro-2-methyl-2H-pyrazol-3-yl)-phenyl]-urea |
| 161 | 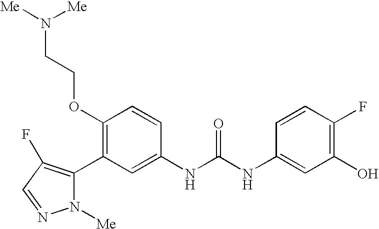 | 1-[4-(2-Dimethylamino-ethoxy)-3-(4-fluoro-2-methyl-2H-pyrazol-3-yl)-phenyl]-3-(4-fluoro-3-hydroxy-phenyl)-urea |
| 162 | 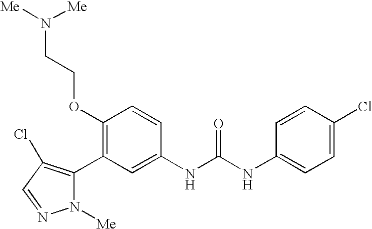 | 1-[3-(4-Chloro-2-methyl-2H-pyrazol-3-yl)-4-(2-dimethylamino-ethoxy)-phenyl]-3-(4-chloro-phenyl)-urea |
| 163 | 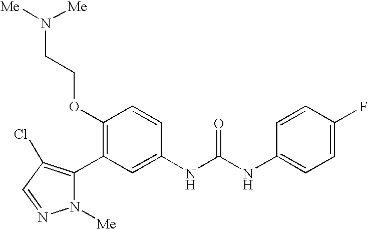 | 1-[3-(4-Chloro-2-methyl-2H-pyrazol-3-yl)-4-(2-dimethylamino-ethoxy)-phenyl]-3-(4-fluoro-phenyl)-urea |
| 164 | 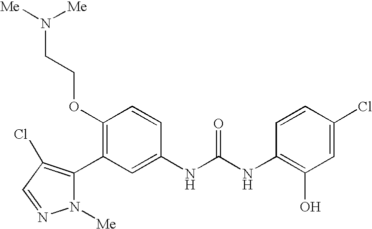 | 1-(4-Chloro-2-hydroxy-phenyl)-3-[3-(4-chloro-2-methyl-2H-pyrazol-3-yl)-4-(2-dimethylamino-ethoxy)-phenyl]-urea |
| 165 | 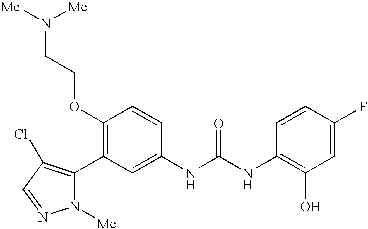 | 1-[3-(4-Chloro-2-methyl-2H-pyrazol-3-yl)-4-(2-dimethylamino-ethoxy)-phenyl]-3-(4-fluoro-2-hydroxy-phenyl)-urea |
| 166 | 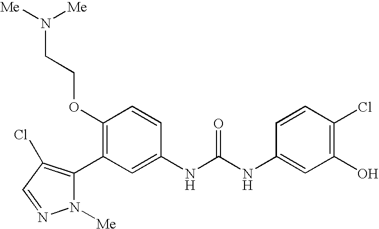 | 1-(4-Chloro-3-hydroxy-phenyl)-3-[3-(4-chloro-2-methyl-2H-pyrazol-3-yl)-4-(2-dimethylamino-ethoxy)-phenyl]-urea |
| 167 | 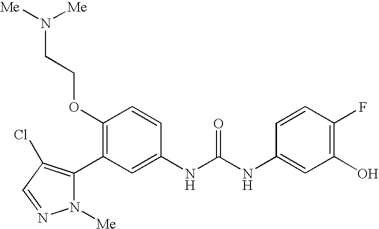 | 1-[3-(4-Chloro-2-methyl-2H-pyrazol-3-yl)-4-(2-dimethylamino-ethoxy)-phenyl]-3-(4-fluoro-3-hydroxy-phenyl)-urea |
| 168 | 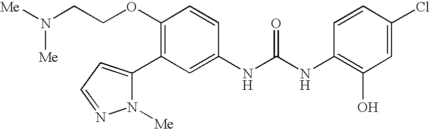 | 1-(4-Chloro-2-hydroxy-phenyl)-3-[4-(2-dimethylamino-ethoxy)-3-(2-methyl-2H-pyrazol-3-yl)-phenyl]-urea |
| 169 | 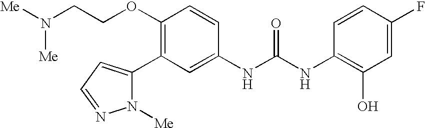 | 1-[4-(2-Dimethylamino-ethoxy)-3-(2-methyl-2H-pyrazol-3-yl)-phenyl]-3-(4-fluoro-2-hydroxy-phenyl)-urea |
| 170 | 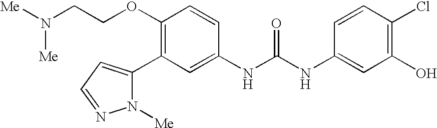 | 1-(4-Chloro-3-hydroxy-phenyl)-3-[4-(2-dimethylamino-ethoxy)-3-(2-methyl-2H-pyrazol-3-yl)-phenyl]-urea |
| 171 | 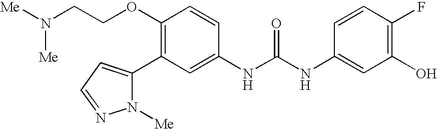 | 1-[4-(2-Dimethylamino-ethoxy)-3-(2-methyl-2H-pyrazol-3-yl)-phenyl]-3-(4-fluoro-3-hydroxy-phenyl)-urea |
| 172 | 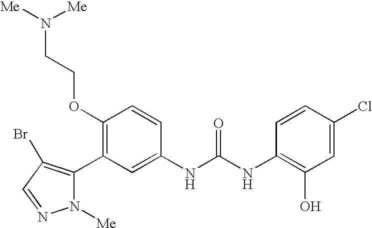 | 1-[3-(4-Bromo-2-methyl-2H-pyrazol-3-yl)-4-(2-dimethylamino-ethoxy)-phenyl]-3-(4-chloro-2-hydroxy-phenyl)-urea |
| 173 | 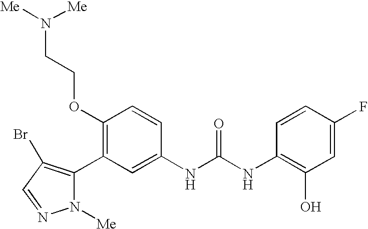 | 1-[3-(4-Bromo-2-methyl-2H-pyrazol-3-yl)-4-(2-dimethylamino-ethoxy)-phenyl]-3-(4-fluoro-2-hydroxy-phenyl)-urea |
| 174 | 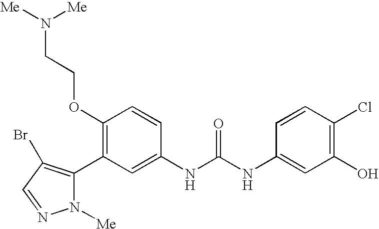 | 1-[3-(4-Bromo-2-methyl-2H-pyrazol-3-yl)-4-(2-dimethylamino-ethoxy)-phenyl]-3-(4-chloro-3-hydroxy-phenyl)-urea |
| 175 | 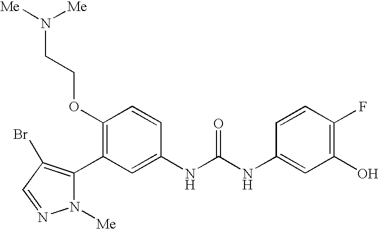 | 1-[3-(4-Bromo-2-methyl-2H-pyrazol-3-yl)-4-(2-dimethylamino-ethoxy)-phenyl]-3-(4-fluoro-3-hydroxy-phenyl)-urea |
| 176 | 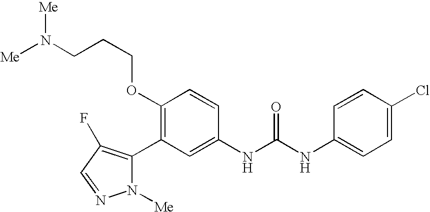 | 1-(4-Chloro-phenyl)-3-[4-(3-dimethylamino-propoxy)-3-(4-fluoro-2-methyl-2H-pyrazol-3-yl)-phenyl]-urea |
| 177 | 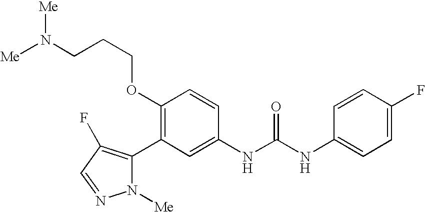 | 1-[4-(3-Dimethylamino-propoxy)-3-(4-fluoro-2-methyl-2H-pyrazol-3-yl)-phenyl]-3-(4-fluoro-phenyl)-urea |
| 178 | 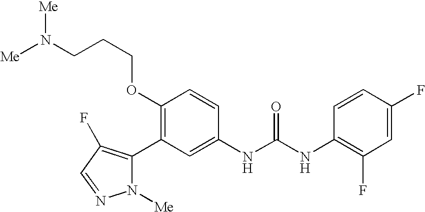 | 1-(2,4-Difluoro-phenyl)-3-[4-(3-dimethylamino-propoxy)-3-(4-fluoro-2-methyl-2H-pyrazol-3-yl)-phenyl]-urea |
| 179 | 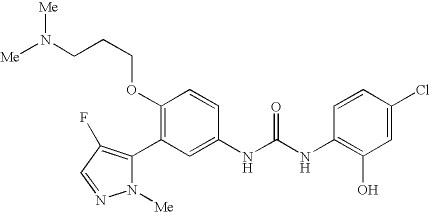 | 1-(4-Chloro-2-hydroxy-phenyl)-3-[4-(3-dimethylamino-propoxy)-3-(4-fluoro-2-methyl-2H-pyrazol-3-yl)-phenyl]-urea |
| 180 | 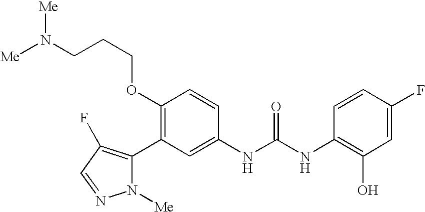 | 1-[4-(3-Dimethylamino-propoxy)-3-(4-fluoro-2-methyl-2H-pyrazol-3-yl)-phenyl]-3-(4-fluoro-2-hydroxy-phenyl)-urea |
| 181 | 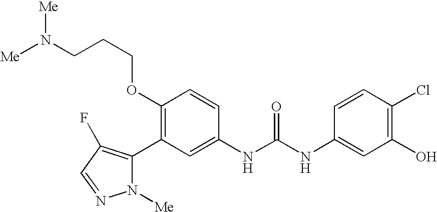 | 1-(4-Chloro-3-hydroxy-phenyl)-3-[4-(3-dimethylamino-propoxy)-3-(4-fluoro-2-methyl-2H-pyrazol-3-yl)-phenyl]-urea |
| 182 | 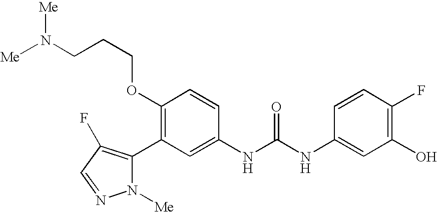 | 1-[4-(3-Dimethylamino-propoxy)-3-(4-fluoro-2-methyl-2H-pyrazol-3-yl)-phenyl]-3-(4-fluoro-3-hydroxy-phenyl)-urea |
| 183 | 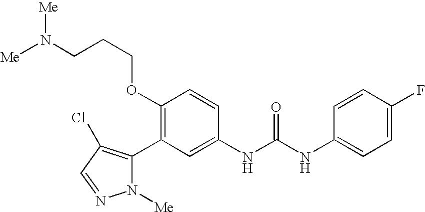 | 1-[3-(4-Chloro-2-methyl-2H-pyrazol-3-yl)-4-(3-dimethylamino-propoxy)-phenyl]-3-(4-fluoro-phenyl)-urea |
| 184 | 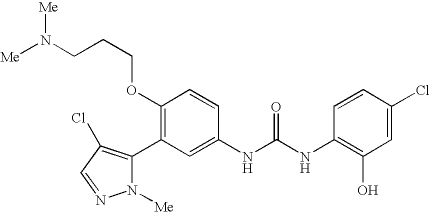 | 1-(4-Chloro-2-hydroxy-phenyl)-3-[3-(4-chloro-2-methyl-2H-pyrazol-3-yl)-4-(3-dimethylamino-propoxy)-phenyl]-urea |
| 185 | 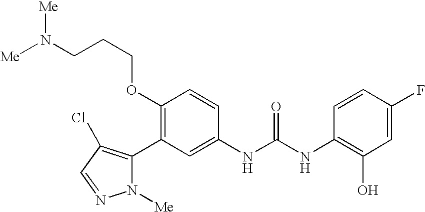 | 1-[3-(4-Chloro-2-methyl-2H-pyrazol-3-yl)-4-(3-dimethylamino-propoxy)-phenyl]-3-(4-fluoro-2-hydroxy-phenyl)-urea |
| 186 | 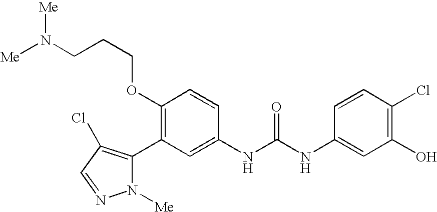 | 1-(4-Chloro-3-hydroxy-phenyl)-3-[3-(4-chloro-2-methyl-2H-pyrazol-3-yl)-4-(3-dimethylamino-propoxy)-phenyl]-urea |
| 187 | 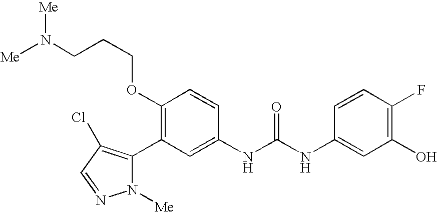 | 1-[3-(4-Chloro-2-methyl-2H-pyrazol-3-yl)-4-(3-dimethylamino-propoxy)-phenyl]-3-(4-fluoro-3-hydroxy-phenyl)-urea |
| 188 | 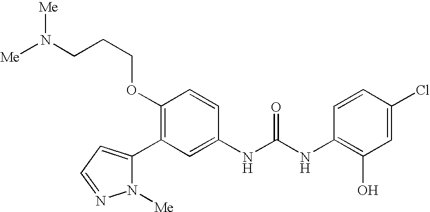 | 1-(4-Chloro-2-hydroxy-phenyl)-3-[4-(3-dimethylamino-propoxy)-3-(2-methyl-2H-pyrazol-3-yl)-phenyl]-urea |
| 189 | 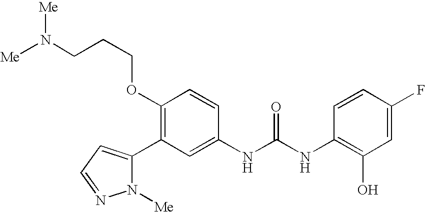 | 1-[4-(3-Dimethylamino-propoxy)-3-(2-methyl-2H-pyrazol-3-yl)-phenyl]-3-(4-fluoro-2-hydroxy-phenyl)-urea |
| 190 | 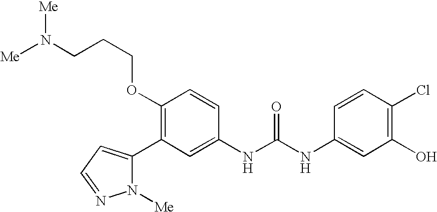 | 1-(4-Chloro-3-hydroxy-phenyl)-3-[4-(3-dimethylamino-propoxy)-3-(2-methyl-2H-pyrazol-3-yl)-phenyl]-urea |
| 191 | 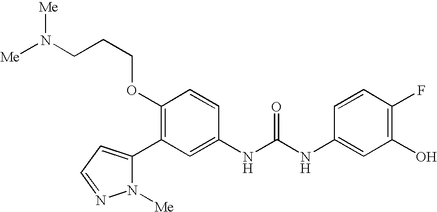 | 1-[4-(3-Dimethylamino-propoxy)-3-(2-methyl-2H-pyrazol-3-yl)-phenyl]-3-(4-fluoro-3-hydroxy-phenyl)-urea |
| 192 | 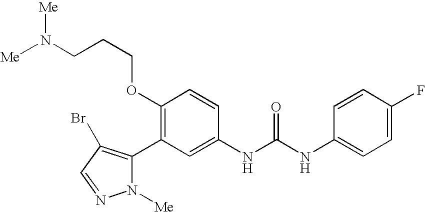 | 1-[3-(4-Bromo-2-methyl-2H-pyrazol-3-yl)-4-(3-dimethylamino-propoxy)-phenyl]-3-(4-fluoro-phenyl)-urea |
| 193 | 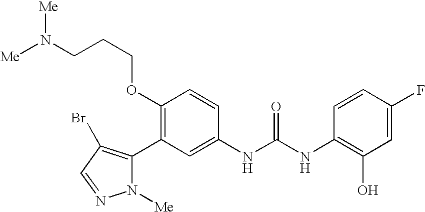 | 1-[3-(4-Bromo-2-methyl-2H-pyrazol-3-yl)-4-(3-dimethylamino-propoxy)-phenyl]-3-(4-chloro-2-hydroxy-phenyl)-urea |
| 194 | 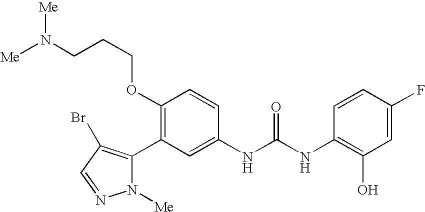 | 1-[3-(4-Bromo-2-methyl-2H-pyrazol-3-yl)-4-(3-dimethylamino-propoxy)-phenyl]-3-(4-fluoro-2-hydroxy-phenyl)-urea |
| 195 | 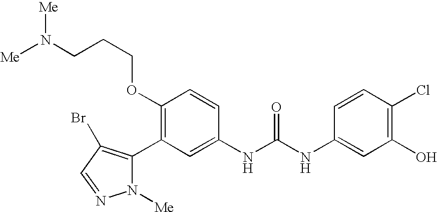 | 1-[3-(4-Bromo-2-methyl-2H-pyrazol-3-yl)-4-(3-dimethylamino-propoxy)-phenyl]-3-(4-chloro-3-hydroxy-phenyl)-urea |
| 196 | 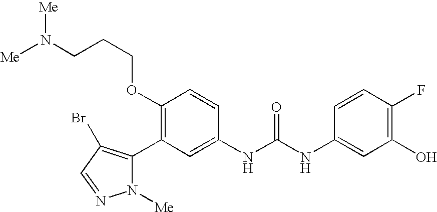 | 1-[3-(4-Bromo-2-methyl-2H-pyrazol-3-yl)-4-(3-dimethylamino-propoxy)-phenyl]-3-(4-fluoro-3-hydroxy-phenyl)-urea |

Additionally, compounds of the present invention, such as Formula (I) and related Formulae, encompass all pharmaceutically acceptable salts, solvates, and particularly hydrates, thereof.

The compounds of the present invention may be prepared as described in International Application No. PCT/US2004/023488, the disclosure of which is herein incorporated by reference in its entirety.

The present invention also encompasses diastereomers as well as optical isomers, e.g. mixtures of enantiomers including racemic mixtures, as well as individual enantiomers and diastereomers, which arise as a consequence of structural asymmetry in certain compounds of the invention. Separation of the individual isomers or selective synthesis of the individual isomers is accomplished by application of various methods which are well known to practitioners in the art.

Constitutively Active Human 5HT_{2A}

For convenience, the sequence information regarding the non-endogenous, constitutively active human 5-HT2A and identifiers are set forth in TABLE 4:

TABLE 4

| IDENTIFIER | RECEPTOR | SEQ. ID. NO: | FIGS. |
|---|---|---|---|
| AP-3 cDNA | 5-HT_{2A} | 27 | 6a |
| AP-3 | 5-HT_{2A} | 28 | 6b |
| AP-4 cDNA | 5-HT_{2A} | 29 | 7a |
| AP-4 | 5-HT_{2A} | 30 | 7b |

Indications and Methods of Prophylaxis and/or Treatment

The compounds disclosed herein are useful for the prophylaxis or treatment of a disease, condition or disorder related to JC virus infection of an individual through 5-HT_{2A}. Compounds of the present invention having inverse agonist activity at 5-HT_{2A} are useful in meeting an unmet medical need for the prophylaxis or treatment of progressive multifocal encephalopathy.

### Representative Methods of the Invention:

One aspect of the present invention relates to a method of prophylaxis or treatment of progressive multifocal encephalopathy comprising administering to an individual in need thereof a therapeutically effective amount of a compound of the invention, wherein the compound is a diaryl or arylheteroaryl urea derivative according to Formula (I).

One aspect of the present invention relates to a method of prophylaxis or treatment of progressive multifocal encephalopathy comprising administering to an individual in need thereof a therapeutically effective amount of a pharmaceutical composition, wherein the pharmaceutical composition comprises a compound of the invention and a pharmaceutically acceptable carrier, wherein the compound is a diaryl or arylheteroaryl urea derivative according to Formula (I).

One aspect of the present invention relates to a method of using a compound of the invention for the preparation of a medicament for the prophylaxis or treatment of progressive multifocal encephalopathy, wherein the compound is a diaryl or arylheteroaryl urea derivative according to Formula (I).

In some embodiments, the individual in need of prophylaxis or treatment has a lymphoproliferative disorder. In some embodiments, the lymphoproliferative disorder is leukemia or lymphoma. In some embodiments, the leukemia or lymphoma is chronic lymphocytic leukemia, Hodgkin's disease, or the like.

In some embodiments, the individual in need of prophylaxis or treatment has a myeloproliferative disorder.

In some embodiments, the individual in need of prophylaxis or treatment has carcinomatosis.

In some embodiments, the individual in need of prophylaxis or treatment has a granulomatous or inflammatory disease. In some embodiments, the granulomatous or inflammatory disease is tuberculosis or sarcoidosis.

In some embodiments, the individual in need of prophylaxis or treatment is immunocompromised. In some embodiments, the immunocompromised individual has impaired cellular immunity. In some embodiments, the impaired cellular immunity comprises impaired T-cell immunity.

In some embodiments, the individual in need of prophylaxis or treatment is infected with HIV. In some embodiments, the HIV-infected individual has a CD4+ cell count of ≦200/mm³. In some embodiments, the HIV-infected individual has AIDS. In some embodiments, the HIV-infected individual has AIDS-related complex (ARC). In certain embodiments, ARC is defined as the presence of two successive CD4+ cell counts below 200/mm³ and at least two of the following signs or symptoms: oral hairy leukoplakia, recurrent oral candidiasis, weight loss of at least 2.5 kg or 10% of body weight within last six months, multidermatomal herpes zoster, temperature above 38.5° C. for more than 14 consecutive days or more than 15 days in a 30-day period, or diarrhea with more than three liquid stools per day for at least 30 days [see, e.g., Yamada et al., Clin. Diagn. Virol. (1993) 1:245-256].

In some embodiments, the individual in need of prophylaxis or treatment is undergoing immunosuppressive therapy. In some embodiments, the immunosuppressive therapy comprises administering an immunosuppressive agent [see, e.g., Mueller, Ann Thorac Surg (2004) 77:354-362; and Krieger and Emre, Pediatr Transplantation (2004) 8:594-599]. In some embodiments, the immunosuppressive therapy comprises administering an immunosuppressive agent selected from the group consisting of: corticosteroids (for example, prednisone and the like), calcineurin inhibitors (for example, cyclosporine, tacrolimus, and the like), antiproliferative agents (for example, azathioprine, mycophenolate mofetil, sirolimus, everolimus, and the like), T-cell depleting agents (for example, OKT®3 monoclonal antibody (mAb), anti-CD3 immunotoxin FN18-CRM9, Campath-1H (anti-CD52) mAb, anti-CD4 mAb, anti-T cell receptor mAb, and the like), anti-IL-2 receptor (CD25) mAb (for example, basiliximab, daclizumab, and the like), inhibitors of co-stimulation (for example, CTLA4-Ig, anti-CD154 (CD40 ligand) mAb, and the like), deoxyspergualin and analogs thereof (for example, 15-DSG, LF-08-0299, LF14-0195, and the like), leflunomide and analogs thereof (for example, leflunomide, FK778, FK779, and the like), FTY720, and anti-CD45 RB monoclonal antibody. In some embodiments, the immunosuppressive agent and said compound or pharmaceutical composition are administered in separate dosage forms. In some embodiments, the immunosuppressive agent and said compound or pharmaceutical composition are administered in a single dosage form.

In some embodiments, the individual in need of prophylaxis or treatment is undergoing immunosuppressive therapy after organ transplantation. In some embodiments, the organ is liver, kidney, lung, heart, or the like [see, e.g., Singh et al., Transplantation (2000) 69:467-472].

In some embodiments, the individual in need of prophylaxis or treatment is undergoing treatment for a rheumatic disease. In some embodiments, the rheumatic disease is systemic lupus erythematosus or the like.

In some embodiments, the compound of the invention is a 5-HT_{2A} ligand. In some embodiments, the compound of the invention is a selective 5-HT_{2A} ligand.

In some embodiments, the compound of the invention inhibits JC virus infection of human glial cells.

In some embodiments, the compound of the invention is a 5-HT_{2A} inverse agonist. In some embodiments, the compound of the invention is a selective 5-HT_{2A} inverse agonist.

In some embodiments, the compound of the invention crosses the blood-brain barrier.

In some embodiments, the individual is a human.

### Pharmaceutical Compositions

A further aspect of the present invention pertains to pharmaceutical compositions comprising one or more compounds as described herein and one or more pharmaceutically acceptable carriers. Some embodiments pertain to pharmaceutical compositions comprising a compound of the present invention and a pharmaceutically acceptable carrier.

Some embodiments of the present invention include a method of producing a pharmaceutical composition comprising admixing at least one compound according to any of the compound embodiments disclosed herein and a pharmaceutically acceptable carrier.

Formulations may be prepared by any suitable method, typically by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tabletting lubricants, and disintegrants may be used in tablets and capsules for oral administration. Liquid preparations for oral administration may be in the form of solutions, emulsions, aqueous or oily suspensions, and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives, and flavorings and colorants may be added to the liquid preparations. Parenteral dosage forms may be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro, A. R., et al.).

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

The invention thus further provides pharmaceutical formulations comprising a compound of the invention or a pharmaceutically acceptable salt or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate or physiologically functional derivative thereof can be used as active ingredients in pharmaceutical compositions, specifically as 5-HT_{2A} receptor modulators. By the term “active ingredient” is defined in the context of a “pharmaceutical composition” and shall mean a component of a pharmaceutical composition that provides the primary pharmacological effect, as opposed to an “inactive ingredient” which would generally be recognized as providing no pharmaceutical benefit.

The dose when using the compounds of the present invention can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg, and about 0.001 mg to about 25 mg. Multiple doses may be administered during the day, especially when relatively large amounts are deemed to be needed, for example 2, 3 or 4, doses. Depending on the individual and as deemed appropriate from the patient's physician or care-giver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate iii vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogeneous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well-known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed in Journal of Pharmaceutical Science, 66, 2 (1977); incorporated herein by reference in its entirety.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, “Pro-drugs as Novel Delivery Systems,” Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier. It will be understood that the scope of the combination-therapy of the compounds of the present invention with other pharmaceutical agents is not limited to those listed herein, supra or infra, but includes in principle any combination with any pharmaceutical agent or pharmaceutical composition useful for the treatment of the diseases, conditions or disorders of the present invention in an individual.

Suitable pharmaceutical agents that can be used in conjunction with compounds of the present invention include antiretrovirals [see, e.g., Turpin, Expert Rev Anti Infect Ther (2003) 1:97-128]. Some embodiments of the present invention include methods of treatment of a disease, disorder or condition as described herein comprising administering to an individual in need of such treatment a therapeutically effective amount or dose of a compound of the present invention in combination with at least one pharmaceutical agent selected from the group consisting of: nucleoside reverse transcriptase inhibitors (for example, Retrovir®, Epivir®, Combivir®, Hivid®, Videx®, Trizvir®, Zerit®, Ziagen®, Vired®, Emtricitabine, DAPD, and the like), non-nucleoside reverse transcriptase inhibitors (for example, Virammune®, Rescriptor®, Sustiva®, GW687, DPC083, TMC 125, Emivirine, Capravirine, BMS 561390, UC-781 and other oxathiin carboxyanilides, SJ-3366, Alkenyldiarylmethane (ADAM), Tivirapine, Calanolide A, HBY097, Loviride, HEPT Family Derivatives, TIBO Derivatives, and the like), protease inhibitors (for example, Fortovase®, Invirase®, Novir®, Crixivan®, Viracep®, Ageberase®, Kaletra®, Atazanavir, Tipranavir, DMP450, and the like), inhibitors of HIV-cell interaction (for example, soluble CD4, toxin-conjugated CD4, monoclonal antibodies to CD4 or gp120, PRO 542, dextran sulfate, Rersobene, FP-23199, Cyanovirin-N, Zintevir (T30177, AR177), L-chicoric acid and derivatives, and the like), coreceptor inhibitors ligands (for example, R5, X4, modified ligands (R5), modified ligands (X4), and the like), coreceptor inhibitors X4 (for example, T22, T134, ALX40-4C, AMD3100, bycyclam derivatives, and the like), coreceptor inhibitors R5 (for example, TAK-779, SCH-C (SCH-351125), SCH-D (SCH-350634), NSC 651016, ONO Pharmaceutical, Merck, and the like), fusion inhibitors (for example, Fuzeon® (T-20, DP 178, enfuvritide) trimeris, T-1249, TMC125, and the like), integrase inhibitors (for example, 5CITEP, L731,988, L708,906, L-870,812, S-1360, and the like), NCp7 nucleocapsid Zn finger inhibitors (for example, NOBA, DIBA, dithianes, PD-161374, pyridinioalkanoyl thioesters (PATES), azodicarbonamide (ADA), cyclic 2,2 dithio bisbenzamide, and the like), RNase H inhibitors (for example, BBHN, CPHM PD-26388, and the like), Tat inhibitors (for example, dominant negative mutants, Ro24-7429, Ro5-3335, and the like), Rev inhibitors (for example, dominant negative mutants, Leptomycin B, PKF050-638, and the like), transcriptional inhibitors (for example, Temacrazine, K-12 and K-37, EM2487, and the like), inhibitors of HIV assembly/maturation (for example, CAP-1 and CAP-2, and the like), and pharmaceutical agents directed to cellular anti-HIV targets (for example, LB6-B275 and HRM1275, Cdk9 inhibitors, and the like).

In a certain embodiment, a compound of the invention can be used in conjunction with highly active antiretroviral therapy (HAART). When antiretroviral drugs are used in combinations of three or four drugs, this treatment is called HAART [see, e.g., Portegies, et al., Eur. J. Neurol. (2004) 11:297-304].

It is noted that when the 5-HT_{2A} receptor modulators are utilized as active ingredients in a pharmaceutical composition, these are not intended for use only in humans, but in other non-human mammals as well. Indeed, recent advances in the area of animal health-care mandate that consideration be given for the use of active agents, such as 5-HT_{2A} receptor modulators, for the treatment of a 5-HT_{2A} mediated disease or disorder in domestic animals (e.g., cats and dogs) and in other domestic animals (e.g., such as cows, chickens, fish, etc.). Those of ordinary skill in the art are readily credited with understanding the utility of such compounds in such settings.

### Other Utilities

Another object of the present invention relates to radio-labeled compounds of the present invention that would be useful in assays, both in vitro and in vivo, for localizing and quantitating the 5-HT_{2A} receptor in tissue samples, including human, and for identifying 5-HT_{2A} receptor ligands by inhibition binding of a radio-labeled compound. It is a further object of this invention to develop novel 5-HT_{2A} receptor assays of which comprise such radio-labeled compounds.

The present invention embraces isotopically-labeled compounds of the present invention. An “isotopically” or “radio-labeled” compounds are those which are identical to compounds disclosed herein, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵ Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro 5-HT_{2A} receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound of Formula (I) that has incorporated at least one radionuclide; in some embodiments the radionuclide is selected from the group consisting of ³H, ¹⁴C, ¹²⁵I, ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Schemes supra and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the more scarce radio-isotope or nonradio-active isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)4] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

A radio-labeled 5-HT_{2A} receptor compound of Formula (I) can be used in a screening assay to identify/evaluate compounds. In general terms, a newly synthesized or identified compound (i.e., test compound) can be evaluated for its ability to reduce binding of the “radio-labeled compound of Formula (I)” to the 5-HT_{2A} receptor. Accordingly, the ability of a test compound to compete with the “radio-labeled compound of Formula (I)” for the binding to the 5-HT_{2A} receptor directly correlates to its binding affinity.

The labeled compounds of the present invention bind to the 5-HT_{2A} receptor. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM, and in still yet another embodiment the labeled inhibitor has an IC₅₀ less than about 0.1 μM.

Other uses of the disclosed receptors and methods will become apparent to those in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLES

### Example 1

Receptor cDNA
A. Construction of Constitutively Active 5-HT_{2C} Receptor cDNA

1. Endogenous Human 5-HT_{2C}

The cDNA encoding endogenous human 5-HT_{2C} receptor was obtained from human brain poly-A⁺ RNA by RT-PCR. The 5′ and 3′ primers were derived from the 5′ and 3′ untranslated regions and contained the following sequences:

| | |
|---|---|
| 5′-GACCTCGAGGTTGCTTAAGACTGAAGCA-3′ | (SEQ.ID.NO.: 1) |
| 5′-ATTTCTAGACATATGTAGCTTGTACCGT-3′ | (SEQ.ID.NO.: 2) |

PCR was performed using either TaqPlus™ precision polymerase (Stratagene) or rTth™ polymerase (Perkin Elmer) with the buffer systems provided by the manufacturers, 0.25 μM of each primer, and 0.2 mM of each of the four (4) nucleotides. The cycle condition was 30 cycles of 94° C. for 1 minute, 57° C. for 1 minute and 72° C. for 2 minutes. The 1.5 kb PCR fragment was digested with Xho I and Xba I and subcloned into the Sal I-Xba I site of pBluescript.

The derived cDNA clones were fully sequenced and found to correspond to published sequences.

2. AP-1 cDNA

The cDNA containing a S310K mutation (AP-1 cDNA) in the third intracellular loop of the human 5-HT_{2C} receptor was constructed by replacing the Sty I restriction fragment containing amino acid 310 with synthetic double stranded oligonucleotides encoding the desired mutation. The sense strand sequence utilized had the following sequence:

(SEQ.ID.NO: 3)

5′-CTAGGGGCACCATGCAGGCTATCAACAATGAAAGAAAAGCTAAGAAA
GTC-3′

and the antisense strand sequence utilized had the following sequence:

(SEQ. ID. NO: 4)

5′-CAAGGACTTTCTTAGCTTTTCTTTCATTGTTGATAGCCTGCATGGTG
CCC-3′.

B. Construction of Constitutively Active 5-HT_{2A} Receptor cDNA

1. Human 5-HT_{2A} (C322K; AP-2)

The cDNA containing the point mutation C322K in the third intracellular loop was constructed by using the Sph I restriction enzyme site, which encompasses amino acid 322. For the PCR procedure, a primer containing the C322K mutation:

(SEQ.ID.NO: 5)

5′-CAAAGAAAGTACTGGGCATCGTCTTCTTCCT-3′

was used along with the primer from the 3′ untranslated region SEQ.ID.NO:6.

(SEQ.ID.NO: 6)

5′-TGCTCTAGATTCCAGATAGGTGAAAA CTTG-3′

The resulting PCR fragment was then used to replace the 3′ end of the wild type 5-HT_{2A} cDNA by the T4 polymerase blunted Sph I site. PCR was performed using pfu polymerase (Stratagene) with the buffer system provided by the manufacturer and 10% DMSO, 0.25 mM of each primer, 0.5 mM of each of the 4 nucleotides. The cycle conditions were 25 cycles of 94° C. for 1 minute, 60° C. for 1 minute, and 72° C. for 1 minute.

2. AP-3 cDNA

The human 5-HT_{2A} cDNA with intracellular loop 3 (IC3) or IC3 and cytoplasmic tail replaced by the corresponding human 5-HT_{2C} cDNA was constructed using PCR-based mutagenesis.

(a) Replacement of IC3 Loop

The IC3 loop of human 5-HT_{2A} cDNA was first replaced with the corresponding human 5-HT_{2C} cDNA. Two separate PCR procedures were performed to generate the two fragments, Fragment A and Fragment B, that fuse the 5-HT_{2C} IC3 loop to the transmembrane 6 (TM6) of 5-HT_{2A}. The 237 bp PCR fragment, Fragment A, containing 5-HT_{2C} IC3 and the initial 13 bp of 5-HT_{2A} TM6 was amplified by using the following primers:

(SEQ.ID.NO: 7)

5′-CCGCTCGAGTACTGCGCCGACAAGCTTTGAT-3′

(SEQ.ID.NO: 8)

5′-CGATGCCCAGCACTTTCGAAGCTTTTCTTTCATTGTTG-3′

The template used was human 5-HT_{2C} cDNA.

The 529 bp PCR fragment, Fragment B, containing the C-terminal 13 bp of IC3 from 5-HT_{2C} and the C-terminal of 5-HT_{2A} starting at beginning of TM6, was amplified by using the following primers:

(SEQ.ID.NO: 9)

5′-AAAAGCTTCGAAAGTGCTGGGCATCGTCTTCTTCCT-3′

(SEQ.ID.NO: 10)

5′-TGCTCTAGATTCCAGATAGGTGAAAACTTG-3′

The template used was human 5-HT_{2A} cDNA.

Second round PCR was performed using Fragment A and Fragment B as co-templates with SEQ.ID.NO:7 and SEQ.ID.NO:10 (it is noted that the sequences for SEQ.ID.NOS.: 6 and 10 are the same) as primers. The resulting 740 bp PCR fragment, Fragment C, contained the IC3 loop of human 5-HT_{2C} fused to TM6 through the end of the cytoplasmic tail of human 5-HT_{2A}. PCR was performed using pfu™ polymerase (Stratagene) with the buffer system provided by the manufacturer, and 10% DMSO, 0.25 mM of each primer, and 0.5 mM of each of the four (4) nucleotides. The cycle conditions were 25 cycles of 94° C. for 1 minute, 57° C. (1st round PCR) or 60° C. (2nd round PCR) for 1 minute, and 72° C. for 1 minute (1st round PCR) or 90 seconds (2nd round PCR).

To generate a PCR fragment containing a fusion junction between the human 5-HT_{2A} TM5 and the IC3 loop of 5-HT_{2C}, four (4) primers were used. The two external primers, derived from human 5-HT_{2A}, had the following sequences:

| | |
|---|---|
| 5′-CGTGTCTCTCCTTACTTCA-3′ | (SEQ.ID.NO.: 11) |

The other primer used was SEQ.ID.NO.:6 (see note above regarding SEQ.ID.NOS. 6 and 11). The first internal primer utilized was an antisense strand containing the initial 13 bp of IC3 of 5-HT_{2C} followed by the terminal 23 bp derived from TM5 of 5-HT_{2A}:

(SEQ.ID.NO.: 12)

5′-TCGGCGCAGTACTTTGATAGTTAGAAAGTAGGTGAT-3′

The second internal primer was a sense strand containing the terminal 14 bp derived from TM5 of 5-HT_{2A} followed by the initial 24 bp derived from IC3 of 5-HT_{2C}:

(SEQ.ID.NO.: 13)

5′-TTCTAACTATCAAAGTACTGCGCCGACAAGCTTTGATG-3′.

PCR was performed using endogenous human 5-HT_{2A} and a co-template, Fragment C, in a 50 mL reaction volume containing 1× pfu buffer, 10% DMSO, 0.5 mM of each of the four (4) nucleotides, 0.25 mM of each external primer (SEQ.ID.NOS. 10 and 11), 0.06 mM of each internal primer (SEQ.ID.NOS. 12 and 13) and 1.9 units of pfu polymerase (Stratagene). The cycle conditions were 25 cycles of 94° C. for 1 minute, 52° C. for 1 minute, and 72° C. for 2 minutes and 10 seconds. The 1.3 kb PCR product was then gel purified and digested with Pst I and EcoR I. The resulting 1 kb Pst I-EcoR I fragment was used to replace the corresponding fragment in the endogenous human 5-HT_{2A} sequence to generate the mutant 5-HT_{2A} sequence encoding the IC3 loop of 5-HT2C.

(b) Replacement of the Cytoplasmic Tail

To replace the cytoplasmic tail of 5-HT_{2A} with that of 5-HT_{2C}, PCR was performed using a sense primer containing the C-terminal 22 bp of TM7 of endogenous human 5-HT_{2A} followed by the initial 21 bp of the cytoplasmic tail of endogenous human 5-HT_{2C}:

(SEQ.ID.NO: 14)

5′-TTCAGCAGTCAACCCACTAGTCTATACTCTGTTCAACAAAATT-3′

The antisense primer was derived from the 3′ untranslated region of endogenous human 5-HT_{2C}:

(SEQ.ID.NO: 15)

5′-ATTTCTAGACATATGTAGCTTGTACCGT-3′.

The resulting PCR fragment, Fragment D, contained the last 22 bp of endogenous human 5-HT_{2A} TM7 fused to the cytoplasmic tail of endogenous human 5-HT_{2C}. Second round PCR was performed using Fragment D and the co-template was endogenous human 5-HT_{2A} that was previously digested with Acc I to avoid undesired amplification. The antisense primer used was SEQ.ID.NO:15 (the sequences for SEQ.ID.NOS. 15 and 2 are the same) and the sense primer used was derived from endogenous human 5-HT_{2A}:

| | |
|---|---|
| 5′-ATCACCTACTTTCTAACTA-3′. | (SEQ,ID.NO: 16) |

PCR conditions were as set forth in Example 1 section B2(a) for the first round PCR, except that the annealing temperature was 48° C. and the extension time was 90 seconds. The resulting 710 bp PCR product was digested with Apa I and Xba I and used to replace the corresponding Apa I-Xba I fragment of either (a) endogenous human 5-HT_{2A}, or (b) 5-HT_{2A} with 2C IC₃ to generate (a) endogenous human 5-HT_{2A} with endogenous human 5-HT_{2C} cytoplasmic tail and (b) AP-3, respectively.

4. AP-4 cDNA

This mutant was created by replacement of the region of endogenous human 5-HT_{2A} from amino acid 247, the middle of TM5 right after Pro²⁴⁶, to amino acid 337, the middle of TM6 just before Pro³³⁸, by the corresponding region of AP-1 cDNA. For convenience, the junction in TM5 is referred to as the “2A-2C junction,” and the junction in TM6 is referred to as the “2C-2A junction.”

Three PCR fragments containing the desired hybrid junctions were generated. The 5′ fragment of 561 bp containing the 2A-2C junction in TM5 was generated by PCR using endogenous human 5-HT_{2A} as template, SEQ.ID.NO.:11 as the sense primer, and the antisense primer was derived from 13 bp of 5-HT_{2C} followed by 20 bp of 5-HT_{2A} sequence:

(SEQ.ID.NO: 17)

5′-CCATAATCGTCAGGGGAATGAAAAATGACACAA-3′

The middle fragment of the 323 bp contains endogenous human 5-HT_{2C} sequence derived from the middle of TM5 to the middle of TM6, flanked by 13 bp of 5-HT_{2A} sequences from the 2A-2C junction and the 2C-2A junction. This middle fragment was generated by using AP-1 cDNA as a template, a sense primer containing 13 bp of 5-HT_{2A} followed by 20 bp of 5-HT_{2C} sequences across the 2A-2C junction and having the sequence:

(SEQ.ID.NO: 18)

5′-ATTTTTCATTCCCCTGAGGATTATGGTGATTAC-3′;

and an antisense primer containing 13 bp of 5-HT_{2A} followed by 20 bp of 5-HT_{2C} sequences across the 2C-2A junction and having the sequence:

(SEQ.ID.NO: 19)

5′-TGATGAAGAAAGGGCACCACATGATCAGAAACA-3′.

The 3′ fragment of 487 bp containing the 2C-2A junction was generated by PCR using endogenous human 5-HT_{2A} as a template and a sense primer having the following sequence from the 2C-2A junction:

(SEQ.ID.NO: 20)

5′-GATCATGTGGTGCCCTTTCTTCATCACAAACAT-3′

and the antisense primer was SEQ.ID.NO:6 (see note above regarding SEQ.ID.NOS. 6 and 10).

Two second round PCR reactions were performed separately to link the 5′ and middle fragment (5′M PCR) and the middle and 3′ fragment (M3′ PCR). The 5′M PCR co-template used was the 5′ and middle PCR fragment as described above, the sense primer was SEQ.ID.NO:11 and the antisense primer was SEQ.ID.NO.:19. The 5′M PCR procedure resulted in an 857 bp PCR fragment.

The M3′ PCR used the middle and M3′ PCR fragment described above as the co-template, SEQ.ID.NO.: 18 as the sense primer and SEQ.ID.NO.:6 (see note above regarding SEQ.ID.NOS. 6 and 10) as the antisense primer, and generated a 784 bp amplification product. The final round of PCR was performed using the 857 bp and 784 bp fragments from the second round PCR as the co-template, and SEQ.ID.NO:11 and SEQ.ID.NO: 6 (see note above regarding SEQ.ID.NOS. 6 and 10) as the sense and the antisense primer, respectively. The 1.32 kb amplification product from the final round of PCR was digested with Pst I and Eco RI. Then resulting 1 kb Pst I-Eco RI fragment was used to replace the corresponding fragment of the endogenous human 5-HT_{2A} to generate mutant 5-HT_{2A} with 5-HT_{2C}: S310K/IC3. The Apa I-Xba fragment of AP-3 was used to replace the corresponding fragment in mutant 5-HT_{2A} with 5-HT_{2C}: S310K/IC3 to generate AP-4.

### Example 2

### Receptor Expression

A. pCMV

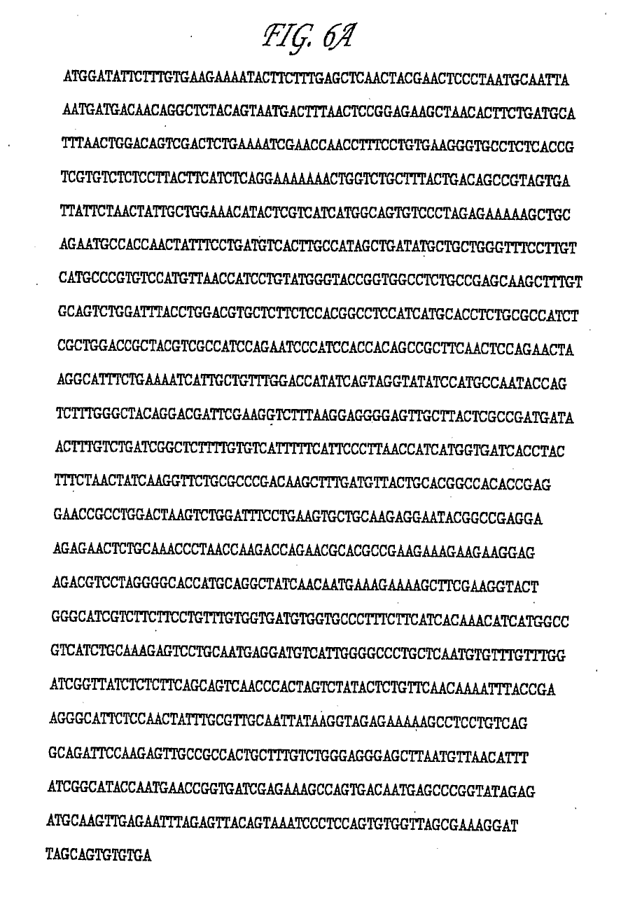

A variety of expression vectors are available to those in the art, for purposes of producing a polypeptide of interest in a cell. One suitable vector is pCMV, which is used in certain embodiments. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351. See FIG. 8. Other suitable expression vectors will be readily apparent to those of ordinary skill in the art.

B. Transfection Procedure

For the generic assay ([³⁵S]GTPγS; Example 3) and the antagonist binding assay (mesulergine; Example 3), transfection of COS-7 or 293T cells was accomplished using the following protocol.

On day one, 5×10⁶ COS-7 cells or 1×10⁷ 293T cells per 150 mm plate were plated out. On day two, two reaction tubes were prepared (the proportions to follow for each tube are per plate): tube A was prepared by mixing 20 μg DNA (e.g., pCMV vector; pCMV vector with receptor cDNA; etc.) in 1.2 ml serum free DMEM (Irvine Scientific, Ivine, Calif.); tube B was prepared by mixing 120 μl lipofectamine (Gibco BRL) in 1.2 ml serum free DMEM. Tubes A and B were then admixed by inversions (several times), followed by incubation at room temperature for 30-45 min. The admixture is referred to as the “transfection mixture”. Plated COS-7 cells were washed with 1× PBS, followed by addition of 10 ml serum free DMEM. 2.4 ml of the transfection mixture was then added to the cells, followed by incubation for 4 hrs at 37° C./5% CO₂. The transfection mixture was then removed by aspiration, followed by the addition of 25 ml of DMEM/10% Fetal Bovine Serum. Cells were then incubated at 37° C./5% CO₂. After 72 hr incubation, cells were then harvested and utilized for analysis.

### Example 3

### GTP Membrane Binding Scintillation Proximity Assay

The advantages of using [³⁵S]GTPγS binding to measure constitutive activation are that: (a) [³⁵S]GTPγS binding is generically applicable to all G protein-coupled receptors; and (b) [³⁵S]GTPγS binding is proximal at the membrane surface, thereby making it less likely to pick-up molecules which affect the intracellular cascade. The assay utilizes the ability of G protein-coupled receptors to stimulate [³⁵S]GTPγS binding to membranes expressing the relevant receptors. Therefore, the assay may be used to directly screen compounds at the disclosed serotonin receptors.

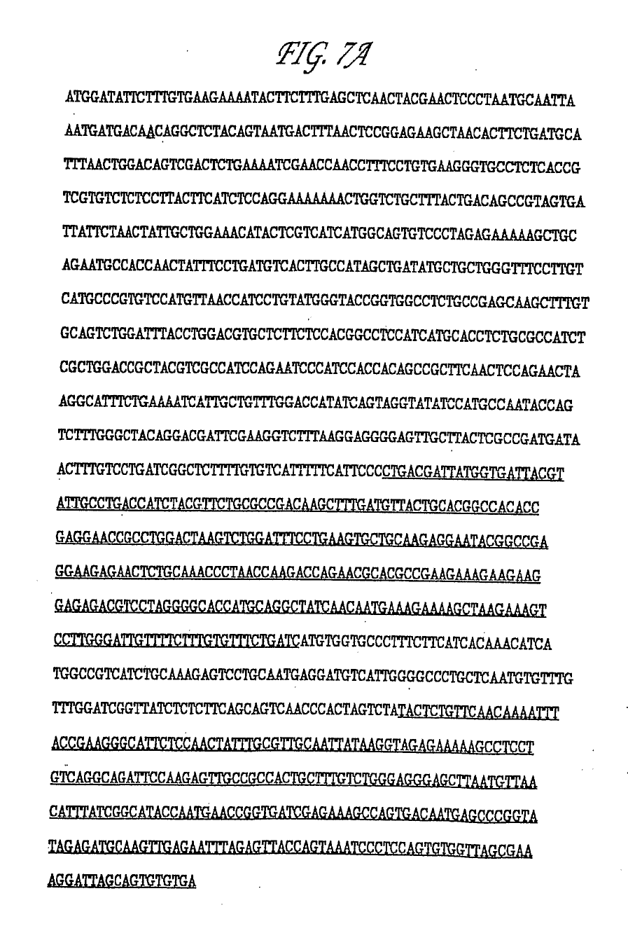

FIG. 9 demonstrates the utility of a scintillation proximity assay to monitor the binding of [³⁵S]GTPγS to membranes expressing, e.g., the endogenous human 5-HT_{2C} receptor expressed in COS cells. In brief, a preferred protocol for the assay is such that the assay was incubated in 20 mM HEPES, pH 7.4, binding buffer with 0.3 nM [³⁵S]GTPγS and 12.5 μg membrane protein and 1 μM GDP for 30 minutes. Wheatgerm agglutinin beads (25 μl; Amersham) were then added and the mixture was incubated for another 30 minutes at room temperature. The tubes were then centrifuged at 1500×g for 5 minutes at room temperature and then counted in a scintillation counter. As shown in FIG. 9, serotonin, which as the endogenous ligand activates the 5-HT_{2C} receptor, stimulated [³⁵S]GTPγS binding to the membranes in a concentration dependant manner. The stimulated binding was completely inhibited by 30 μM mianserin, a compound considered as a classical 5-HT_{2C} antagonist, but also known as a 5-HT_{2C} inverse agonist.

Although this assay measures agonist-induced binding of [³⁵S]GTPγS to membranes and can be routinely used to measure constitutive activity of receptors, the present cost of wheatgerm agglutinin beads may be prohibitive. A less costly but equally applicable alternative also meets the needs of large-scale screening. Flash plates and Wallac™ scintistrips may be used to format a high throughput [³⁵S]GTPγS binding assay. This technique allows one to monitor the tritiated ligand binding to the receptor while simultaneously monitoring the efficacy via [³⁵S]GTPγS binding. This is possible because the Wallac™ beta counter can switch energy windows to analyze both tritium and ³⁵S-labeled probes.

Also, this assay may be used for detecting of other types of membrane activation events that result in receptor activation. For example, the assay may be used to monitor ³²P phosphorylation of a variety of receptors (including G protein-coupled and tyrosine kinase receptors). When the membranes are centrifuged to the bottom of the well, the bound [³⁵S]GTPγS or the ³²P-phosphorylated receptor will activate the scintillant coated on the wells. Use of Scinti® strips (Wallac™) demonstrate this principle. Additionally, this assay may be used for measuring ligand binding to receptors using radiolabeled ligands. In a similar manner, the radiolabeled bound ligand is centrifuged to the bottom of the well and activates the scintillant. The [³⁵S]GTPγS assay results parallel the results obtained in traditional second messenger assays of receptors.

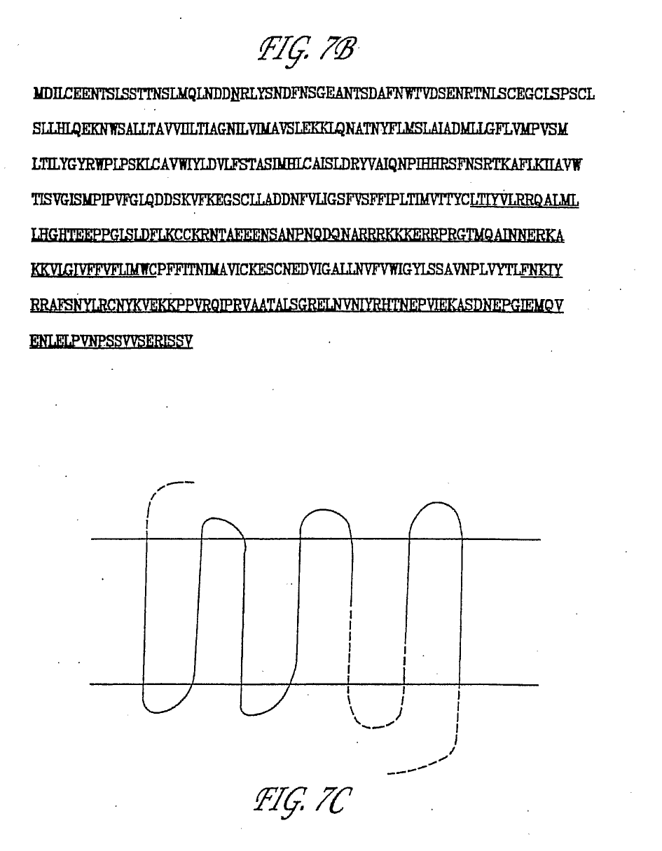

As shown in FIG. 10, serotonin stimulates the binding of [³⁵S]GTPγS to the endogenous human 5-HT_{2C} receptor, while mianserin inhibits this response; furthermore, mianserin acts as a partial inverse agonist by inhibiting the basal constitutive binding of [³⁵S]GTPγS to membranes expressing the endogenous human 5-HT_{2C} receptor. As expected, there is no agonist response in the absence of GDP since there is no GDP present to exchange for [³⁵S]GTPγS. Not only does this assay system demonstrate the response of the native 5HT_{2C} receptor, but it also measures the constitutive activation of other receptors.

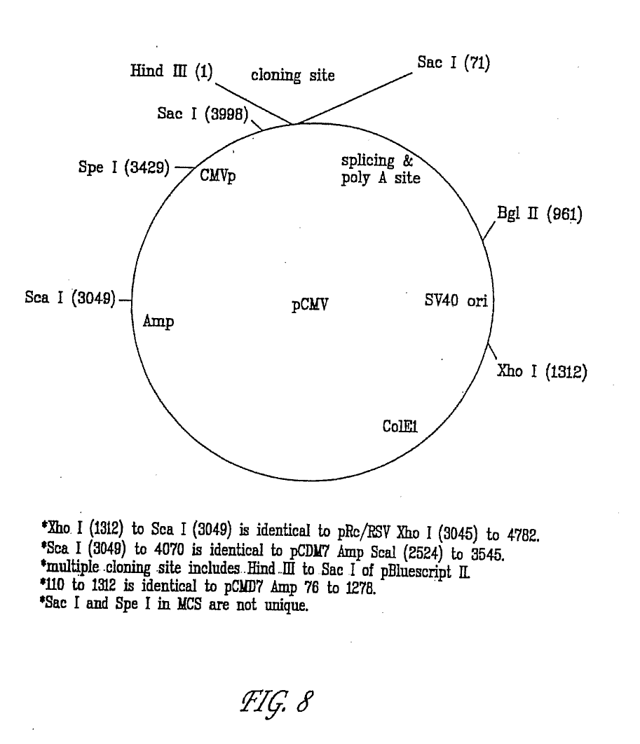

FIGS. 11A and 11B demonstrate that level of binding of [³⁵S]GTPγS to membranes prepared from 293T cells expressing the native human 5-HT_{2C} receptor or the AP-1 receptor was enhanced relative to the level of binding of [³⁵S]GTPγS to membranes prepared from 293T cells expressing control vector alone. The total protein concentration used in the assay affects the total amount of [³⁵S]GTPγS binding for each receptor. The c.p.m. differential between the CMV transfected and the constitutively active mutant receptor increased from approximately 1000 c.p.m at 10 μg/well to approximately 6-8000 c.p.m. at 75 μg/well protein concentration, as shown in FIG. 11.

The AP-1 receptor showed the highest level of constitutive activation followed by the wild type receptor, which also showed enhanced [³⁵S]GTPγS binding above basal. This is consistent with the ability of the endogenous human 5-HT_{2C} receptor to accumulate intracellular IP₃ in the absence of 5HT stimulation (Example 5) and is also consistent with published data claiming that the endogenous human 5-HT_{2C} receptor has a high natural basal activity. Therefore, the AP-1 receptor demonstrates that constitutive activity may be measured by proximal [³⁵S]GTPγS binding events at the membrane interface.

### Example 4

### Serotonin Receptor Agonist/Antagonist Competitive Binding Assay

Membranes were prepared from transfected COS-7 cells (see Example 2) by homogenization in 20 mM HEPES and 10 mM EDTA, pH 7.4 and centrifuged at 49,000×g for 15 min. The pellet was resuspended in 20 mM HEPES and 0.1 mM EDTA, pH 7.4, homogenized for 10 sec. using a Polytron homogenizer (Brinkman) at 5000 rpm and centrifuged at 49,000×g for 15 min. The final pellet was resuspended in 20 mM HEPES and 10 mM MgCl₂, pH 7.4, homogenized for 10 sec. using polytron homogenizer (Brinkman) at 5000 rpm.

Assays were performed in triplicate 200 μl volumes in 96 well plates. Assay buffer (20 mM HEPES and 10 mM MgCl₂, pH 7.4) was used to dilute membranes, ³H-LSD, ³H-mesulergine, serotonin (used to define non-specific for LSD binding) and mianserin (used to define non-specific for mesulergine binding). Final assay concentrations consisted of 1 nM ³H-LSD or 1 nM ³H-mesulergine, 50 μg membrane protein and 100 μm serotonin or mianserin. LSD assays were incubated for 1 hr at 37° C., while mesulergine assays were incubated for 1 hr at room temperature. Assays were terminated by rapid filtration onto Wallac Filtermat Type B with ice cold binding buffer using Skatron cell harvester. The radioactivity was determined in a Wallac 1205 BetaPlate counter.

### Example 5

### Intracellular IP₃ Accumulation Assay

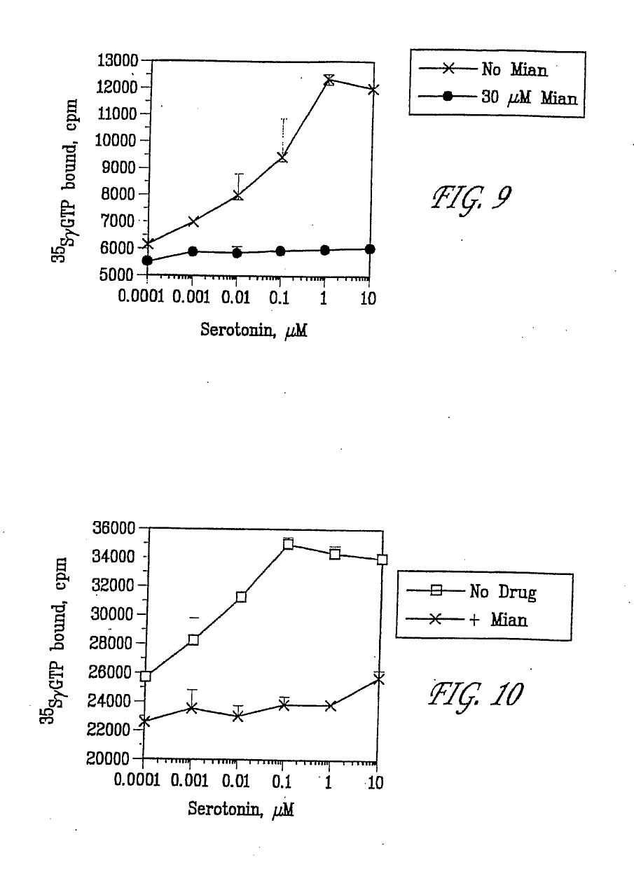

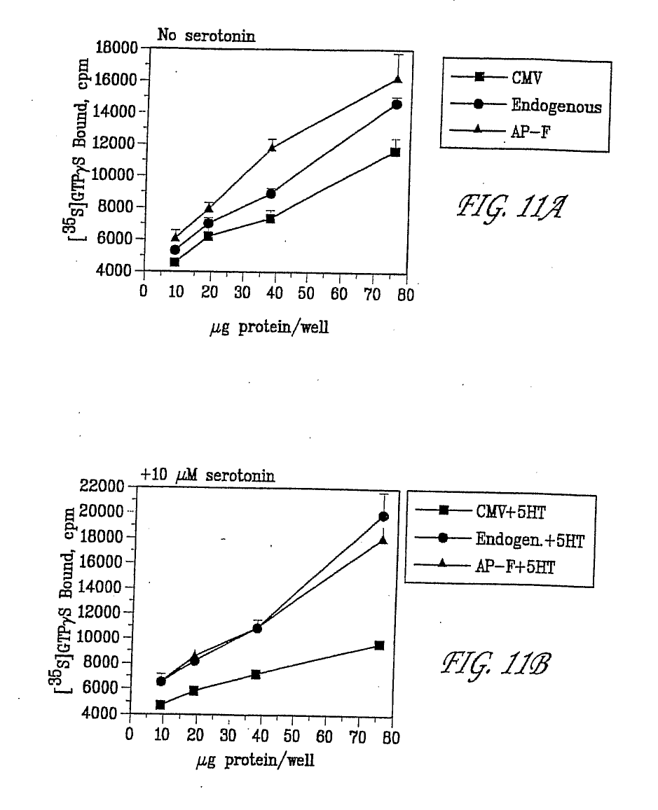

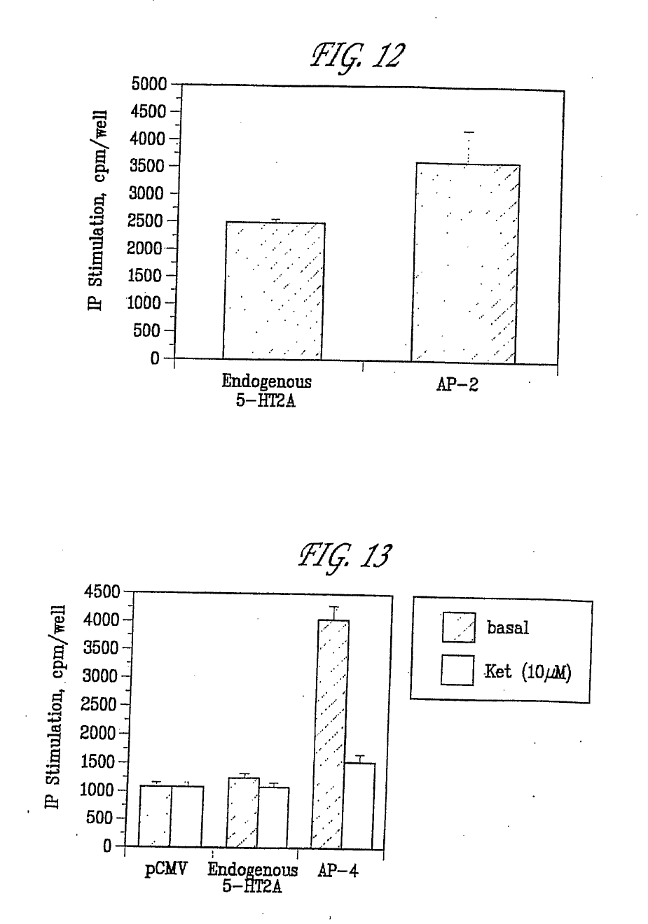

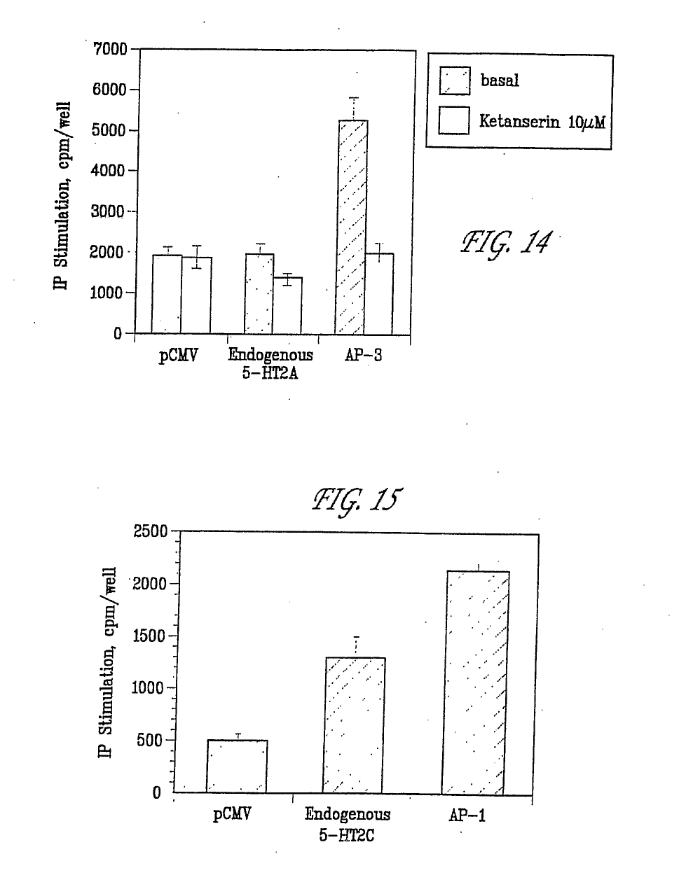

For the IP₃ accumulation assay, a transfection protocol different from the protocol set forth in Example 3 was utilized. In the following example, the protocols used for days 1-3 were slightly different for the data generated for FIGS. 12 and 14 and for FIGS. 13 and 15; the protocol for day 4 was the same for all conditions.

A. COS-7 and 293 Cells

On day one, COS-7 cells or 293 cells were plated onto 24 well plates, usually 1×10⁵ cells/well or 2×10⁵ cells/well, respectively. On day two, the cells were transfected by first mixing 0.25 ug DNA (see Example 2) in 50 μl serum-free DMEM/well and then 2 μl lipofectamine in 50 μl serum-free DMEM/well. The solutions (“transfection media”) were gently mixed and incubated for 15-30 minutes at room temperature. The cells were washed with 0.5 ml PBS and then 400 μl of serum free media was mixed with the transfection media and added to the cells. The cells were then incubated for 3-4 hours at 37° C./5% CO₂. Then the transfection media was removed and replaced with 1 ml/well of regular growth media. On day 3, the media was removed and the cells were washed with 5 ml PBS followed by aspiration. Then 2 ml of trypsin (0.05%) is added per plate. After 20-30 seconds, warm 293 media is added to plates, cells are gently resupended, and cells are counted. Then a total of 55,000 cells are added to sterile poly-D-lysine treated 96 well microtiter plates and cells are allowed to attach over a six-hour incubation in an incubator. Then media is aspirated and 0.1 mL inositol-free/serum-free media (GIBCO BRL) was added to each well with 0.25 μCi of ³H-myo-inositol/well and the cells were incubated for 16-18 hours overnight at 37° C./5% CO₂. Protocol A.

B. 293 Cells

On day one, 13×10⁶ 293 cells per 150 mm plate were plated out. On day two, 2 ml of serum OptimemI (Invitrogen Corporation) is added per plate followed by addition of 60 μL of lipofectamine and 16 μg of DNA (e.g., pCMV vector; pCMV vector with receptor cDNA; etc.). Note that lipofectamine must be added to the OptimemI and mixed well before addition of DNA. While complexes between lipofectamine and the DNA are forming, media is carefully aspirated and cells are gently rinsed with 5 ml of OptimemI media followed by careful aspiration. Then 12 ml of OptimemI is added to each plate and 2 ml of transfection solution is added followed by a 5 hour incubation at 37° C. in a 5% CO₂ incubator. Plates are then carefully aspirated and 25 mL of Complete Media are added to each plate and cells are then incubated until used. On day 3, cells are trypsinized with 2 ml of 0.05% trypsin for 20-30 seconds followed by addition of 10 mL of warmed media, gently titurated to dissociate cells, and then 13 additional ml of warmed media is gently added. Cells are then counted and then 55,000 cells are added to 96-well sterile poly-D-lysine trated plates. Cells are allowed to attach over a six hour incubation at 37° C. in a 5% CO₂ incubator. Media is then carefully aspirated and 100 μL of warm inositol-free media plus 0.5 μCi ³H-inositol is added to each well and the plates are incubated for 18-20 hours at 37° C. in a 5% CO₂ incubator.

On day 4, media is carefully aspirated and then 0.1 ml of assay medium is added containing inositol-free/serum free media, 10 μM pargyline, 10 mM lithium chloride, and test compound at indicated concentrations. The plates were then incubated for three hours at 37° C. and then wells are carefully aspirated. Then 200 μL of ice-cold 0.1M formic acid is added to each well. Plates can then be frozen at this point at −80° C. until further processed. Frozen plates are then thawed over the course of one hour, and the contents of the wells (approximately 220 μL) are placed over 400 μL of washed ion-exchange resin (AG 1-X8) contained in a Multi Screen Filtration plate and incubated for 10 minutes followed by filtration under vacuum pressure. Resin is then washed nine times with 200 μL of water and then tritiated inositol phosphates are eluted into a collecting plate by the addition of 200 ul of 1M ammonium formate and an additional 10 minute incubation. The elutant is then transferred to 20 ml scintillation vials, 8 mL of SuperMix or Hi-Safe scintillation cocktails is added, and vials are counted for 0.5-1 minutes in a Wallac 1414 scintilation counter.

FIG. 12 is an illustration of IP3 production from AP-2, the human 5-HT_{2A} receptor which was mutated using the same point mutation as set forth in Casey, which rendered the rat receptor constitutively active. The results represented in FIG. 12, support the position that when the point mutation shown to activate the rat receptor is introduced into the human receptor, little activation of the receptor is obtained that would allow for appropriate screening of candidate compounds, with the response being only moderately above that of the endogenous human 5-HT_{2A} receptor. Generally, a response of at least 2× above that of the endogenous response is preferred.

FIG. 13 provides an illustration comparing IP₃ production from endogenous 5-HT_{2A} receptor and the AP-4 mutation. The results illustrated in FIG. 13 support the position that when the novel mutation disclosed herein is utilized, a robust response of constitutive IP3 accumulation is obtained (e.g., over 2× that of the endogenous receptor).

FIG. 14 provides an illustration of IP3 production from AP-3. The results illustrated in FIG. 14 support the position that when the novel mutation disclosed herein is utilized, a robust response of constitutive IP3 accumulation is obtained.

FIG. 15 provides bar-graph comparisons of IP3 accumulation between endogenous human 5-HT_{2C} receptor and AP-1. Note that the endogenous receptor has a high degree of natural constitutive activity relative to the control CMV transfected cells (i.e., the endogenous receptor appears to be constitutively activated).

### Example 6

### In Vitro Binding of 5HT_{2A} Receptor

### Animals:

Animals (Sprague-Dawley rats) were sacrificed and brains were rapidly dissected and frozen in isopentane maintained at −42° C. Horizontal sections were prepared on a cryostat and maintained at −20° C.

### LSD Displacement Protocol:

Lysergic acid diethylamide (LSD) is a potent 5HT_{2A} receptor and dopamine D2 receptor ligand. An indication of the selectivity of compounds for either or both of these receptors involves displacement of radiolabeled-bound LSD from pre-treated brain sections. For these studies, radiolabeled ¹²⁵I-LSD (NEN Life Sciences, Boston, Mass., Catalogue number NEX-199) was utilized; spiperone (RBI, Natick, Mass. Catalogue number s-128) a 5HT_{2A} receptor and dopamine D2 receptor antagonist, was also utilized. Buffer consisted of 50 nanomolar TRIS-HCl, pH 7.4.

Brain sections were incubated in (a) Buffer plus 1 nanomolar ¹²⁵I-LSD; (b) Buffer plus 1 nanomolar ¹²⁵I-LSD and 1 micromolar spiperone; or Buffer plus 1 nanomolar ¹²⁵I-LSD and 1 micromolar Compound S-1610, [3-(4-Bromo-2-methyl-2H-pyrazol-3-yl)-phenyl]-carbamic acid 4-methoxy-phenyl ester, for 30 minutes at room temperature. S-1610 is a modulator of 5HT_{2A} identified as an early lead compound by the Inventors. Sections were then washed 2×10 minutes at 4° C. in Buffer, followed by 20 seconds in distilled H₂O. Slides were then air-dried.

After drying, sections were apposed to x-ray film (Kodak Hyperfilm) and exposed for 4 days.

### Analysis:

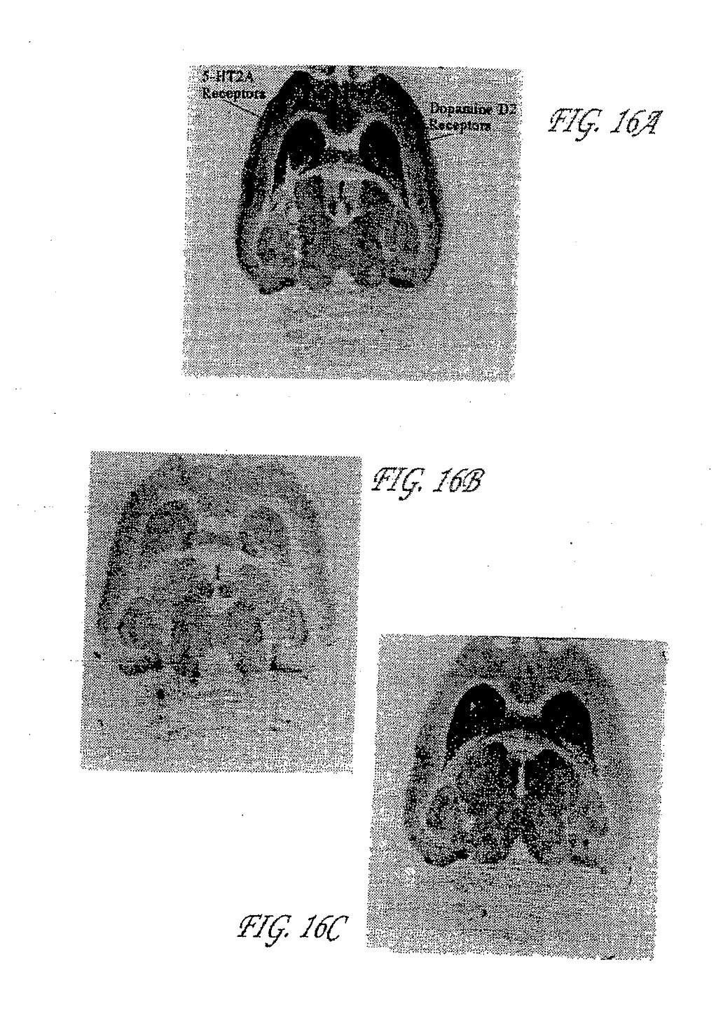

FIGS. 16A-16C provide grey-scale representative autoradiographic sections from this study. FIG. 16A evidences darker bands (derived from ¹²⁵I-LSD binding) primarily in both the fourth layer of the cerebral cortex (primarily 5HT_{2A} receptors), and the caudate nucleus (primarily dopamine D2 receptors and some 5HT_{2A} receptors). As can be seen from FIG. 16B, spiperone, which is a 5HT_{2A} and dopamine D2 antagonist, displaces the I¹²⁵-LSD from these receptors on both the cortex and the caudate. As can be further seen from FIG. 16C, Compound S-1610 appears to selectively displace the ¹²⁵I-LSD from the cortex (5HT_{2A}) and not the caudate (dopamine D2).

### Example 7

Screening Compounds Reported as having 5-HT_{2C} Antagonist Activity Against Non-Endogenous, Constitutively Activated Human Serotonin Receptor: AP-1

A final concentration of 12.5 μg membranes prepared from COS-7 cells (see Example 2) transiently expressing constitutively active mutant human 5HT_{2C} receptor AP-1 were incubated with binding buffer (20 mM HEPES, pH 7.4, 100 mM NaCl, 20 mM MgCl₂.6H₂O, 0.2% saponin, and 0.2 mM ascobate), GDP (1 μM) and compound in a 96-well plate format for a period of 60 minutes at ambient room temperature. Plates were then centrifuged at 4,000 rpm for 15 minutes followed by aspiration of the reaction mixture and counting for 1 minute in a Wallac™ MicroBeta plate scintillation counter. A series of compounds reported to possess 5HT_{2C} antagonist activity were determined to be active in the [³⁵S]GTPγS binding assay using AP-1. IC₅₀ determinations were made for these commercially available compounds (RBI, Natick, Mass.). Results are summarized in TABLE 5. For each determination, eight concentrations of test compounds were tested in triplicate. The negative control in these experiments consisted of AP-1 receptor without test compound addition, and the positive control consisted of 12.5 μg/well of cell membrane COS-7 cells transfected with the pCMV vector alone (i.e., without AP-1 receptor).

TABLE 5

| | | IC₅₀ (nM) in |
|---|---|---|
| | Known | GTP-γ-[³⁵S] |
| Test Compound | Pharmacology | Assay |
| Metergoline | 5HT_{2C} antagonist | 32.0 |
| Mesulergine | 5HT_{2C} antagonist | 21.2 |
| Methysergide | 5HT_{2C} antagonist | 6.1 |
| Methiothepin | 5HT₁ antagonist | 20.4 |
| Normethylclozapin | 5HT_{2C} antagonist | 21.4 |
| Fluoxetine | 5HT reuptake | 114.0 |
| | inhibitor | |
| Ritanserin | 5HT_{2C} antagonist | 19.4 |

The IC₅₀ results confirm that the seven tested compounds showed inverse agonist activity at the AP-1 receptor.

### Example 8

### Receptor Binding Assay

In addition to the methods described herein, another means for evaluating a test compound is by determining binding affinities to the 5-HT_{2A} receptor. This type of assay generally requires a radiolabeled ligand to the 5-HT_{2A} receptor. Absent the use of known ligands for the 5-HT_{2A} receptor and radiolabels thereof, compounds of the present invention can be labelled with a radioisotope and used in an assay for evaluating the affinity of a test compound to the 5-HT_{2A} receptor.

A radiolabeled 5-HT_{2A} compound of Formula (I) can be used in a screening assay to identify/evaluate compounds. In general terms, a newly synthesized or identified compound (i.e., test compound) can be evaluated for its ability to reduce binding of the “radiolabeled compound of Formula (I)” to the 5-HT_{2A} receptor. Accordingly, the ability to compete with the “radiolabeled compound of Formula (I)” or Radiolabeled 5-HT_{2A} Ligand for the binding to the 5-HT_{2A} receptor directly correlates to its binding affinity of the test compound to the 5-HT_{2A} receptor.

### Assay Protocol for Determining Receptor Binding for 5-HT_{2A}:

A. 5-HT_{2A} Receptor Preparation

293 cells (human kidney, ATCC), transiently transfected with 10 μg human 5-HT_{2A} receptor and 60 ul Lipofectamine (per 15-cm dish), are grown in the dish for 24 hours (75% confluency) with a media change and removed with 10 ml/dish of Hepes-EDTA buffer (20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 ul of appropriately diluted membranes (diluted in assay buffer containing 50 mM Tris HCl (pH 7.4), 10 mM MgCl₂, and 1 mM EDTA; 5-50 μg protein) is added to 96-well polyproylene microtiter plates followed by addition of 100 μl of assay buffer and 50 μl of Radiolabeled 5-HT_{2A} Ligand. For nonspecific binding, 50 μl of assay buffer is added instead of 100 μl and an additional 50 μl of 10 μM cold 5-HT_{2A} is added before 50 μl of Radiolabeled 5-HT_{2A} Ligand is added. Plates are then incubated at room temperature for 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. Then, the bottom of the filtration plate are sealed, 50 μl of Optiphase Supermix is added to each well, the top of the plates are sealed, and plates are counted in a Trilux MicroBeta scintillation counter. For compound competition studies, instead of adding 100 μl of assay buffer, 100 μl of appropriately diluted test compound is added to appropriate wells followed by addition of 50 μl of Radiolabeled 5-HT_{2A} Ligand.

C. Calculations

The test compounds are initially assayed at 1 and 0.1 μM and then at a range of concentrations chosen such that the middle dose would cause about 50% inhibition of a Radio-5-HT_{2A} Ligand binding (i.e., IC₅₀). Specific binding in the absence of test compound (B_{O}) is the difference of total binding (B_{T}) minus non-specific binding (NSB) and similarly specific binding (in the presence of test compound) (B) is the difference of displacement binding (B_{D}) minus non-specific binding (NSB). IC₅₀ is determined from an inhibition response curve, logit-log plot of % B/B_{O} vs concentration of test compound.

Kᵢ is calculated, for example, by the Cheng and Prustoff transformation:

Kᵢ=IC₅₀/(1+[L]/K_{D})

where [L] is the concentration of a Radio-5-HT_{2A} Ligand used in the assay and K_{D} is the dissociation constant of a Radio-5-HT_{2A} Ligand determined independently under the same binding conditions.

### Example 9

### Inverse Agonist Activity of Compounds of the Present Invention in the IP₃ Accumulation Assay

Certain compounds of the present invention evidencing inverse agonist activity at 5-HT_{2A} and their corresponding IC₅₀ values in the IP₃ Accumulation Assay are shown in TABLE 6.

TABLE 6

| | 5-HT_{2A} (IC₅₀)* |
|---|---|
| Compound No. | IP₃ Accumulation Assay (nM) |
| 20 | 0.45 |
| 60 | 1.10 |
| 61 | 8.57 |
| 79 | 13.0 |
| 84 | 12.2 |

*Reported values are averages of at least two trials.

The majority of the other compounds provided by way of illustration in Table A were tested at least once and evidenced inverse agonist activity at 5-HT_{2A} with IC₅₀ values in the IP₃ Accumulation Assay of less than about 10 μM.

### Example 10

### Blood Brain Barrier Model

The ability of a compound of the invention to cross the blood-brain barrier can be shown using brain-derived cells. One method that is envisioned, by way of illustration and not limitation, is to use the blood/brain barrier model of Dehouck et al. [J Neurochem (1990) 54:1798-801; hereby incorporated by reference in its entirety] that uses a co-culture of brain capillary endothelial cells and astrocytes.

Bovine capillary endothelial (BBCE) cells are isolated and characterized as described by Meresse et al. [J Neurochem (1989) 53:1363-1371; hereby incorporated by reference in its entirety]. In brief, after isolation by mechanical homogenization from one hemisphere of bovine brain, microvessels are seeded onto dishes coated with an extracellular matrix secreted by bovine corneal endothelial cells. Five days after seeding, the first endothelial cells migrate out from the capillaries and begin to form microcolonies. When the colonies are sufficiently large, the five largest islands are trypsinized and seeded onto 35-mm-diameter gelatin-coated dishes (one clone per dish) in the presence of Dulbecco's modified Eagle's medium (DMEM) supplemented with 15% calf serum (Seromed), 3 mM glutamine, 50 μg/ml of gentamicin, 2.5 μg/ml of amphotericin B (Fungizone), and bovine fibroblast growth factor (1 ng/ml added every other day). Endothelial cells from one 35-mm-diameter dish are harvested at confluence and seeded onto 60-mm-diameter gelatin-coated dishes. After 6-8 days, confluent cells are subcultured at the split ratio of 1:20. Cells at the third passage (˜100 dishes) are stored in liquid nitrogen.

Primary cultures of astrocytes are made from newborn rat cerebral cortex. After the meninges have been cleaned off, the brain tissue is forced gently through a nylon sieve as described by Booher and Sensenbrenner [Neurobiology (1972) 2:97-105; hereby incorporated by reference in its entirety]. DMEM supplemented with 10% fetal calf serum (Seromed), 2 mM glutamine, and 50 μg/ml of gentamicin is used for the dissociation of cerebral tissue and development of astrocytes.

Culture plate inserts (Millicell-CM; pore size, 0.4 μM; diameter, 30 mm; Millipore) are coated on both sides with rat tail collagen prepared by a modification of the method of Bornstein [Lab Invest (1958) 7:134-139; hereby incorporated by reference in its entirety].

Astrocytes are plated at a concentration of 2.5×10⁵ cells/ml on the bottom side using the filter upside down. After 8 days, filters are properly positioned, and the medium is changed twice a week. Three weeks after seeding, cultures of astrocytes become stabilized. Then, BBCE cells, frozen at passage 3, are recultured on a 60-mm-diameter gelatin-coated dish. Confluent cells are trypsinized and plated on the upper side of the filters at a concentration of 4×10⁵ cells. The medium used for the coculture is DMEM supplemented with 15% calf serum 2 mM glutarnine, 50 μg/ml of gentamicin, and 1 ng/ml of bovine fibroblast growth factor added every other day. Under these conditions, BBCE cells form a confluent monolayer in 8 days.

Culture plates are set into six-well plates with 2 ml of buffer added to the upper chamber and 2 ml added to the plate containing the inserts. The six-well plates are placed in a shaking water bath at 37° C. The compound of the invention is added to the upper chamber, and 100 μl is removed from the lower chamber at various time points. In certain embodiments, the test compound is radiolabeled. In certain embodiments, the radiolabel is ³H or ¹⁴C. In some embodiments, the final time point is about 20 min, about 30 min, about 40 min, about 50 min, about 60 min, about 70 min, about 80 min or about 90 min. The percentage of total test compound present in the lower chamber at the various time points is determined. Leucine is used as a permeability positive control. Inulin is used as a permeability negative control.

### Example 11

### Efficacy of Compounds of the Invention in the Attenuation of DOI-Induced Hypolocomotion in Rats.

In this Example, compounds of the invention, such as Compound 1 and Compound 26, were tested for inverse agonist activity by determining whether these compounds could attenuate DOI-induced hypolocomotion in rats in a novel environment. DOI is a potent 5HT_{2A}/5HT_{2C} receptor agonist that crosses the blood-brain barrier.

Animals

Male Sprague-Dawley rats (Harlan, San Diego, Calif.) weighing between 200-300 g were used for all tests. Rats were housed three to four per cage. These rats were naive to experimental testing and drug treatment. Rats were handled one to three days before testing to acclimate them to experimental manipulation. Rats were fasted overnight prior to testing.

Compounds

(R)-DOI HCl (C₁₁H₁₆INO₂HCl) was obtained from Sigma-Aldrich, and was dissolved in 0.9% saline. Compounds of the invention were synthesized at Arena Pharmaceuticals Inc. and were dissolved in 100% PEG400. DOI was injected s.c. in a volume of 1 ml/kg, while compounds of the invention were administered p.o. in a volume of 2 ml/kg.

Procedure

The “Motor Monitor” (Hamilton-Kinder, Poway, Calif.) was used for all activity measurement. This apparatus recorded rears using infrared photobeams.

Locomotor activity testing was conducted during the light cycle (0630-1830) between 9:00 a.m. and 4:00 p.m. Animals were allowed 30 min acclimation to the testing room before testing began.

In determining the effects of compounds of the invention on DOI-induced hypoactivity, animals were first injected with vehicle or the compound of the invention (50 μmol/kg) in their home cages. Sixty minutes later, saline or DOI (0.3 mg/kg salt) was injected. 10 min after DOI administration, animals were placed into the activity apparatus and rearing activity was measured for 10 minutes.

Statistics and Results

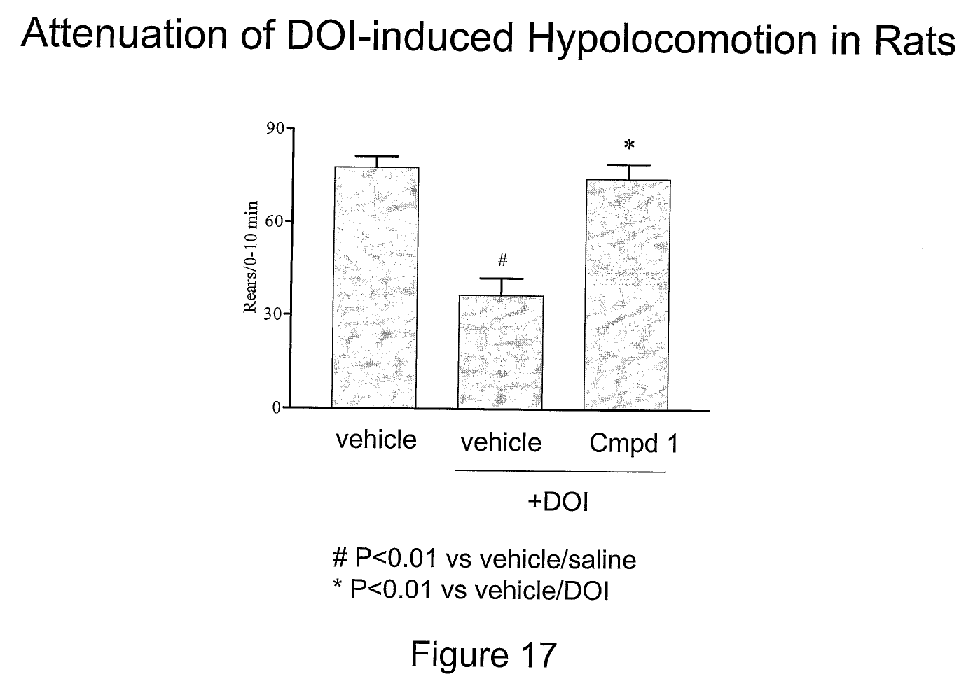

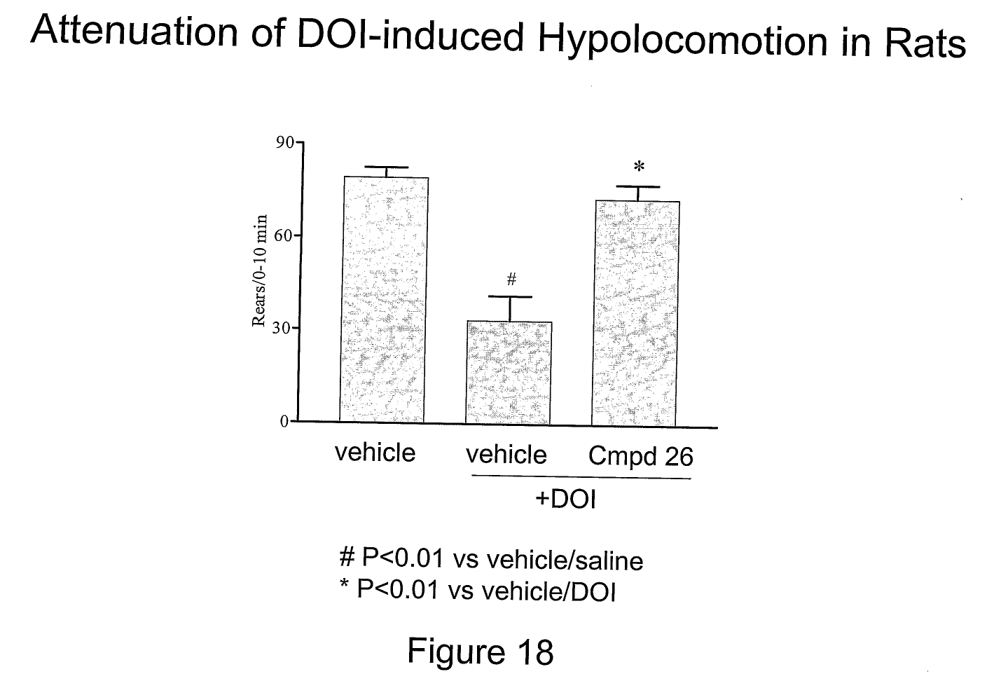

Results (total rears over 10 minutes) were analyzed by t-test. P<0.05 was considered significant. As shown in FIG. 17, Compound 1 attenuated DOI-induced hypolocomotion in rats. In addition, as shown in FIG. 18, Compound 26 also attenuated DOI-induced hypolocomotion in rats.

### Example 12

### Serotonin 5-HT2A Receptor Occupancy Studies in Monkey

In this Example, the 5HT_{2A} receptor occupancy of a compound of the invention, Compound 1, was measured. The study was carried out in rhesus monkeys using PET and ¹⁸F-altanserin.

Radioligand:

The PET radioligand used for the occupancy studies was ¹⁸F-altanserin. Radiosynthesis of ¹⁸F-altanserin is achieved in high specific activities and is suitable for radiolabeling 5HT_{2A} receptors in vivo (see Staley et al., Nucl. Med. Biol., 28:271-279 (2001) and references cited within). Quality control issues (chemical and radiochemical purity, specific activity, stability etc) and appropriate binding of the radioligand were verified in rat brain slices prior to use in PET experiments.

Drug Doses and Formulations:

Briefly, the radiopharmaceutical was dissolved in sterile 0.9% saline, pH approx 6-7. The compounds of the invention (Compound 1) were dissolved in 60% PEG 400-40% sterile saline on the same day of the PET experiment.

Serotonin 5HT_{2A} occupancy studies in humans have been reported for M100,907 (Grunder et al., Neuropsychopharmacology, 17:175-185 (1997), and Talvik-Lofti et al., Psychophamacology, 148:400-403 (2000)). High occupancies of the 5HT_{2A} receptors have been reported for various oral doses (doses studied ranged from 6 to 20 mg). For example, an occupancy of >90% was reported for a dose of 20 mg (Talvik-Lofti et al., supra), which translates to approx. 0.28 mg/kg. It may therefore be anticipated that an i.v. dose of 0.1 to 0.2 mg/kg of M100,907 is likely to provide high receptor occupancy. A 0.5 mg/kg dose of Compound 1 was used in these studies.

PET Experiments

The monkey was anesthetized by using ketamine (10 mg/kg) and was maintained using 0.7 to 1.25% isoflurane. Typically, the monkey had two i.v. lines, one on each arm. One i.v. line was used to administer the radioligand, while the other line was used to draw blood samples for pharmacokinetic data of the radioligand as well as the cold drugs. Generally, rapid blood samples were taken as the radioligand is administered which then taper out by the end of the scan. A volume of approximately 1 ml of blood was taken per time point, which was spun down, and a portion of the plasma was counted for radioactivity in the blood.

An initial control study was carried out in order to measure baseline receptor densities. PET scans on the monkey were separated by at least two weeks. Unlabeled drug (Compound 1) was administered intravenously, dissolved in 80% PEG 400:40% sterile saline.

PET Data Analysis

PET data were analyzed by using cerebellum as the reference region and using the distribution volume region (DVR) method. This method has been applied for the analysis of ¹⁸F-altanserin PET data in nonhuman primate and human studies (Smith et al., Synapse, 30:380-392 (1998).

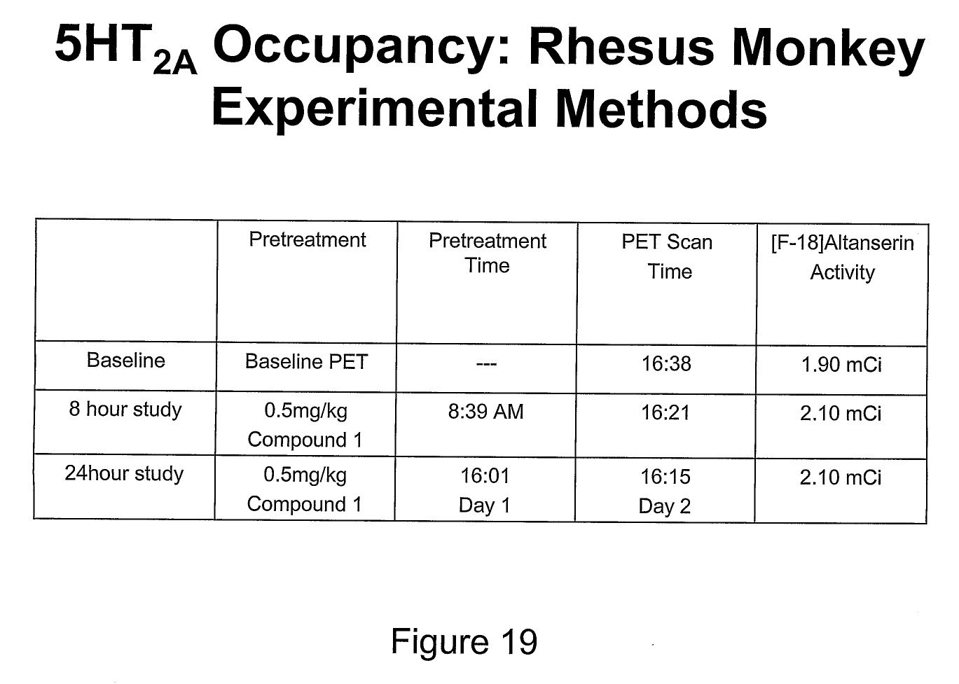

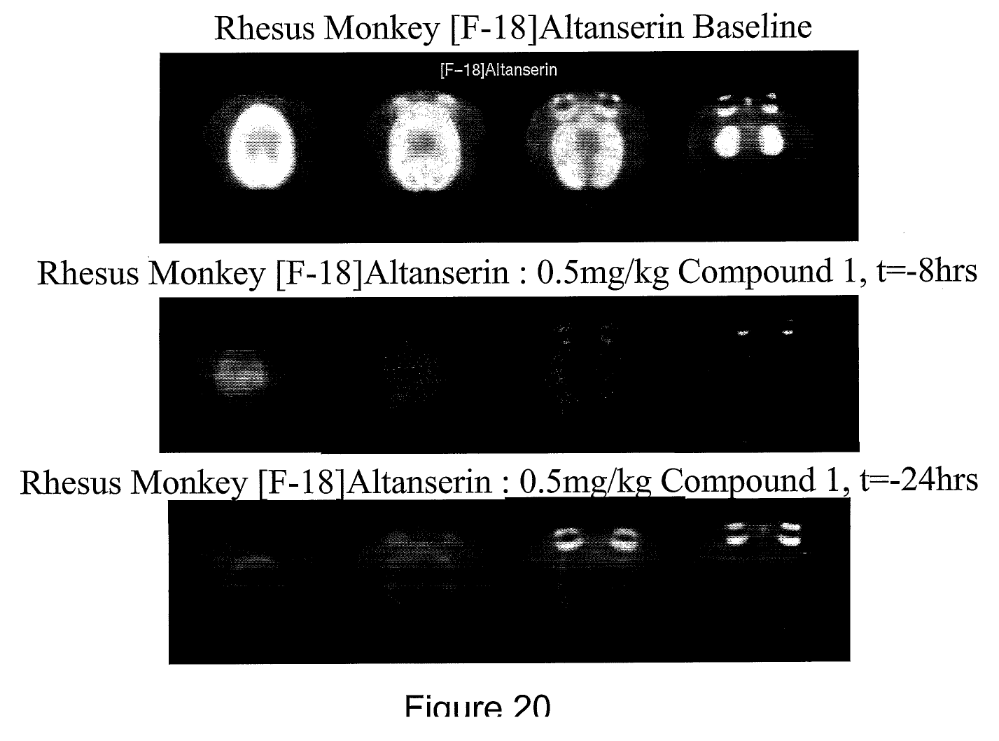

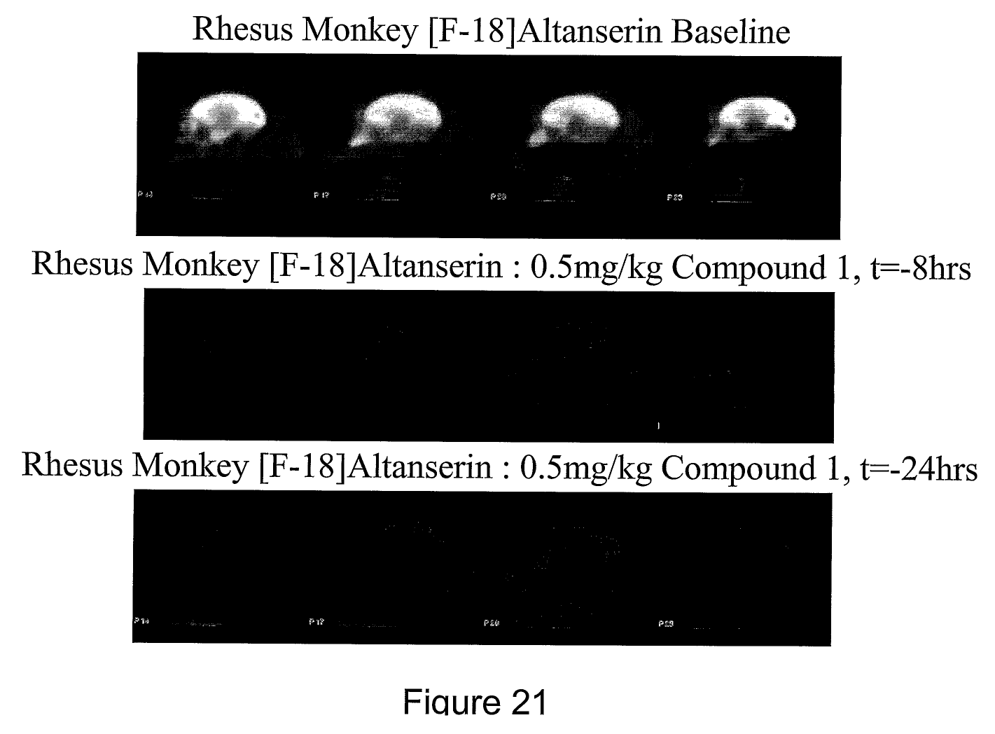

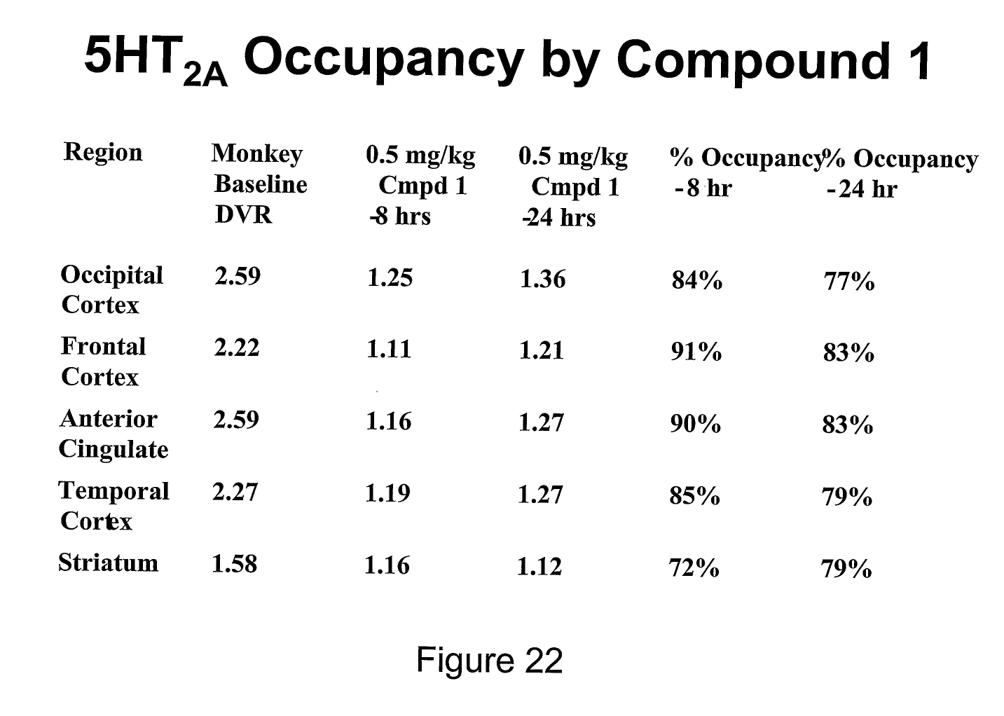

The 5HT_{2A} occupancy (rhesus monkey experimental methods) of Compound 1 is shown in FIGS. 19-22. The results of both an 8 hour and 24 hour study are shown. The test compound was administered via i.v. infusion in 5.0 ml of 80% PEG400. For the 8 hour study, venous blood samples were drawn at 5 minutes post Compound 1 and 15 minutes before PET scan. For the 24 hour study, venous blood samples were drawn at 5 minutes post Compound 1 and 10 minutes before PET scan.

The results show that 5HT_{2A} receptor occupancy of Compound 1 at the dose of 0.5 mg/kg after 8 hours following drug administration was approximately 90% in the cortical regions, which is an area of high 5HT_{2A} receptor density. This occupancy dropped to approximately 80% at 24 hours post-injection although no measurable test drug concentrations were apparent in plasma samples after 8 hours.

### Example 13

### Efficacy of Compounds of the Invention in the Inhibition of JC Virus Infection of Human Glial Cells

A compound of the invention can be shown to inhibit JC virus infection of human glial cells using the in vitro model of Elphick et al. [Science (2004) 306:1380-1383], essentially as described briefly here.

Cells and JC Virus

The human glial cell line SVG (or a suitable subclone thereof, such as SVG-A) is used for these experiments. SVG is a human glial cell line established by transformation of human fetal glial cells by an origin defective SV40 mutant Major et al., Proc. Natl. Acad. Sci. USA (1985) 82:1257-1261]. SVG cells are cultured in Eagle's minimum essential medium (Mediatech Inc., Herndon, Va.) supplemented with 10% heat-inactivated fetal bovine serum, and kept in a humidified 37° C. 5% CO₂ incubator.

The Mad-1/SVEΔ strain of JC virus [Vacante et al., Virology (1989) 170:353-361] is used for these experiments. While the host range of JC virus is typically limited to growth in human fetal glial cells, the host range of Mad-1/SVEΔ extends to human kidney and monkey cell types. Mad-1/SVEΔ is propagated in HEK cells. Virus titer is measured by hemagglutination of human type O erythrocytes.

Assay for Inhibition of JC Virus Infection

SVG cells growing on coverslips are pre-incubated at 37° C. for 45 min with or without the compound of the invention diluted in media containing 2% FCS. By way of illustration and not limitation, the compound of the invention is used at a concentration of about 1 nM to about 100 μM, at a concentration of about 10 nM to about 100 μM, at a concentration of about 1 nM to about 10 μM, or at a concentration of about 10 nM to about 10 μM.

JC virus (Mad-1/SVEΔ) is then added at an MOI of 1.0 and the cells are incubated for 1 hr at 37° C. in the continued presence of the compound of the invention. The cells are then washed 3× in PBS and fed with growth media containing the compound of the invention. At 72 hr post-infection, V antigen positive cells are scored by indirect immunofluorescence (see below). Controls include the addition of the compound of the invention at 24 and 48 h post-infection. The percentage of infected cells in untreated cultures is set at 100%.

Indirect Immunofluorescence

For indirect immunofluorescence analysis of V antigen expression, SVG cells growing on coverslips are fixed in ice cold acetone. To detect V antigen expression, the cells are then incubated for 30 min at 37° C. with a 1:10 dilution of hybridoma supernatant from PAB597. The PAB597 hybridoma produces a monoclonal antibody against the SV40 capsid protein VP1 which has been shown to cross-react with JC virus VP1. The cells are then washed and incubated with goat anti-mouse Alexa Fluor 488 secondary antibody for an additional 30 min. After a final wash, the cells are counterstained with 0.05% Evan's blue, mounted onto glass slides using 90% glycerol in PBS and visualized on Nikon E800 epifluorescent scope. Images are captured using a Hamamatsu digital camera and analyzed using Improvision software.

Those skilled in the art will recognize that various modifications, additions, substitutions, and variations to the illustrative examples set forth herein can be made without departing from the spirit of the invention and are, therefore, considered within the scope of the invention. All documents referenced above, including, but not limited to, printed publications, and provisional and regular patent applications, are incorporated herein by reference in their entirety.

1. A method of prophylaxis or treatment of progressive multifocal leukoencephalopathy comprising administering to an individual in need thereof a therapeutically effective amount of a compound or of a pharmaceutical composition comprising the compound and a pharmaceutically acceptable carrier, wherein the compound is a compound of Formula (I): 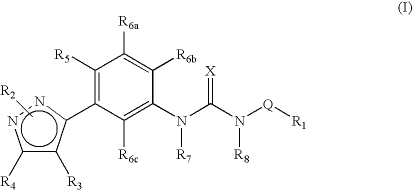
or a pharmaceutically acceptable salt, hydrate or solvate thereof;
wherein:
i) R₁ is aryl or heteroaryl each optionally substituted with R₉, R₁₀, R₁₁, R₁₂, R₁₃, R₁₄, and R₁₅ each selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F, Cl, or Br; and wherein said C₂₋₆ alkenyl, C₁₋₆ alkyl, C₂₋₆ alkynyl, C₁₋₆ alkylamino, C₁₋₆ alkylimino, C₂₋₈ dialkylamino, heterocyclic, and phenyl are each optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;
ii) R₂ is selected from the group consisting of H, C₁₋₆ alkyl, C₂₋₆ alkenyl, C₂₋₆ alkynyl and C₃₋₇ cycloalkyl;
iii) R₃ is selected from the group consisting of H, C₂₋₆ alkenyl, C₁₋₆ alkyl, C₁₋₆ alkylcarboxamide, C₂₋₆ alkynyl, C₁₋₆ alkylsulfonamide, carbo-C₁₋₆-alkoxy, carboxamide, carboxy, cyano, C₃₋₇ cycloalkyl, C₂₋₈ dialkylcarboxamide, halogen, heteroaryl and phenyl; and wherein each of said C₂₋₆ alkenyl, C₁₋₆ alkyl, C₂₋₆ alkynyl, C₁₋₆ alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and sulfonamide;
iv) R₄ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;
v) R₅ is selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide, wherein said C₁₋₆ alkoxy group is optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, amino, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and phenyl, and wherein said amino and phenyl substituents are each optionally substituted with 1 to 5 further substituents selected from the group consisting of halogen and carbo-C₁₋₆-alkoxy;
vi) R₆ₐ, R_{6b}, and R_{6c} are each independently selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;
vii) R₇ and R₈ are independently H or C₁₋₈ alkyl;
viii) X is O or S; and
ix) Q is C₁₋₃ alkylene optionally substituted with 1 to 4 substituents selected from the group consisting of C₁₋₃ alkyl, C₁₋₄ alkoxy, carboxy, cyano, C₁₋₃ haloalkyl, halogen and oxo; or Q is a bond.

2. The method according to claim 1 wherein R₁ is phenyl or naphthyl each optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, carboxamide, cyano, C₃₋₇ cycloalkyl, halogen, C₁₋₆ haloalkoxy, C₁₋₆ haloalkyl, and hydroxyl.

3. The method according to claim 1 wherein R₁ is phenyl or naphthyl each optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₀, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

4. The method according to claim 1 wherein R₁ is phenyl or naphthyl each optionally substituted with R₉, R₁₀, R₁₁, R₁₂, R₁₃, R₁₄, and R₁₅ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

5. The method according to claim 1 wherein R₁ is phenyl or naphthyl each optionally substituted with R₉, R₁₀, R₁₁, R₁₂, R₁₃, R₁₄, and R₁₅ each selected independently from the group consisting of —OCH₃, —CH₃, cyano, —F, —Cl, —Br, —OCF₃, and —CF₃.

6. The method according to claim 1 wherein R₁ is heteroaryl optionally substituted with R₉, R₁₀, R₁₁, R₁₂, and R₁₃ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

7. The method according to claim 1 wherein R₁ is heteroaryl optionally substituted with R₉, R₁₀, R₁₁, R₁₂, and R₁₃ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

8. The method according to claim 1 wherein R₁ is heteroaryl optionally substituted with R₉, R₁₀, R₁₁, R₁₂, and R₁₃ each selected independently from the group consisting of —OCH₃, —CH₃, cyano, —F, —Cl, —Br, —OCF₃, and —CF₃.

9. The method according to claim 1 wherein R₂ is H or C₁₋₆ alkyl.

10. The method according to claim 1 wherein R₂ is selected from the group consisting of —CH₃, —CH₂CH₃, —CH(CH₃)₂, —CH₂CH₂CH₃, —CH₂CH(CH₃)₂ and —CH₂CH₂CH₂CH₃.

11. The method according to claim 1 wherein R₂ is —CH₃ or —CH(CH₃)₂.

12. The method according to claim 1 wherein R₂ is H.

13. The method according to claim 1 wherein R₃ is H or halogen.

14. The method according to claim 1 wherein R₃ is H, F, Cl, or Br.

15. The method according to claim 1 wherein R₄ is selected from the group consisting of H, C₁₋₆ alkyl and C₁₋₆ haloalkyl.

16. The method according to claim 1 wherein R₄ is H or —CF₃.

17. The method according to claim 1 wherein R₅ is selected from the group consisting of C₁₋₆ alkoxy, C₁₋₆ alkylthio, amino, C₁₋₆ alkylamino, C₂₋₈ dialkylamino, halogen, C₁₋₆ haloalkoxy, and hydroxyl, wherein said C₁₋₆ alkoxy group can be optionally substituted with 1 to 5 substituents selected independently from the group consisting of amino, C₁₋₆ alkylamino, C₂₋₈ dialkylamino, amino, carbo-C₁₋₆-alkoxy, carboxamide, carboxy, cyano, halogen, and phenyl, and wherein said amino and phenyl substituents are each optionally substituted with 1 to 5 further substituents selected from the group consisting of halogen and carbo-C₁₋₆-alkoxy.

18. The method according to claim 1 wherein R₅ is selected from the group consisting of C₁₋₆ alkoxy, C₁₋₆ haloalkoxy, and hydroxyl, wherein said C₁₋₆ alkoxy group can be optionally substituted with 1 to 5 substituents selected independently from the group consisting of amino, C₂₋₈ dialkylamino, carboxy, and phenyl, and wherein said amino and phenyl are each optionally substituted with 1 to 5 further substituents selected from the group consisting of halogen and carbo-C₁₋₆-alkoxy.

19. The method according to claim 1 wherein R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy.

20. The method according to claim 1 wherein R₆ₐ, R_{6b}, and R_{6c} are each independently selected from the group consisting of H, C₁₋₆ alkoxy, C₁₋₆ alkyl, amino, C₁₋₆ alkylamino, C₂₋₈ dialkylamino, cyano, halogen, C₁₋₆ haloalkoxy, C₁₋₆ haloalkyl, hydroxyl, and nitro.

21. The method according to claim 1 wherein R₆ₐ, R_{6b}, and R_{6c} are each independently selected from the group consisting of —H, —OCH₃, —CH₃, —N(CH₃)₂, cyano, —F, —Cl, —Br, —OCF₃, hydroxyl, and nitro.

22. The method according to claim 1 wherein R₆ₐ, R_{6b}, and R_{6c} are all —H.

23. The method according to claim 1 wherein R₇ is —H.

24. The method according to claim 1 wherein R₈ is —H.

25. The method according to claim 1 wherein X is O.

26. The method according to claim 1 wherein X is S.

27. The method according to claim 1 wherein Q is —C(O)—.

28. The method according to claim 1 wherein Q is —CH₂—.

29. The method according to claim 1 wherein Q is a bond.

30. The method according to claim 1, wherein the compound is of Formula (IIa): 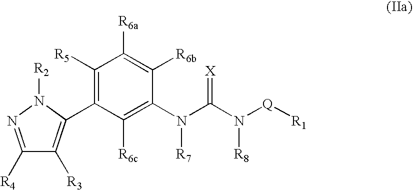
or a pharmaceutically acceptable salt, hydrate or solvate thereof;
wherein:
R₁ is phenyl or naphthyl optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl;
R₂ is C₁₋₆ alkyl;
R₃ is H or halogen;
R₄ is selected from the group consisting of H, C₁₋₆ alkyl and C₁₋₆ haloalkyl;
R₅ is selected from the group consisting of C₁₋₆ alkoxy, C₁₋₆ haloalkoxy, and hydroxyl, wherein said C₁₋₆ alkoxy group can be optionally substituted with 1 to 5 further substituents selected independently from the group consisting of amino, C₂₋₈ dialkylamino, carboxy, and phenyl, and wherein said amino and phenyl are each optionally substituted with 1 to 5 further substituents selected from the group consisting of halogen and carbo-C₁₋₆-alkoxy;
R₆ₐ, R_{6b}, and R_{6c} are each independently selected from the group consisting of H, C₁₋₆ alkoxy, C₁₋₆ alkyl, amino, C₁₋₆ alkylamino, C₂₋₈ dialkylamino, cyano, halogen, C₁₋₆ haloalkoxy, C₁₋₆ haloalkyl, hydroxyl, and nitro;
R₇ and R₈ are both H;
X is O; and
Q is a bond.

31. The method according to claim 1, wherein the compound is of Formula (IIa): 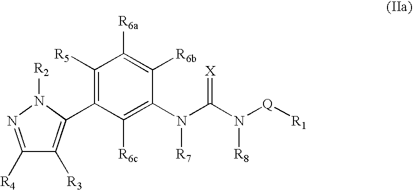
or a pharmaceutically acceptable salt, hydrate or solvate thereof;
wherein:
R₁ is phenyl or naphthyl optionally substituted with R₉, R₁₀, R₁₁, R₁₂, R₁₃, R₁₄, and R₁₅ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl;
R₂ is —CH₃ or —CH(CH₃)₂;
R₃ is —H, —F, —Cl, or —Br;
R₄ is —H, or —CF₃;
R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;
R₆ₐ, R_{6b}, and R_{6c} are each independently selected from the group consisting of —H, —OCH₃, —CH₃, —N(CH₃)₂, cyano, —F, —Cl, —Br, —OCF₃, hydroxyl, and nitro;
R₇ and R₈ are both —H;
X is O; and
Q is a bond.

32. The method according to claim 1, wherein the compound is of Formula (IIa): 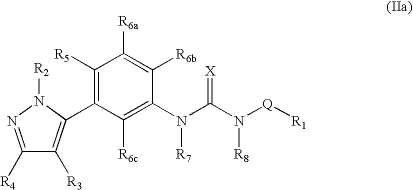
or a pharmaceutically acceptable salt, hydrate or solvate thereof;
wherein:
R₁ is phenyl optionally substituted with R₉, R₁₀, R₁₁, R₁₂, and R₁₃ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl;
R₂ is —CH₃ or —CH(CH₃)₂;
R₃ is —H, —F, —Cl, or —Br;
R₄ is —H, or —CF₃;
R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;
R₆ₐ, R_{6b}, and R_{6c} are each independently selected from the group consisting of —H, —OCH₃, —CH₃, —N(CH₃)₂, cyano, —F, —Cl, —Br, —OCF₃, hydroxyl, and nitro;
R₇ and R₈ are both —H;
X is O; and
Q is a bond.

33. The method according to claim 1, wherein the compound is of Formula (IIa): 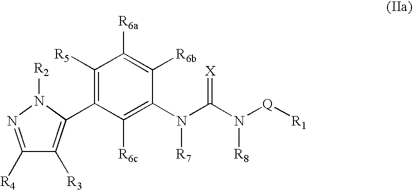
or a pharmaceutically acceptable salt, hydrate or solvate thereof;
wherein:
R₁ is phenyl optionally substituted with R₉, R₁₀, R₁₁, R₁₂, and R₁₃ each selected independently from the group consisting of —C(O)CH₃, —OCH₃, —CH₃, —CH(CH₃)₂, —N(CH₃)₂, cyano, —F, —Cl, —Br, —OCF₃, —CF₃, hydroxyl, and nitro;
R₂ is —CH₃;
R₃ is —H, —F, —Cl, or —Br;
R₄ is —H;
R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;
R₆ₐ, R_{6b}, and R_{6c} are each —H;
R₇ and R₈ are both —H;
X is O; and
Q is a bond.

34. The method according to claim 1 wherein the compound is selected from the group consisting of:
1-[3-(4-Bromo-2-methyl-2H-pyrazol-3-yl)-4-methoxy-phenyl]-3-(4-chloro-phenyl)-urea;
1-[3-(4-Bromo-2-methyl-2H-pyrazol-3-yl)-4-methoxy-phenyl]-3-(4-fluoro-phenyl)-urea;
1-[3-(4-Bromo-2-methyl-2H-pyrazol-3-yl)-4-methoxy-phenyl]-3-(2,4-dichloro-phenyl)-urea;
1-[3-(4-Bromo-2-methyl-2H-pyrazol-3-yl)-4-methoxy-phenyl]-3-(4-methoxy-phenyl)-urea;
1-[3-(4-Bromo-2-methyl-2H-pyrazol-3-yl)-4-methoxy-phenyl]-3-(4-bromo-phenyl)-urea;
1-[3-(4-Bromo-2-methyl-2H-pyrazol-3-yl)-4-methoxy-phenyl]-3-(4-chloro-3-trifluoromethyl-phenyl)-urea;
1-[3-(4-Bromo-2-methyl-2H-pyrazol-3-yl)-4-methoxy-phenyl]-3-(3,5-difluoro-phenyl)-urea;
1-[3-(4-Bromo-2-methyl-2H-pyrazol-3-yl)-4-methoxy-phenyl]-3-(2,4-difluoro-phenyl)-urea;
1-[3-(4-Bromo-2-methyl-2H-pyrazol-3-yl)-4-methoxy-phenyl]-3-(4-chloro-2-trifluoromethyl-phenyl)-urea;
1-[3-(4-Bromo-2-methyl-2H-pyrazol-3-yl)-4-methoxy-phenyl]-3-(3,4-difluoro-phenyl)-urea;
1-[3-(4-Bromo-2-methyl-2H-pyrazol-3-yl)-4-methoxy-phenyl]-3-(3-trifluoromethyl-phenyl)-urea;
1-[3-(4-Bromo-2-methyl-2H-pyrazol-3-yl)-4-methoxy-phenyl]-3-(4-trifluoromethyl-phenyl)-urea;
1-(3,5-Bis-trifluoromethyl-phenyl)-3-[3-(4-bromo-2-methyl-2H-pyrazol-3-yl)-4-methoxy-phenyl]-urea;
1-[3-(4-Bromo-2-methyl-2H-pyrazol-3-yl)-4-methoxy-phenyl]-3-naphthalen-2-yl-urea;
1-[3-(4-Bromo-2-methyl-2H-pyrazol-3-yl)-4-methoxy-phenyl]-3-(3-nitro-phenyl)-urea;
1-[3-(4-Bromo-2-methyl-2H-pyrazol-3-yl)-4-methoxy-phenyl]-3-(4-fluoro-3-nitro-phenyl)-urea;
1-(3-Acetyl-phenyl)-3-[3-(4-bromo-2-methyl-2H-pyrazol-3-yl)-4-methoxy-phenyl]-urea;
1-[3-(4-Bromo-2-methyl-2H-pyrazol-3-yl)-4-methoxy-phenyl]-3-(3-fluoro-phenyl)-urea;
1-[3-(4-Bromo-2-methyl-2H-pyrazol-3-yl)-4-methoxy-phenyl]-3-(4-trifluoromethoxy-phenyl)-urea;
1-[3-(4-Bromo-2-methyl-2H-pyrazol-3-yl)-4-methoxy-phenyl]-3-(3-chloro-phenyl)-urea;
1-[3-(4-Bromo-2-methyl-2H-pyrazol-3-yl)-4-methoxy-phenyl]-3-(3-cyano-phenyl)-urea;
1-Biphenyl-2-yl-3-[3-(4-bromo-2-methyl-2H-pyrazol-3-yl)-4-methoxy-phenyl]-urea;
1-[3-(4-Bromo-2-methyl-2H-pyrazol-3-yl)-4-methoxy-phenyl]-3-(4-isopropyl-phenyl)-urea;
1-[3-(4-Bromo-2-methyl-2H-pyrazol-3-yl)-4-methoxy-phenyl]-3-naphthalen-1-yl-urea;
1-[3-(4-Bromo-2-methyl-2H-pyrazol-3-yl)-4-methoxy-phenyl]-3-(2-fluoro-phenyl)-urea;
1-[3-(4-Chloro-2-methyl-2H-pyrazol-3-yl)-4-methoxy-phenyl]-3-(4-chloro-phenyl)-urea;
1-(4-Chloro-phenyl)-3-[3-(4-fluoro-2-methyl-2H-pyrazol-3-yl)-4-methoxy-phenyl]-urea;
1-[3-(4-Chloro-2-methyl-2H-pyrazol-3-yl)-4-methoxy-phenyl]-3-(4-fluoro-phenyl)-urea;
1-[3-(4-Chloro-2-methyl-2H-pyrazol-3-yl)-4-methoxy-phenyl]-3-(2,4-difluoro-phenyl)-urea;
1-[3-(4-Chloro-2-methyl-2H-pyrazol-3-yl)-4-methoxy-phenyl]-3-(3-methoxy-phenyl)-urea;
1-[3-(4-Fluoro-2-methyl-2H-pyrazol-3-yl)-4-methoxy-phenyl]-3-(4-fluoro-phenyl)-urea;
1-(3,4-Difluoro-phenyl)-3-[3-(4-fluoro-2-methyl-2H-pyrazol-3-yl)-4-methoxy-phenyl]-urea;
1-[3-(4-Fluoro-2-methyl-2H-pyrazol-3-yl)-4-methoxy-phenyl]-3-(3-fluoro-phenyl)-urea;
1-[3-(4-Chloro-2-methyl-2H-pyrazol-3-yl)-4-methoxy-phenyl]-3-(2-trifluoromethoxy-phenyl)-urea;
1-(3-Acetyl-phenyl)-3-[3-(4-chloro-2-methyl-2H-pyrazol-3-yl)-4-methoxy-phenyl]-urea;
1-[3-(4-Chloro-2-methyl-2H-pyrazol-3-yl)-4-methoxy-phenyl]-3-(3-fluoro-phenyl)-urea;
1-(2,4-Difluoro-phenyl)-3-[3-(4-fluoro-2-methyl-2H-pyrazol-3-yl)-4-methoxy-phenyl]-urea;
1-[3-(4-Bromo-2-methyl-5-trifluoromethyl-2H-pyrazol-3-yl)-4-methoxy-phenyl]-3-(4-chloro-phenyl)-urea;
1-[3-(4-Bromo-2-methyl-5-trifluoromethyl-2H-pyrazol-3-yl)-4-methoxy-phenyl]-3-(4-fluoro-phenyl)-urea;
1-[3-(4-Chloro-2-methyl-5-trifluoromethyl-2H-pyrazol-3-yl)-4-methoxy-phenyl]-3-(4-fluoro-phenyl)-urea;
1-[3-(4-Chloro-2-methyl-5-trifluoromethyl-2H-pyrazol-3-yl)-4-methoxy-phenyl]-3-(4-chloro-phenyl)-urea;
1-(4-Chloro-phenyl)-3-[4-methoxy-3-(2-methyl-5-trifluoromethyl-2H-pyrazol-3-yl)-phenyl]-urea;
1-(3-Chloro-phenyl)-3-[3-(2-isopropyl-2H-pyrazol-3-yl)-4-methoxy-phenyl]-urea;
1-(4-Fluoro-phenyl)-3-[3-(2-isopropyl-2H-pyrazol-3-yl)-4-methoxy-phenyl]-urea;
1-[3-(4-Chloro-2-isopropyl-2H-pyrazol-3-yl)-4-methoxy-phenyl]-3-(4-chloro-phenyl)-urea;
1-(3,4-Difluoro-phenyl)-3-[3-(2-isopropyl-2H-pyrazol-3-yl)-4-methoxy-phenyl]-urea;
1-(3-Chloro-4-fluoro-phenyl)-3-[3-(2-isopropyl-2H-pyrazol-3-yl)-4-methoxy-phenyl]-urea;
1-(2-Chloro-4-trifluoromethyl-phenyl)-3-[3-(2-isopropyl-2H-pyrazol-3-yl)-4-methoxy-phenyl]-urea;
1-[3-(4-Bromo-2-isopropyl-2H-pyrazol-3-yl)-4-methoxy-phenyl]-3-(4-chloro-phenyl)-urea;
1-[3-(4-Bromo-2-isopropyl-2H-pyrazol-3-yl)-4-methoxy-phenyl]-3-(4-fluoro-phenyl)-urea;
1-[3-(4-Bromo-2-isopropyl-2H-pyrazol-3-yl)-4-methoxy-phenyl]-3-(3,4-difluoro-phenyl)-urea;
1-[3-(4-Bromo-2-isopropyl-2H-pyrazol-3-yl)-4-methoxy-phenyl]-3-(3-chloro-4-fluoro-phenyl)-urea;
1-[3-(4-Bromo-2-isopropyl-2H-pyrazol-3-yl)-4-methoxy-phenyl]-3-(2-Chloro-4-trifluoromethyl-phenyl)-urea;
1-[3-(4-Chloro-2-isopropyl-2H-pyrazol-3-yl)-4-methoxy-phenyl]-3-(4-fluoro-phenyl)-urea;
1-[3-(4-Chloro-2-isopropyl-2H-pyrazol-3-yl)-4-methoxy-phenyl]-3-(3,4-difluoro-phenyl)-urea;
1-(3-Chloro-4-fluoro-phenyl)-3-[3-(4-Chloro-2-isopropyl-2H-pyrazol-3-yl)-4-methoxy-phenyl]-urea;
1-[3-(4-Chloro-2-isopropyl-2H-pyrazol-3-yl)-4-methoxy-phenyl]-3-(2-Chloro-4-trifluoromethyl-phenyl)-urea;
1-[3-(4-Bromo-2-methyl-2H-pyrazol-3-yl)-4-hydroxy-phenyl]-3-(4-chloro-phenyl)-urea;
1-[3-(4-Bromo-2-methyl-2H-pyrazol-3-yl)-4-isopropoxy-phenyl]-3-(4-chloro-phenyl)-urea;
1-[3-(4-Bromo-2-methyl-2H-pyrazol-3-yl)-4-isopropoxy-phenyl]-3-(4-fluoro-phenyl)-urea;
1-[4-Benzyloxy-3-(4-bromo-2-methyl-2H-pyrazol-3-yl)-phenyl]-3-(4-chloro-phenyl)-urea;
1-[4-Benzyloxy-3-(4-bromo-2-methyl-2H-pyrazol-3-yl)-phenyl]-3-(4-fluoro-phenyl)-urea;
1-[3-(4-Bromo-2-methyl-2H-pyrazol-3-yl)-4-(4-chloro-benzyloxy)-phenyl]-3-(4-chloro-phenyl)-urea;
1-[3-(4-Bromo-2-methyl-2H-pyrazol-3-yl)-4-(4-chloro-benzyloxy)-phenyl]-3-(4-fluoro-phenyl)-urea;
1-[3-(4-Bromo-2-methyl-2H-pyrazol-3-yl)-4-phenethyloxy-phenyl]-3-(4-fluoro-phenyl)-urea;
1-[3-(4-Bromo-2-methyl-2H-pyrazol-3-yl)-4-phenethyloxy-phenyl]-3-(4-chloro-phenyl)-urea;
1-[3-(4-Bromo-2-methyl-2H-pyrazol-3-yl)-4-ethoxy-phenyl]-3-(4-chloro-phenyl)-urea;
1-[3-(4-Bromo-2-methyl-2H-pyrazol-3-yl)-4-ethoxy-phenyl]-3-(4-fluoro-phenyl)-urea;
1-[3-(4-Bromo-2-methyl-2H-pyrazol-3-yl)-4-(2-dimethylamino-ethoxy)-phenyl]-3-(4-chloro-phenyl)-urea;
1-[3-(4-Bromo-2-methyl-2H-pyrazol-3-yl)-4-(2-dimethylamino-ethoxy)-phenyl]-3-(4-fluoro-phenyl)-urea;
1-[3-(4-Bromo-2-methyl-2H-pyrazol-3-yl)-4-methoxy-phenyl]-3-(4-chloro-phenyl)-thiourea;
1-[3-(4-Bromo-2-methyl-2H-pyrazol-3-yl)-4-methoxy-phenyl]-3-(3-methoxy-phenyl)-urea;
1-(4-Chloro-phenyl)-3-[4-methoxy-3-(2-methyl-2H-pyrazol-3-yl)-phenyl]-urea;
1-[3-(4-Chloro-2-methyl-2H-pyrazol-3-yl)-4-methoxy-phenyl]-3-(4-isopropyl-phenyl)-urea;
1-[3-(4-Chloro-2-methyl-2H-pyrazol-3-yl)-4-methoxy-phenyl]-3-(2,4-dichloro-phenyl)-urea;
1-[3-(4-Chloro-2-methyl-2H-pyrazol-3-yl)-4-methoxy-phenyl]-3-naphthalen-1-yl-urea;
1-[3-(4-Chloro-2-methyl-2H-pyrazol-3-yl)-4-methoxy-phenyl]-3-(4-chloro-2-trifluoromethyl-phenyl)-urea;
1-[3-(4-Chloro-2-methyl-2H-pyrazol-3-yl)-4-methoxy-phenyl]-3-(4-trifluoromethyl-phenyl)-urea;
1-(4-Bromo-phenyl)-3-[3-(4-chloro-2-methyl-2H-pyrazol-3-yl)-4-methoxy-phenyl]-urea;
1-(3,5-Bis-trifluoromethyl-phenyl)-3-[3-(4-chloro-2-methyl-2H-pyrazol-3-yl)-4-methoxy-phenyl]-urea;
1-(3-Chloro-phenyl)-3-[3-(4-fluoro-2-methyl-2H-pyrazol-3-yl)-4-methoxy-phenyl]-urea;
1-(4-Chloro-3-trifluoromethyl-phenyl)-3-[3-(4-fluoro-2-methyl-2H-pyrazol-3-yl)-4-methoxy-phenyl]-urea;
1-(4-Bromo-phenyl)-3-[3-(4-fluoro-2-methyl-2H-pyrazol-3-yl)-4-methoxy-phenyl]-urea;
1-[3-(4-Fluoro-2-methyl-2H-pyrazol-3-yl)-4-methoxy-phenyl]-3-(4-trifluoromethyl-phenyl)-thiourea;
1-[3-(4-Fluoro-2-methyl-2H-pyrazol-3-yl)-4-methoxy-phenyl]-3-(4-methoxy-phenyl)-urea;
1-(3-Acetyl-phenyl)-3-[3-(4-fluoro-2-methyl-2H-pyrazol-3-yl)-4-methoxy-phenyl]-urea;
1-[3-(4-Fluoro-2-methyl-2H-pyrazol-3-yl)-4-methoxy-phenyl]-3-(4-trifluoromethyl-phenyl)-urea; and
1-[3-(4-Fluoro-2-methyl-2H-pyrazol-3-yl)-4-methoxy-phenyl]-3-(3-trifluoromethyl-phenyl)-urea, or a pharmaceutically acceptable salt, hydrate or solvate thereof.

35. The method according to claim 1 wherein the compound is selected from the group consisting of:
1-[3-(4-Chloro-2-methyl-2H-pyrazol-3-yl)-4-methoxy-phenyl]-3-(3-chloro-phenyl)-urea;
1-[3-(4-Chloro-2-methyl-2H-pyrazol-3-yl)-4-methoxy-phenyl]-3-(3,4-difluoro-phenyl)-urea;
1-[3-(4-Chloro-2-methyl-2H-pyrazol-3-yl)-4-methoxy-phenyl]-3-(3,5-difluoro-phenyl)-urea;
1-[3-(4-Bromo-2-methyl-2H-pyrazol-3-yl)-4-methoxy-phenyl]-3-[3-(1-hydroxy-ethyl)-phenyl]-urea;
1-[3-(4-Bromo-2-methyl-2H-pyrazol-3-yl)-4-methoxy-phenyl]-3-[3-(1-hydroxyimino-ethyl)-phenyl]-urea;
1-[3-(4-Chloro-2-methyl-2H-pyrazol-3-yl)-4-methoxy-phenyl]-3-(2-fluoro-phenyl)-urea;
1-(4-Chloro-phenyl)-3-[3-(2-methyl-2H-pyrazol-3-yl)-4-trifluoromethoxy-phenyl]-urea;
1-(2,4-Difluoro-phenyl)-3-[3-(2-methyl-2H-pyrazol-3-yl)-4-trifluoromethoxy-phenyl]-urea;
1-(4-Fluoro-phenyl)-3-[3-(2-methyl-2H-pyrazol-3-yl)-4-trifluoromethoxy-phenyl]-urea;
1-[3-(2-Methyl-2H-pyrazol-3-yl)-4-trifluoromethoxy-phenyl]-3-(4-trifluoromethyl-phenyl)-urea;
1-[3-(4-Bromo-2-methyl-2H-pyrazol-3-yl)-4-methoxy-phenyl]-3-[4-chloro-2-(4-methyl-piperazin-1-yl)-phenyl]-urea;
1-[3-(4-Bromo-2-methyl-2H-pyrazol-3-yl)-4-hydroxy-phenyl]-3-(2,4-difluoro-phenyl)-urea;
1-[3-(4-Bromo-2-methyl-2H-pyrazol-3-yl)-4-methoxy-phenyl]-3-(4-chloro-2-morpholin-4-yl-phenyl)-urea;
1-[3-(4-Bromo-2-methyl-2H-pyrazol-3-yl)-4-methoxy-phenyl]-3-[4-chloro-2-(4-methyl-piperidin-1-yl)-phenyl]-urea;
1-[3-(4-Bromo-2-methyl-2H-pyrazol-3-yl)-4-methoxy-phenyl]-3-(4-chloro-2-hydroxy-phenyl)-urea;
1-[3-(4-Bromo-2-methyl-2H-pyrazol-3-yl)-4-trifluoromethoxy-phenyl]-3-(4-chloro-phenyl)-urea;
1-[3-(4-Chloro-2-methyl-2H-pyrazol-3-yl)-4-methoxy-phenyl]-3-(3-cyano-phenyl)-urea;
1-[3-(4-Chloro-2-methyl-2H-pyrazol-3-yl)-4-methoxy-phenyl]-3-(3-nitro-phenyl)-urea;
1-[3-(4-Bromo-2-methyl-2H-pyrazol-3-yl)-4-methoxy-phenyl]-3-{4-chloro-2-[(2-dimethylamino-ethyl)-methyl-amino]-phenyl}-urea;
1-[3-(4-Bromo-2-methyl-2H-pyrazol-3-yl)-4-methoxy-phenyl]-3-{4-chloro-2-[(3-dimethylamino-propyl)-methyl-amino]-phenyl}-urea;
1-[3-(4-Bromo-2-methyl-2H-pyrazol-3-yl)-4-trifluoromethoxy-phenyl]-3-(2,4-difluoro-phenyl)-urea;
1-(3-Acetyl-phenyl)-3-[3-(2-methyl-2H-pyrazol-3-yl)-4-trifluoromethoxy-phenyl]-urea;
1-[3-(4-Bromo-2-methyl-2H-pyrazol-3-yl)-4-methoxy-phenyl]-3-(2,2-difluoro-benzo[1,3]dioxol-5-yl)-urea;
1-[3-(4-Bromo-2-methyl-2H-pyrazol-3-yl)-4-methoxy-phenyl]-3-(4-dimethylamino-phenyl)-urea;
1-[3-(4-Bromo-2-methyl-2H-pyrazol-3-yl)-4-(3-dimethylamino-propoxy)-phenyl]-3-(4-chloro-phenyl)-urea;
{2-(4-Bromo-2-methyl-2H-pyrazol-3-yl)-4-[3-(4-chloro-phenyl)-ureido]-phenoxy}-acetic acid;
1-(4-Chloro-phenyl)-3-[4-hydroxy-3-(2-methyl-2H-pyrazol-3-yl)-phenyl]-urea;
1-[3-(4-Chloro-2-methyl-2H-pyrazol-3-yl)-4-hydroxy-phenyl]-3-(2,4-difluoro-phenyl)-urea;
1-[3-(4-Chloro-2-methyl-2H-pyrazol-3-yl)-4-hydroxy-phenyl]-3-(4-chloro-phenyl)-urea;
1-(4-Chloro-phenyl)-3-[4-(3-dimethylamino-propoxy)-3-(2-methyl-2H-pyrazol-3-yl)-phenyl]-urea;
1-[3-(4-Bromo-2-methyl-2H-pyrazol-3-yl)-4-(2-dimethylamino-ethoxy)-phenyl]-3-(2,4-difluoro-phenyl)-urea;
1-(2,4-Difluoro-phenyl)-3-[4-(3-dimethylamino-propoxy)-3-(2-methyl-2H-pyrazol-3-yl)-phenyl]-urea;
1-[4-(3-Dimethylamino-propoxy)-3-(2-methyl-2H-pyrazol-3-yl)-phenyl]-3-(4-fluoro-phenyl)-urea;
1-(4-Chloro-phenyl)-3-[4-(2-dimethylamino-ethoxy)-3-(2-methyl-2H-pyrazol-3-yl)-phenyl]-urea;
1-[3-(4-Chloro-2-methyl-2H-pyrazol-3-yl)-4-(3-dimethylamino-propoxy)-phenyl]-3-(4-chloro-phenyl)-urea;
1-(2,2-Difluoro-benzo[1,3]dioxol-5-yl)-3-[4-(3-dimethylamino-propoxy)-3-(2-methyl-2H-pyrazol-3-yl)-phenyl]-urea;
1-[4-(3-Dimethylamino-propoxy)-3-(2-methyl-2H-pyrazol-3-yl)-phenyl]-3-p-tolyl-urea;
1-[4-(3-Dimethylamino-propoxy)-3-(2-methyl-2H-pyrazol-3-yl)-phenyl]-3-(4-methoxy-phenyl)-urea;
1-[3-(4-Chloro-2-methyl-2H-pyrazol-3-yl)-4-(2-dimethylamino-ethoxy)-phenyl]-3-(2,4-difluoro-phenyl)-urea;
1-[3-(4-Chloro-2-methyl-2H-pyrazol-3-yl)-4-(3-dimethylamino-propoxy)-phenyl]-3-(2,4-difluoro-phenyl)-urea;
1-(3-Chloro-phenyl)-3-[4-(3-dimethylamino-propoxy)-3-(2-methyl-2H-pyrazol-3-yl)-phenyl]-urea;
1-(3-Chloro-4-fluoro-phenyl)-3-[4-(3-dimethylamino-propoxy)-3-(2-methyl-2H-pyrazol-3-yl)-phenyl]-urea;
1-(3,4-Difluoro-phenyl)-3-[4-(3-dimethylamino-propoxy)-3-(2-methyl-2H-pyrazol-3-yl)-phenyl]-urea;
1-[4-(3-Dimethylamino-propoxy)-3-(2-methyl-2H-pyrazol-3-yl)-phenyl]-3-(4-trifluoromethyl-phenyl)-urea;
1-[4-(3-Dimethylamino-propoxy)-3-(2-methyl-2H-pyrazol-3-yl)-phenyl]-3-(2-fluoro-phenyl)-urea;
1-[4-(3-Dimethylamino-propoxy)-3-(2-methyl-2H-pyrazol-3-yl)-phenyl]-3-(2-fluoro-5-methyl-phenyl)-urea;
1-(2-Chloro-phenyl)-3-[4-(3-dimethylamino-propoxy)-3-(2-methyl-2H-pyrazol-3-yl)-phenyl]-urea;
1-(2,4-Difluoro-phenyl)-3-[4-(2-dimethylamino-ethoxy)-3-(2-methyl-2H-pyrazol-3-yl)-phenyl]-urea;
1-[4-(2-Dimethylamino-ethoxy)-3-(2-methyl-2H-pyrazol-3-yl)-phenyl]-3-(4-fluoro-phenyl)-urea;
1-(3-Acetyl-phenyl)-3-[4-(2-dimethylamino-ethoxy)-3-(2-methyl-2H-pyrazol-3-yl)-phenyl]-urea;
1-(2,2-Difluoro-benzo[1,3]dioxol-5-yl)-3-[4-(2-dimethylamino-ethoxy)-3-(2-methyl-2H-pyrazol-3-yl)-phenyl]-urea;
1-[4-(3-Dimethylamino-propoxy)-3-(2-methyl-2H-pyrazol-3-yl)-phenyl]-3-phenyl-urea;
1-[4-(2-Dimethylamino-ethoxy)-3-(2-methyl-2H-pyrazol-3-yl)-phenyl]-3-(3-methoxy-phenyl)-urea;
(2-{2-(4-Bromo-2-methyl-2H-pyrazol-3-yl)-4-[3-(2,4-difluoro-phenyl)-ureido]-phenoxy}-ethyl)-carbamic acid tert-butyl ester;
1-[3-(4-Bromo-2-methyl-2H-pyrazol-3-yl)-4-(3-dimethylamino-propoxy)-phenyl]-3-(2,4-difluoro-phenyl)-urea;
1-[3-(4-Bromo-2-methyl-2H-pyrazol-3-yl)-4-(3-dimethylamino-propoxy)-phenyl]-3-(2-chloro-phenyl)-urea;
1-[3-(4-Bromo-2-methyl-2H-pyrazol-3-yl)-4-(3-dimethylamino-propoxy)-phenyl]-3-(2-fluoro-phenyl)-urea;
1-(4-Chloro-phenyl)-3-[4-methoxy-3-(2H-pyrazol-3-yl)-phenyl]-urea;
1-[3-(4-Bromo-2H-pyrazol-3-yl)-4-methoxy-phenyl]-3-(2,4-difluoro-phenyl)-urea;
1-(2,4-Difluoro-phenyl)-3-[4-methoxy-3-(2H-pyrazol-3-yl)-phenyl]-urea; and
1-(4-Chloro-phenyl)-3-[4-hydroxy-3-(1-methyl-1H-pyrazol-3-yl)-phenyl]-urea, or a pharmaceutically acceptable salt, hydrate or solvate thereof.

36. The method according to claim 1 wherein the compound is selected from the group consisting of:
1-Benzoyl-3-[3-(4-bromo-2-methyl-2H-pyrazol-3-yl)-4-methoxy-phenyl]-urea; and
1-Benzyl-3-[3-(4-bromo-2-methyl-2H-pyrazol-3-yl)-4-methoxy-phenyl]-urea, or a pharmaceutically acceptable salt, hydrate or solvate thereof.

37. The method according to claim 1 wherein the compound is selected from the group consisting of:
1-Benzoyl-3-[3-(4-chloro-2-methyl-2H-pyrazol-3-yl)-4-methoxy-phenyl]-urea;
1-Benzyl-3-[3-(4-chloro-2-methyl-2H-pyrazol-3-yl)-4-methoxy-phenyl]-urea; and
1-(4-Chloro-benzyl)-3-[4-(3-dimethylamino-propoxy)-3-(2-methyl-2H-pyrazol-3-yl)-phenyl]-urea, or a pharmaceutically acceptable salt, hydrate or solvate thereof.

38. The method according to claim 1 wherein the compound is selected from the group consisting of:
1-(4-Chloro-phenyl)-3-[4-(2-dimethylamino-ethoxy)-3-(4-fluoro-2-methyl-2H-pyrazol-3-yl)-phenyl]-urea;
1-[4-(2-Dimethylamino-ethoxy)-3-(4-fluoro-2-methyl-2H-pyrazol-3-yl)-phenyl]-3-(4-fluoro-phenyl)-urea;
1-(2,4-Difluoro-phenyl)-3-[4-(2-dimethylamino-ethoxy)-3-(4-fluoro-2-methyl-2H-pyrazol-3-yl)-phenyl]-urea;
1-(4-Chloro-2-hydroxy-phenyl)-3-[4-(2-dimethylamino-ethoxy)-3-(4-fluoro-2-methyl-2H-pyrazol-3-yl)-phenyl]-urea;
1-[4-(2-Dimethylamino-ethoxy)-3-(4-fluoro-2-methyl-2H-pyrazol-3-yl)-phenyl]-3-(4-fluoro-2-hydroxy-phenyl)-urea;
1-(4-Chloro-3-hydroxy-phenyl)-3-[4-(2-dimethylamino-ethoxy)-3-(4-fluoro-2-methyl-2H-pyrazol-3-yl)-phenyl]-urea;
1-[4-(2-Dimethylamino-ethoxy)-3-(4-fluoro-2-methyl-2H-pyrazol-3-yl)-phenyl]-3-(4-fluoro-3-hydroxy-phenyl)-urea;
1-[3-(4-Chloro-2-methyl-2H-pyrazol-3-yl)-4-(2-dimethylamino-ethoxy)-phenyl]-3-(4-chloro-phenyl)-urea;
1-[3-(4-Chloro-2-methyl-2H-pyrazol-3-yl)-4-(2-dimethylamino-ethoxy)-phenyl]-3-(4-fluoro-phenyl)-urea;
1-(4-Chloro-2-hydroxy-phenyl)-3-[3-(4-chloro-2-methyl-2H-pyrazol-3-yl)-4-(2-dimethylamino-ethoxy)-phenyl]-urea;
1-[3-(4-Chloro-2-methyl-2H-pyrazol-3-yl)-4-(2-dimethylamino-ethoxy)-phenyl]-3-(4-fluoro-2-hydroxy-phenyl)-urea;
1-(4-Chloro-3-hydroxy-phenyl)-3-[3-(4-chloro-2-methyl-2H-pyrazol-3-yl)-4-(2-dimethylamino-ethoxy)-phenyl]-urea;
1-[3-(4-Chloro-2-methyl-2H-pyrazol-3-yl)-4-(2-dimethylamino-ethoxy)-phenyl]-3-(4-fluoro-3-hydroxy-phenyl)-urea;
1-(4-Chloro-2-hydroxy-phenyl)-3-[4-(2-dimethylamino-ethoxy)-3-(2-methyl-2H-pyrazol-3-yl)-phenyl]-urea;
1-[4-(2-Dimethylamino-ethoxy)-3-(2-methyl-2H-pyrazol-3-yl)-phenyl]-3-(4-fluoro-2-hydroxy-phenyl)-urea;
1-(4-Chloro-3-hydroxy-phenyl)-3-[4-(2-dimethylamino-ethoxy)-3-(2-methyl-2H-pyrazol-3-yl)-phenyl]-urea;
1-[4-(2-Dimethylamino-ethoxy)-3-(2-methyl-2H-pyrazol-3-yl)-phenyl]-3-(4-fluoro-3-hydroxy-phenyl)-urea;
1-[3-(4-Bromo-2-methyl-2H-pyrazol-3-yl)-4-(2-dimethylamino-ethoxy)-phenyl]-3-(4-chloro-2-hydroxy-phenyl)-urea;
1-[3-(4-Bromo-2-methyl-2H-pyrazol-3-yl)-4-(2-dimethylamino-ethoxy)-phenyl]-3-(4-fluoro-2-hydroxy-phenyl)-urea;
1-[3-(4-Bromo-2-methyl-2H-pyrazol-3-yl)-4-(2-dimethylamino-ethoxy)-phenyl]-3-(4-chloro-3-hydroxy-phenyl)-urea;
1-[3-(4-Bromo-2-methyl-2H-pyrazol-3-yl)-4-(2-dimethylamino-ethoxy)-phenyl]-3-(4-fluoro-3-hydroxy-phenyl)-urea;
1-(4-Chloro-phenyl)-3-[4-(3-dimethylamino-propoxy)-3-(4-fluoro-2-methyl-2H-pyrazol-3-yl)-phenyl]-urea;
1-[4-(3-Dimethylamino-propoxy)-3-(4-fluoro-2-methyl-2H-pyrazol-3-yl)-phenyl]-3-(4-fluoro-phenyl)-urea;
1-(2,4-Difluoro-phenyl)-3-[4-(3-dimethylamino-propoxy)-3-(4-fluoro-2-methyl-2H-pyrazol-3-yl)-phenyl]-urea;
1-(4-Chloro-2-hydroxy-phenyl)-3-[4-(3-dimethylamino-propoxy)-3-(4-fluoro-2-methyl-2H-pyrazol-3-yl)-phenyl]-urea;
1-[4-(3-Dimethylamino-propoxy)-3-(4-fluoro-2-methyl-2H-pyrazol-3-yl)-phenyl]-3-(4-fluoro-2-hydroxy-phenyl)-urea;
1-(4-Chloro-3-hydroxy-phenyl)-3-[4-(3-dimethylamino-propoxy)-3-(4-fluoro-2-methyl-2H-pyrazol-3-yl)-phenyl]-urea;
1-[4-(3-Dimethylamino-propoxy)-3-(4-fluoro-2-methyl-2H-pyrazol-3-yl)-phenyl]-3-(4-fluoro-3-hydroxy-phenyl)-urea;
1-[3-(4-Chloro-2-methyl-2H-pyrazol-3-yl)-4-(3-dimethylamino-propoxy)-phenyl]-3-(4-fluoro-phenyl)-urea;
1-(4-Chloro-2-hydroxy-phenyl)-3-[3-(4-chloro-2-methyl-2H-pyrazol-3-yl)-4-(3-dimethylamino-propoxy)-phenyl]-urea;
1-[3-(4-Chloro-2-methyl-2H-pyrazol-3-yl)-4-(3-dimethylamino-propoxy)-phenyl]-3-(4-fluoro-2-hydroxy-phenyl)-urea;
1-(4-Chloro-3-hydroxy-phenyl)-3-[3-(4-chloro-2-methyl-2H-pyrazol-3-yl)-4-(3-dimethylamino-propoxy)-phenyl]-urea;
1-[3-(4-Chloro-2-methyl-2H-pyrazol-3-yl)-4-(3-dimethylamino-propoxy)-phenyl]-3-(4-fluoro-3-hydroxy-phenyl)-urea;
1-(4-Chloro-2-hydroxy-phenyl)-3-[4-(3-dimethylamino-propoxy)-3-(2-methyl-2H-pyrazol-3-yl)-phenyl]-urea;
1-[4-(3-Dimethylamino-propoxy)-3-(2-methyl-2H-pyrazol-3-yl)-phenyl]-3-(4-fluoro-2-hydroxy-phenyl)-urea;
1-(4-Chloro-3-hydroxy-phenyl)-3-[4-(3-dimethylamino-propoxy)-3-(2-methyl-2H-pyrazol-3-yl)-phenyl]-urea;
1-[4-(3-Dimethylamino-propoxy)-3-(2-methyl-2H-pyrazol-3-yl)-phenyl]-3-(4-fluoro-3-hydroxy-phenyl)-urea;
1-[3-(4-Bromo-2-methyl-2H-pyrazol-3-yl)-4-(3-dimethylamino-propoxy)-phenyl]-3-(4-fluoro-phenyl)-urea;
1-[3-(4-Bromo-2-methyl-2H-pyrazol-3-yl)-4-(3-dimethylamino-propoxy)-phenyl]-3-(4-chloro-2-hydroxy-phenyl)-urea;
1-[3-(4-Bromo-2-methyl-2H-pyrazol-3-yl)-4-(3-dimethylamino-propoxy)-phenyl]-3-(4-fluoro-2-hydroxy-phenyl)-urea;
1-[3-(4-Bromo-2-methyl-2H-pyrazol-3-yl)-4-(3-dimethylamino-propoxy)-phenyl]-3-(4-chloro-3-hydroxy-phenyl)-urea; and
1-[3-(4-Bromo-2-methyl-2H-pyrazol-3-yl)-4-(3-dimethylamino-propoxy)-phenyl]-3-(4-fluoro-3-hydroxy-phenyl)-urea, or a pharmaceutically acceptable salt, hydrate or solvate thereof.

39. The method according to claim 1, wherein the compound is a 5-HT_{2A} inverse agonist.

40. The method according to claim 1, wherein the individual in need thereof is a human.

41. The method according to claim 1, wherein the individual in need thereof has a lymphoproliferative disorder.

42. The method according to claim 1, wherein the individual in need thereof is immunocompromised.

43. The method according to claim 1, wherein the individual in need thereof is infected with HIV.

44. The method according to claim 1, wherein the individual in need thereof is infected with HIV, and the HIV-infected individual has a CD4+ cell count of ≦200/mm³.

45. The method according to claim 1, wherein the individual in need thereof is infected with HIV, and wherein the individual has AIDS.

46. The method according to claim 1, wherein the individual in need thereof is infected with HIV, and wherein the individual has AIDS-related complex (ARC).

47. The method according to claim 1, wherein the individual in need thereof is undergoing immunosuppressive therapy.

48-59. (canceled)
